(19) United States
(12) Patent Application Publication
(10) Pub. No.: US 2026/0077056 A1
(43) Pub. Date: Mar. 19, 2026

(54) LINKER-PAYLOAD COMPOUND, CONJUGATES AND APPLICATIONS THEREOF

(71) Applicants: GENEQUANTUM HEALTHCARE (SUZHOU) CO., LTD., Suzhou (CN); GENEQUANTUM MEDICINE (SUZHOU) CO., LTD., Suzhou (CN)

(72) Inventors: Gang QIN, Suzhou (CN); Zhaoxiong CAI, Suzhou (CN); Mingyu HU, Suzhou (CN)

(21) Appl. No.: 19/121,119

(22) PCT Filed: Oct. 13, 2023
(86) PCT No.: PCT/CN2023/124467

§ 371 Date: Apr. 14, 2025

(30) Foreign Application Priority Data
Oct. 14, 2022 (WO) PCT/CN2022/125371

(51) Int. Cl. A61K 47/68; A61P 35/00; C07K 16/28; C07K 16/32

(52) U.S. Cl. CPC A61K 47/6883; A61K 47/6803; A61K 47/6849; A61K 47/6855; A61K 47/6889; A61P 35/00; C07K 16/2863; C07K 16/32

(57) ABSTRACT

A linker-payload compound for targeting molecule-drug conjugate, and the corresponding conjugate, the preparation and use thereof are provided.

### TECHNICAL FIELD

The present disclosure relates to the biopharmaceutical field, in particular, to a linker-payload compound for preparing targeting molecule-drug conjugates, and the corresponding conjugates, the preparing process and use thereof.

### BACKGROUND

Eribulin is a small molecule with the structure shown below. It acts by interfering with the microtubular growth ultimately leading to apoptosis after prolonged mitotic blockage. Non-clinical studies have demonstrated the unique role of Eribulin in the tumor microenvironment, such as increasing the vascular perfusion and permeability of the core area of the tumor, improving the status of epithelial cells, and reducing the migration capacity of breast cancer cells. At present, Eribulin has been approved in more than 70 countries and regions in Europe, America and Asia, and was approved by National Medical Products Administration in China in 2019. In patients with metastatic breast cancer refractory to anthracyclines and taxanes, eribulin is one of the life-saving options. Eribulin has also been shown to cause less peripheral neuropathy compared with other MTAs in animal models and clinically.

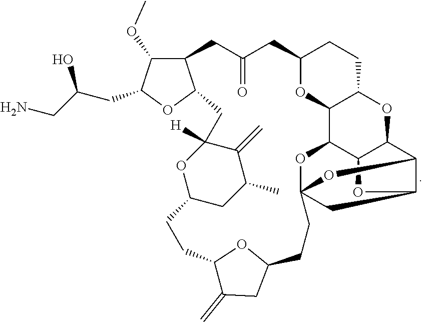 [SMILES: C=C1C[C@@H]2CC[C@@]34C[C@H]5O[C@H]6[C@@H](O3)[C@H]3O[C@H](CC[C@@H]3O[C@H]6[C@H]5O4)CC(=O)C[C@@H]3[C@@H](OC)[C@@H](C[C@H](O)CN)O[C@H]3C[C@H]3O[C@@H](CC[C@@H]1O2)C[C@@H](C)C3=C]

With well verified safety and outstanding pharmaceutical properties, Eribulin hold a promise to serve as a payload and to form an ADC which may achieve a better balanced safety-efficacy profile.

The ADC MORAb-202 in research that applies eribulin as the payload demonstrates potent antitumor activity and may become a potential new treatment modality for FRA-positive cancers.

The ADC Enhertu targeting HER2, the ADC LY3076226 targeting FGFR3, and the ADC MORAb-202, as well as the other commercially available ADCs and most of the ADCs in clinical trials, are prepared by chemical conjugation. Enhertu and MORAb-202 are both prepared using a thiosuccinimide structure (thiosuccinimide linkage) to conjugate the small molecule drug with the targeting antibody or protein. The thiosuccinimide structure is formed by the Michael addition between thiol group and maleimide group. However, the thiosuccinimide linkage is unstable due to potential reverse Michael addition or thio-exchanging under physiological condition, which may lead to the premature release of the cytotoxin in the circulation and enhanced off-target toxicity, and eventually reduces the safety and limits the clinical application of the ADC drug candidate.

The development of a safe and effective ADC drug candidate involves a profound understanding of the four major components, i.e. antigen selection, payload potency, linker design and conjugation method. The selection of linker connecting the cytotoxic payload and mAb is one of the major challenges in the discovery of a potent ADC, as linker is crucial to the PK properties, selectivity, therapeutic index of the ADC. We have a long interest in the development of stable linker, from which bears the fruits including a prolonged half life of ADC drugs in systemic circulation, decreased premature fall-off and increased accumulation of the payload in the target (See our stable linker technology: CN106856656B). Here we describe a combination of less investigated ADC payload (i.e. Eribulin) and our unique stable linker as a novel platform for the discovery of more potent, stable and safer ADC drugs. This technology will be beneficial for addressing the unmet clinical need of cancer patients worldwide.

### SUMMARY

In a first aspect, provided is a compound having the structure of formula (II):

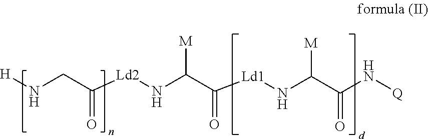

wherein,
Q is hydrogen or LKb-P;
M is hydrogen or Lka-LKb-P;
provided that Q and M are not simultaneously hydrogen;
each Lka is independently selected from

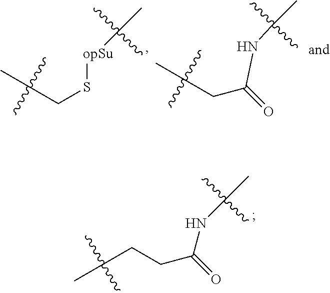

opSu is

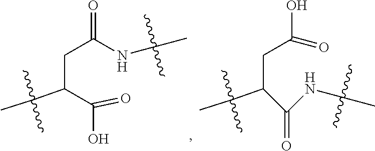 [SMILES: *NC(=O)CC(*)C(=O)O ; *NC(=O)C(*)CC(=O)O]

or a mixture thereof;
each LKb is independently L²-L¹;
P is a payload which is linked to the L¹ moiety;
L′ is Cleavable sequence 1 comprising an amino acid sequence which can be cleaved by enzyme, and Cleavable sequence 1 comprises 1-10 amino acids;
L² is a bond; or a C₂₋₂₀ alkylene wherein one or more —CH₂— structures in the alkylene is optionally replaced by —CR¹R²—, —O—, —(CO)—, —S(═O)₂—, —NR₃—, —N+R⁴R⁵—, C₄₋₁₀ cycloalkylene, C₄₋₁₀ heterocyclylene, phenylene; wherein the cycloalkylene, heterocyclylene and phenylene are each independently unsubstituted or substituted with at least one substituent selected from halogen, —C₁₋₁₀ alkyl, —C₁₋₁₀ haloalkyl, —C₁₋₁₀ alkylene-NH—R⁶ and —C₁₋₁₀ alkylene-O—R⁷;
Ld2 and each Ld1 are independently a bond; or selected from —NH—C₁₋₂₀ alkylene-(CO)—, —NH-(PEG); —(CO)—, or is a natural amino acid or oligomeric natural amino acids having a degree of polymerization of 2-10 independently unsubstituted or substituted with -(PEG)ⱼ-R⁸ on the side chain;
-(PEG)ᵢ- and -(PEG)ⱼ- are each a PEG fragment, which comprises the denoted number of consecutive —(O—C₂H₄)- structure units or consecutive —(C₂H₄—O)— structure units, with an optional additional C₁₋₁₀ alkylene at one terminal;
R¹, R², R³, R⁴, R⁵, R⁶, R⁷ are each independently selected from hydrogen, halogen, —C₁₋₁₀ alkyl, —C₁₋₁₀ haloalkyl, C₄₋₁₀ cycloalkylene;
R⁸ is C₁-10 alkyl;
n is any integer of 2 to 20;
d is 0, or is any integer of 1 to 6;
each i is independently an integer of 1-100, preferably 1 to 20; preferably each i is independently an integer of 1 to 12; more preferably 2 to 8; particularly 4;
each j is independently an integer of 1-100, preferably 1 to 20; preferably each j is independently an integer of 1 to 12; more preferably 8 to 12; particularly 8 or 12.

In one embodiment, in the compound of formula (II), P has the structure of formula (i)

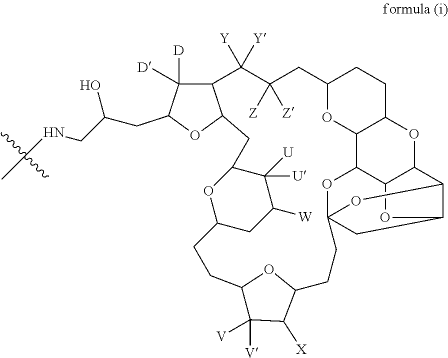

wherein,
each of D and D′ is independently selected from R⁹ and OR⁹, wherein R⁹ is H, C₁₋₃ alkyl, or C₁₋₃ haloalkyl;
each of U and U′ is independently H, C₁₋₃ alkoxy, or C₁₋₃ alkyl; or U and U′ taken together are ═CH₂;
W is C₁₋₃ alkyl;
each of V and V′ is independently H, C₁₋₃ alkoxy, or C₁₋₃ alkyl; or U and U′ taken together are ═CH₂;
X is H or C₁₋₆ alkoxy;
each of Y and Y′ is independently H or C₁₋₃ alkoxy; or Y and Y′ taken together are ═O, ═CH₂; and
each of Z and Z′ is independently H or C₁₋₃ alkoxy; or Z and Z′ taken together are ═O, ═CH₂;
or a pharmaceutically acceptable salt thereof;
preferably, P has the structure of formula (i-1)

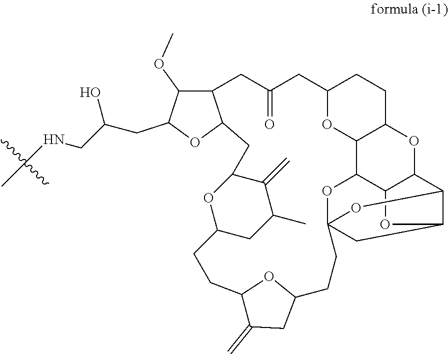 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]

further preferably, P has the structure of formula (ii)

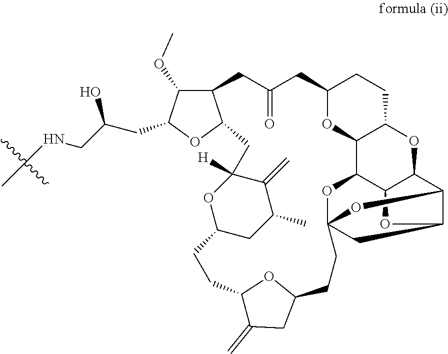 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

wherein the wavy bond shows the connection site for connection to the L′ moiety.

In a second aspect, provided is a conjugate having the structure of formula (III):

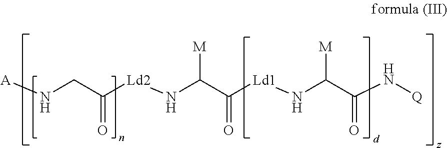

wherein,
Q is hydrogen or LKb-P;
M is hydrogen or LKa-LKb-P;
provided that Q and M are not simultaneously hydrogen;
A is a targeting molecule which is linked to the Gₙ moiety of the compound of formula (II); G is glycine;
z is an integer of 1 to 20;
P, n, d, Ld1, Ld2, LKa and LKb are as defined in formula (II).

In one embodiment, in the conjugate of formula (III), P has the structure of formula (i)

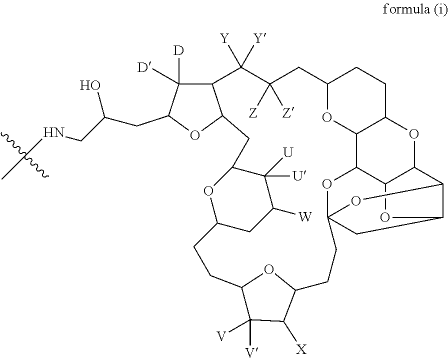

wherein,
each of D and D′ is independently selected from R⁹ and OR⁹, wherein R⁹ is H, C₁₋₃ alkyl, or C₁₋₃ haloalkyl;
each of U and U′ is independently H, C₁₋₃ alkoxy, or C₁₋₃ alkyl; or U and U′ taken together are ═CH₂;
W is C₁₋₃ alkyl;
each of V and V′ is independently H, C₁₋₃ alkoxy, or C₁₋₃ alkyl; or U and U′ taken together are ═CH₂;
X is H or C₁₋₆ alkoxy;
each of Y and Y′ is independently H or C₁₋₃ alkoxy; or Y and Y′ taken together are ═O, ═CH₂; and
each of Z and Z′ is independently H or C₁₋₃ alkoxy; or Z and Z′ taken together are ═O, ═CH₂;
or a pharmaceutically acceptable salt thereof;
preferably, P has the structure of formula (i-1)

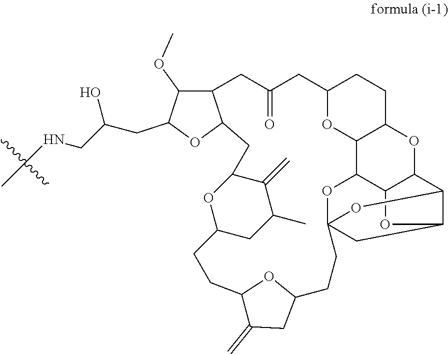 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]

further preferably, P has the structure of formula (ii)

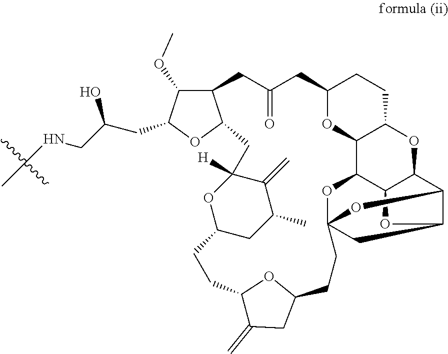 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

wherein the wavy bond shows the connection site for connection to the L′ moiety;
and or
(ii) the targeting molecule is antibody or antigen binding fragment thereof; preferably, antibody is anti-CD19 antibody, anti-CD20 antibody, anti-CD22 antibody, anti-CD25 antibody, anti-CD30/TNFRSF8 antibody, anti-CD33 antibody, anti-CD37 antibody, anti-CD44v6 antibody, anti-CD56 antibody, anti-CD70 antibody, anti-CD71 antibody, anti-CD74 antibody, anti-CD79b antibody, anti-CD117/KITk antibody, anti-CD123 antibody, anti-CD138 antibody, anti-CD142 antibody, anti-CD174 antibody, anti-CD227/MUC1 antibody, anti-CD352 antibody, anti-CLDN18.2 antibody, anti-DLL3 antibody, anti-ErbB2/HER2 antibody, anti-CN33 antibody, anti-GPNMB antibody, anti-ENPP3 antibody, anti-Nectin-4 antibody, anti-EGFRvIII antibody, anti-SLC44A4/AGS-5 antibody, anti-CEACAM5 antibody, anti-PSMA antibody, anti-TIM1 antibody, anti-LY6E antibody, anti-LIV1 antibody, anti-Nectin4 antibody, anti-SLITRK6 antibody, anti-HGFR/cMet antibody, anti-SLAMF7/CS1 antibody, anti-EGFR antibody, anti-BCMA antibody, anti-AXL antibody, anti-NaPi2B antibody, anti-GCC antibody, anti-STEAP1 antibody, anti-MUC16 antibody, anti-Mesothelin antibody, anti-ETBR antibody, anti-EphA2 antibody, anti-5T4 antibody, anti-FOLR1 antibody, anti-LAMP1 antibody, anti-Cadherin 6 antibody, anti-FGFR2 antibody, anti-FGFR3 antibody, anti-CA6 antibody, anti-CanAg antibody, anti-integrin αV antibody, anti-TDGF1 antibody, anti-Ephrin A4 antibody, anti-TROP2 antibody, anti-PTK7 antibody, anti-NOTCH3 antibody, anti-C4.4A antibody, anti-FLT3 antibody, anti-B7H3/4 antibody, anti-Tissue Factor antibody, or anti-ROR1/2 antibody; more preferably, anti-HER2 antibody, anti-FGFR3 antibody, anti-Trop2 antibody, anti-HER3 antibody or anti-FRα antibody; more preferably, the antibody is anti-FGFR3 antibody, anti-Trop2 antibody, anti-HER3 antibody or anti-FRα antibody;
further preferably, the antibody is an anti-human FGFR3 antibody, preferably Ab0206; or an anti-human HER2 antibody, preferably Ab0036 or Ab0001.

In a third aspect, provided is use of the conjugate of the present invention or the pharmaceutical composition thereof in the manufacture of a medicament for treating a disease; wherein the disease is a tumor or an autoimmune disease.

### BRIEF DESCRIPTION OF THE DRAWINGS

FIG. 1 shows the efficacy of the conjugates EC302-2-4-1-1 and EC301-2-1-1-1 and the corresponding payload in the NCI-N87 HER2-high cell line.

FIG. 2 shows the efficacy of the conjugates EC302-2-4-1-1 and EC301-2-1-1-1 and the corresponding payload in the SK-BR-3 HER2-high cell line.

FIG. 3 shows the ADCs have no influence on MDA-MB-468 HER2-negative cell line.

FIG. 4 shows the efficacy of the conjugates EC302-2-4-1-3, EC301-2-1-1-3, EC301-2-101-1-3 and EC302-2-104-1-3 and the corresponding payload in the NCI-N87 HER2-high cell line.

FIG. 5 shows the efficacy of the conjugates EC302-2-4-1-3, EC301-2-1-1-3, EC301-2-101-1-3 and EC302-2-104-1-3 and the corresponding payload in the SK-BR-3 HER2-high cell line.

FIG. 6 shows the ADCs have no influence on MDA-MB-468 HER2-negative cell line.

FIG. 7 shows the conjugate EC302-2-4-1-2 have no influence on MCF-7 FGFR3-negative cell line.

FIG. 8 shows the in vivo efficacy of the conjugates EC301-2-1-1-3 and EC302-2-4-1-3 and the corresponding payload in NCI-N87 CDX model.

FIG. 9 shows the body weight changes in the tests on NCI-N87 CDX model.

FIG. 10 shows the in vivo efficacy of the conjugates EC301-2-1-1-3 and EC302-2-4-1-3 and the corresponding payload in Capan-1 CDX model.

FIG. 11 shows the body weight changes in the tests on Capan-1 CDX model.

### DETAILED DESCRIPTION

The specific embodiments are provided below to illustrate technical contents of the present disclosure. Those skilled in the art can easily understand other advantages and effects of the present disclosure through the contents disclosed in the specification. The present disclosure can also be implemented or applied through other different specific embodiments. Various modifications and variations can be made by those skilled in the art without departing from the spirit of the present disclosure.

### Definitions

Unless otherwise defined hereinafter, all technical and scientific terms used herein have the same meaning as commonly understood by those skilled in the art. The techniques used herein refer to those that are generally understood in the art, including the variants and equivalent substitutions that are obvious to those skilled in the art. While the following terms are believed to be readily comprehensible by those skilled in the art, the following definitions are set forth to better illustrate the present disclosure. When a trade name is present herein, it refers to the corresponding commodity or the active ingredient thereof. All patents, published patent applications and publications cited herein are hereby incorporated by reference.

When a certain amount, concentration, or other value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range. For example, the expression “i is an integer of 1 to 20” means that i is any integer of 1 to 20, for example, i can be 1, 2, 3, 4, 5, 6, 7, 8, 9, 10, 11, 12, 13, 14, 15, 16, 17, 18, 19 or 20. Other similar expressions such as j, k and g should also be understood in a similar manner.

Unless the context clearly dictates otherwise, singular forms like “a” and “the” include the plural forms. The expression “one or more” or “at least one” may mean 1, 2, 3, 4, 5, 6, 7, 8, 9 or more.

The terms “about” and “approximately”, when used in connection with a numerical variable, generally mean that the value of the variable and all values of the variable are within experimental error (for example, within a 95% confidence interval for the mean) or within +10% of a specified value, or a wider range.

The term “stoichiometric ratio” means matching various substances according to a certain amount by weight. For example, in the present disclosure, the active ingredient is mixed with a filler, a binder, and a lubricant in a designated weight ratio.

The term “optional” or “optionally” means the event described subsequent thereto may, but not necessarily happen, and the description includes the cases wherein said event or circumstance happens or does not happen.

The expressions “comprising”, “including”, “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

The term “targeting molecule” refers to a molecule that has an affinity for a particular target (e.g., a receptor, a cell surface protein, a cytokine, a tumor specific antigen, etc.). A targeting molecule can deliver the payload to a specific site in vivo through targeted delivery. A targeting molecule can recognize one or more targets. The specific target sites are defined by the targets it recognizes. For example, a targeting molecule that targets a receptor can deliver a cytotoxin to a site containing a large number of the receptor. Examples of targeting molecules include, but are not limited to antibodies, antibody fragments, binding proteins for a given antigen, antibody mimics, scaffold proteins having affinity for a given target, ligands, and the like.

As used herein, the term “antibody” is used in a broad way and particularly includes intact monoclonal antibodies, polyclonal antibodies, monospecific antibodies, multispecific antibodies (e.g., bispecific antibodies), and antibody fragments, as long as they have the desired biological activity. The antibody may be of any subtype (such as IgG, IgE, IgM, IgD, and IgA) or subclass, and may be derived from any suitable species. In some embodiments, the antibody is of human or murine origin. The antibody may also be a fully human antibody, humanized antibody or chimeric antibody prepared by recombinant methods.

Monoclonal antibodies are used herein to refer to antibodies obtained from a substantially homogeneous population of antibodies, i.e., the individual antibodies constituting the population are identical except for a small number of possible natural mutations. Monoclonal antibodies are highly specific for a single antigenic site. The word “monoclonal” refers to that the characteristics of the antibody are derived from a substantially homogeneous population of antibodies and are not to be construed as requiring some particular methods to produce the antibody.

An intact antibody or full-length antibody essentially comprises the antigen-binding variable region(s) as well as the light chain constant region(s) (CL) and heavy chain constant region(s) (CH), which could include CH1, CH₂, CH₃ and CH₄, depending on the subtype of the antibody. An antigen-biding variable region (also known as a fragment variable region, Fv fragment) typically comprises a light chain variable region (V_{L}) and a heavy chain variable region (V_{H}). A constant region can be a constant region with a native sequence (such as a constant region with a human native sequence) or an amino acid sequence variant thereof. The variable region recognizes and interacts with the target antigen. The constant region can be recognized by and interacts with the immune system.

An antibody fragment may comprise a portion of an intact antibody, preferably its antigen binding region or variable region. Examples of antibody fragments include Fab, Fab′, F(ab′)2, Fd fragment consisting of V_{H} and CH1 domains, Fv fragment, single-domain antibody (dAb) fragment, and isolated complementarity determining region (CDR). The Fab fragment is an antibody fragment obtained by papain digestion of a full-length immunoglobulin, or a fragment having the same structure produced by, for example, recombinant expression. A Fab fragment comprises a light chain (comprising a V_{L} and a CL) and another chain, wherein the said other chain comprises a variable domain of the heavy chain (V_{H}) and a constant region domain of the heavy chain (CH1). The F(ab′)2 fragment is an antibody fragment obtained by pepsin digestion of an immunoglobulin at pH 4.0-4.5, or a fragment having the same structure produced by, for example, recombinant expression. The F(ab′)2 fragment essentially comprises two Fab fragments, wherein each heavy chain portion comprises a few additional amino acids, including the cysteines that form disulfide bonds connecting the two fragments. A Fab′ fragment is a fragment comprising one half of a F(ab′)2 fragment (one heavy chain and one light chain). The antibody fragment may comprise a plurality of chains joined together, for example, via a disulfide bond and/or via a peptide linker. Examples of antibody fragments also include single-chain Fv (scFv), Fv, dsFv, diabody, Fd and Fd′ fragments, and other fragments, including modified fragments. An antibody fragment typically comprises at least or about 50 amino acids, and typically at least or about 200 amino acids. An antigen-binding fragment can include any antibody fragment that, when inserted into an antibody framework (e.g., by substitution of the corresponding region), can result in an antibody that immunospecifically binds to the antigen.

Antibodies according to the present disclosure can be prepared using techniques well known in the art, such as the following techniques or a combination thereof: recombinant techniques, phage display techniques, synthetic techniques, or other techniques known in the art. For example, a genetically engineered recombinant antibody (or antibody mimic) can be expressed by a suitable culture system (e.g., E. coli or mammalian cells). The engineering can refer to, for example, the introduction of a ligase-specific recognition sequence at its terminals.

Specific antibodies can be listed as follows: anti-CD19 antibody, anti-CD20 antibody, anti-CD22 antibody, anti-CD25 antibody, anti-CD30/TNFRSF8 antibody, anti-CD33 antibody, anti-CD37 antibody, anti-CD44v6 antibody, anti-CD56 antibody, anti-CD70 antibody, anti-CD71 antibody, anti-CD74 antibody, anti-CD79b antibody, anti-CD117/KITk antibody, anti-CD123 antibody, anti-CD138 antibody, anti-CD142 antibody, anti-CD174 antibody, anti-CD227/MUC1 antibody, anti-CD352 antibody, anti-CLDN18.2 antibody, anti-DLL3 antibody, anti-ErbB2/HER2 antibody, anti-CN33 antibody, anti-GPNMB antibody, anti-ENPP3 antibody, anti-Nectin-4 antibody, anti-EGFRVIII antibody, anti-SLC44A4 AGS-5 antibody, anti-CEACAM5 antibody, anti-PSMA antibody, anti-TIM1 antibody, anti-LY6E antibody, anti-LIV1 antibody, anti-Nectin4 antibody, anti-SLITRK6 antibody, anti-HGFR/cMet antibody, anti-SLAMF7/CS1 antibody, anti-EGFR antibody, anti-BCMA antibody, anti-AXL antibody, anti-NaPi2B antibody, anti-GCC antibody, anti-STEAP1 antibody, anti-MUC16 antibody, anti-Mesothelin antibody, anti-ETBR antibody, anti-EphA2 antibody, anti-5T4 antibody, anti-FOLR 1 antibody, anti-LAMP1 antibody, anti-Cadherin 6 antibody, anti-FGFR2 antibody, anti-FGFR3 antibody, anti-CA6 antibody, anti-CanAg antibody, anti-integrin αV antibody, anti-TDGF1 antibody, anti-Ephrin A4 antibody, anti-TROP2 antibody, anti-PTK7 antibody, anti-NOTCH3 antibody, anti-C4.4A antibody, anti-FLT3 antibody, anti-B7H3/4 antibody, anti-Tissue Factor antibody, anti-ROR1/2 antibody, preferably, anti-HER2 antibody, anti-FGFR3 antibody, anti-Trop2 antibody, anti-HER3 antibody or anti-FRα antibody.

HER2 refers to human epidermal growth factor receptor-2, which belongs to the epidermal growth factor (EGFR) receptor tyrosine kinase family. In the present application, the terms ErbB2 and HER2 have the same meaning and can be used interchangeably.

As used herein, the term “targeting molecule-drug conjugate” is referred to as “conjugate”. Examples of conjugates include, but are not limited to, antibody-drug conjugates.

A small molecule compound refers to a molecule with a size comparable to that of an organic molecule commonly used in medicine. The term does not encompass biological macromolecules (e.g., proteins, nucleic acids, etc.), but encompasses low molecular weight peptides or derivatives thereof, such as dipeptides, tripeptides, tetrapeptides, pentapeptides, and the like. Typically, the molecular weight of the small molecule compound can be, for example, about 100 to about 2000 Da, about 200 to about 1000 Da, about 200 to about 900 Da, about 200 to about 800 Da, about 200 to about 700 Da, about 200 to about 600 Da, about 200 to about 500 Da.

Cytotoxin refers to a substance that inhibits or prevents the expression activity of a cell, cellular function, and/or causes destruction of cells. The cytotoxins currently used in ADCs are more toxic than chemotherapeutic drugs. Examples of cytotoxins include, but are not limited to, drugs that target the following targets: microtubule cytoskeleton, DNA, RNA, kinesin-mediated protein transport, regulation of apoptosis. The drug that targets microtubule cytoskeleton may be, for example, a microtubule-stabilizing agent or a tubulin polymerization inhibitor. Examples of microtubule-stabilizing agents include but are not limited to taxanes. Examples of tubulin polymerization inhibitors include but are not limited to maytansinoids, auristatins, vinblastines, colchicines, and dolastatins. The DNA-targeting drug can be, for example, a drug that directly disrupts the DNA structure or a topoisomerase inhibitor. Examples of drugs that directly disrupt DNA structure include but are not limited to DNA double strand breakers, DNA alkylating agents, DNA intercalators. The DNA double strand breakers can be, for example, an enediyne antibiotic, including but not limited to dynemicin, esperamicin, neocarzinostatin, uncialamycin, and the like. The DNA alkylating agent may be, for example, a DNA bis-alkylator (i.e. DNA-cross linker) or a DNA mono-alkylator. Examples of DNA alkylating agents include but are not limited to pyrrolo[2,1-c][1,4]benzodiazepine (PBD) dimer, 1-(chloromethyl)-2,3-dihydrogen-1H-benzo[e]indole (CBI) dimer, CBI-PBD heterodimer, dihydroindolobenzodiazepine (IGN) dimer, duocarmycin-like compound, and the like. Examples of topoisomerase inhibitors include but are not limited to exatecan and derivatives thereof (such as DX8951f, DXd-(1) and DXd-(2), the structures of which are depicted below), camptothecins and anthracyclines. The RNA-targeting drug may be, for example, a drug that inhibits splicing, and examples thereof include but are not limited to pladienolide. Drugs that target kinesin-mediated protein transport can be, for example, mitotic kinesin inhibitors including, but not limited to, kinesin spindle protein (KSP) inhibitors.

A spacer is a structure that is located between different structural modules and can spatially separate the structural modules. The definition of spacer is not limited by whether it has a certain function or whether it can be cleaved or degraded in vivo. Examples of spacers include but are not limited to amino acids and non-amino acid structures, wherein non-amino acid structures can be, but are not limited to, amino acid derivatives or analogues. “Spacer sequence” refers to an amino acid sequence serving as a spacer, and examples thereof include but are not limited to a single amino acid, a sequence containing a plurality of amino acids, for example, a sequence containing two amino acids such as GA, etc., or, for example, GGGGS, GGGGSGGGGS, GGGGSGGGGSGGGGS, etc. Self-immolative spacers are covalent assemblies tailored to correlate the cleavage of two chemical bonds after activation of a protective part in a precursor: Upon stimulation, the protective moiety (such as a cleavable sequence) is removed, which generates a cascade of disassembling reactions leading to the temporally sequential release of smaller molecules. Examples of self-immolative spacers include but not limited to PABC (p-aminobenzyloxycarbonyl, also abbreviated as PAB), acetal, heteroacetal and the combination thereof.

The term “alkyl” refers to a straight or branched saturated aliphatic hydrocarbon group consisting of carbon atoms and hydrogen atoms, which is connected to the rest of the molecule through a single bond. The alkyl group may contain 1 to 20 carbon atoms, referring to C₁-C₂₀ alkyl group, for example, C₁-C₄ alkyl group, C₁-C₃ alkyl group, C₁-C₂ alkyl, C₃ alkyl, C₄ alkyl, C₃-C₆ alkyl. Non-limiting examples of alkyl groups include but are not limited to methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethyl butyl, 1,1-dimethylbutyl, 2,3-dimethylbutyl, 1,3-dimethylbutyl or 1,2-dimethylbutyl, or their isomers. A bivalent radical refers to a group obtained from the corresponding monovalent radical by removing one hydrogen atom from a carbon atom with free valence electron(s). A bivalent radical have two connecting sites which are connected to the rest of the molecule. For example, an “alkylene” or an “alkylidene” refers to a saturated divalent hydrocarbon group, either straight or branched. Examples of alkylene groups include but are not limited to methylene (—CH₂—), ethylene (—C₂H₄—), propylene (—C₃H₆—), butylene (—C₄H₈—), pentylene (—C₅H₁₀—), hexylene (—C₆H₁₂—), 1-methylethylene (—CH(CH₃) CH₂—), 2-methylethylene (—CH₂CH(CH₃)—), methylpropylene, ethylpropylene, and the like.

As used herein, when a group is combined with another group, the connection of the groups may be linear or branched, provided that a chemically stable structure is formed. The structure formed by such a combination can be connected to other moieties of the molecule via any suitable atom in the structure, preferably via a designated chemical bond. For example, when two or more of the bivalent groups selected from: —CR¹R²—, C₁₋₁₀ alkylene, C₄₋₁₀ cycloalkylene, C₄₋₁₀ heterocyclylene and —(CO)— are combined together to form a combination, the two or more of the bivalent groups may form a linear connection with each other, such as —CR¹R²—C₁₋₁₀ alkylene-(CO)—, —CR¹R²—C₄₋₁₀ cycloalkylene-(CO)—, —CR¹R²—C₄₋₁₀ cycloalkylene-C₁₋₁₀ alkylene-(CO)—, —CR¹R²—CR^{1′}R^{2′}—(CO)—, —CR¹R²—CR^{1′}R^{2′}—CR^{1″}R^{2″}(CO)—, etc. The resulting bivalent structure can be further connected to other moieties of the molecule.

Compounds of the invention can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, nitrogen, oxygen, phosphorous, sulfur, fluorine, chlorine and iodine include, but are not limited to, ²H, ³H, ¹³C, ¹⁴C, ¹⁵N, ¹⁸O, ³²P, ³⁵S, ¹⁸F, ³⁶Cl and ¹²⁵I. In one embodiment, the payload (for example eribulin or the compound of formula (ii)) can exist in isotope-labeled or -enriched form, especially deuterated form.

As used herein, the expressions “antibody-conjugated drug” and “antibody-drug conjugate” has the same meaning.

### Compound of Formula (II)

In one aspect, provided is a compound of formula (II):

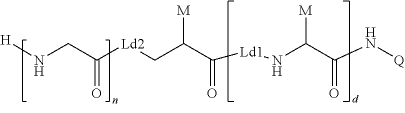

wherein,
Q is hydrogen or LKb-P;
M is hydrogen or LKa-LKb-P;
provided that Q and M are not simultaneously hydrogen;

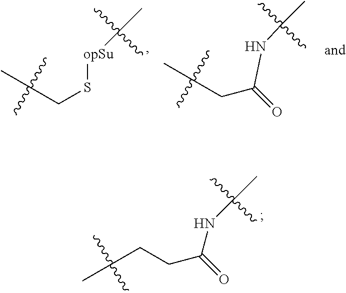

each LKa is independently selected from
opSu is

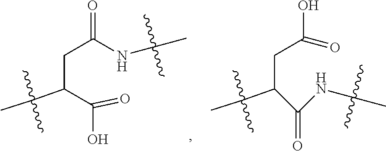 [SMILES: *NC(=O)CC(*)C(=O)O ; *NC(=O)C(*)CC(=O)O]

or a mixture thereof;
each LKb is independently L²-L¹;
P is a payload which is linked to the L¹ moiety;
L¹ is Cleavable sequence 1 comprising an amino acid sequence which can be cleaved by enzyme, and Cleavable sequence 1 comprises 1-10 amino acids;
Preferably, Cleavable sequence 1 is selected from GLy-GLy-Phe-GLy, Phe-Lys, Val-Cit, Val-Lys, GLy-Phe-Leu-Gly, Ala-Leu-Ala-Leu, Ala-Ala-Ala, Val-Cit-PABC and the combination thereof; preferably, Cleavable sequence 1 is GLy-GLy-Phe-Gly or Val-Cit-PABC;
L² is a bond; or a C₂₋₂₀ alkylene wherein one or more —CH₂— structures in the alkylene is optionally replaced by —CR¹R²—, —O—, —(CO)—, —S(═O)₂—, —NR₃—, —N+R⁴R⁵—, C₄₋₁₀ cycloalkylene, C₄₋₁₀ heterocyclylene, phenylene; wherein the cycloalkylene, heterocyclylene and phenylene are each independently unsubstituted or substituted with at least one substituent selected from halogen, —C₁₋₁₀ alkyl, —C₁₋₁₀ haloalkyl, —C₁₋₁₀ alkylene-NH—R⁶ and —C₁₋₁₀ alkylene-O—R⁷;
Ld2 and each Ld1 are independently a bond; or selected from —NH—C₁₋₂₀ alkylene-(CO)—, —NH-(PEG)ᵢ-(CO)—, or is a natural amino acid or oligomeric natural amino acids having a degree of polymerization of 2-10 independently unsubstituted or substituted with -(PEG)ⱼ-R⁸ on the side chain;
—(PEG)ᵢ- and -(PEG)ⱼ- are each a PEG fragment, which comprises the denoted number of consecutive —(O—C₂H₄)— structure units or consecutive —(C₂H₄—O)— structure units, with an optional additional C₁₋₁₀ alkylene at one terminal;
R¹, R², R³, R⁴, R⁵, R⁶, R⁷ are each independently selected from hydrogen, halogen, —C₁₋₁₀ alkyl, —C₁₋₁₀ haloalkyl, C₄₋₁₀ cycloalkylene;
R⁸ is C₁₋₁₀ alkyl;
n is any integer of 2 to 20;
d is 0, or is any integer of 1 to 6;
each i is independently an integer of 1-100, preferably 1 to 20; preferably each i is independently an integer of 1 to 12; more preferably 2 to 8; particularly 4;
each j is independently an integer of 1-100, preferably 1 to 20; preferably each j is independently an integer of 1 to 12; more preferably 8 to 12; particularly 8 or 12.

In one embodiment, L² is selected from: —(CH₂)ₚ—(CH₂)₂(CO)—, p is 0, or an integer of 1 to 5;

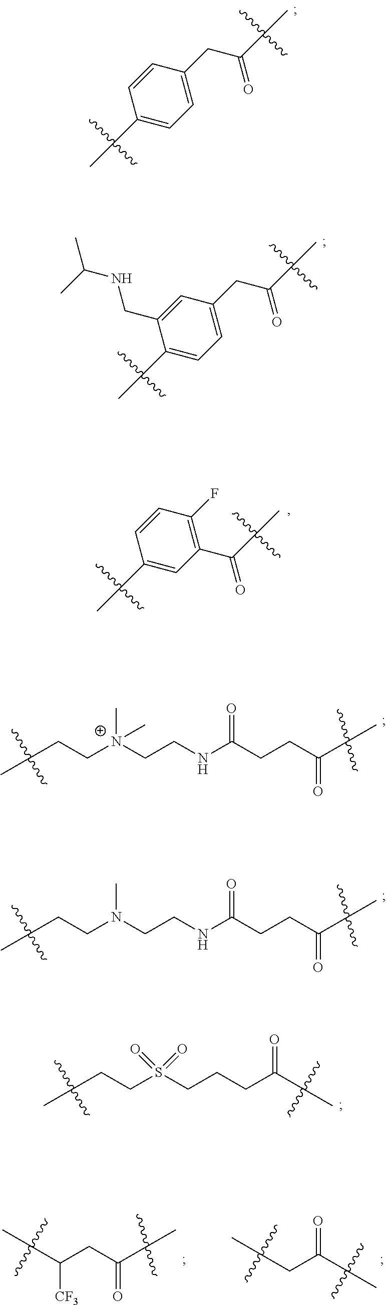

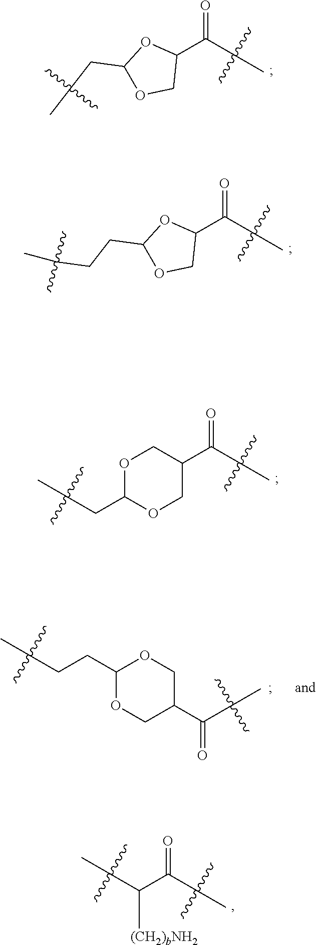

b is an integer of 1 to 10.

In one embodiment, the carbonyl group in each of the above structure of L² is connected to L¹, and the other linking site is connected to opSu.

In one embodiment, p is 0 to 3; preferably 3.
Ld2 and each Ld1 are independently a bond or

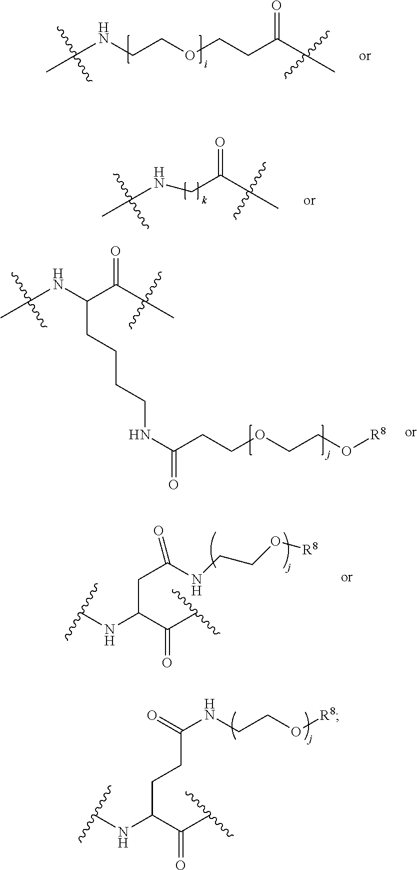

each i, j and k are independently an integer of 1-100.

In one embodiment, each i, j and k are independently an integer of 1 to 20. In one embodiment, each i, j and k are independently an integer of 1 to 12.

In one embodiment, each i is independently an integer of 2 to 8; particularly 4.

In one embodiment, each j is independently an integer of 8 to 12; particularly 8 or 12.

In one embodiment, each k is independently an integer of 1 to 7; particularly 1, or 3 or 5.

In one embodiment, Ld2 and each Ld1 are independently a bond; or a C₁₋₂₀ alkylene with an amino and a carbonyl at the two terminals respectively, or a PEG fragment of a certain length (denoted as -(PEG)ᵢ-) with an amino and a carbonyl at the two terminals respectively, or one or more natural amino acids independently unsubstituted or substituted with a PEG fragment of a certain length (denoted as -(PEG)ⱼ-) on the side chain.

In one embodiment, -(PEG)ᵢ- comprises —(O)—C₂H₄)ᵢ— or —(C₂H₄—O)ᵢ—, and an optional additional C₁₋₁₀ alkylene at one terminal; -(PEG)ⱼ-, comprises —(O—C₂H₄)ⱼ— or —(C₂H₄—O)ⱼ—, and an optional additional C₁₋₁₀ alkylene at one terminal. In a very specific -(PEG)ᵢ- comprises —C₂H₄—(O—C₂H₄)ᵢ— or —(C₂H₄—O)ᵢ—C₂H₄—,

It is to be understood that when there are two or more Ld1, L² or L¹ structures in the molecule, the structure of each Ld1, L² or L¹ is selected independently. When there are two or more R^{x} (x being 1, 2, 3, 4, 5, 6, 7, 8, 9, etc.) in the molecule, each R^{x} is selected independently. In some embodiments, the “x”s in the molecule are denoted with or without additional apostrophe (′) or apostrophes (such as ″, ″′, ″″, etc.), for example R, R^{1′}, R^{1″}, R^{1″′}, R^{2′}, R^{2″}, R^{2″′}, etc, wherein each R^{x}, with or without additional apostrophe or apostrophes, are selected independently. The other R^{x}s such as R³, R⁴, R⁵, R⁶, R⁷, R⁸, R⁹, and “Ld1” s, “L²”s and “L¹”s should be understood in a similar way. In some embodiments, the “i”s in the molecule are denoted with or without additional numbers, for example i1, i2, i3, i4, etc., wherein the numbers do not indicate any sequence, but are used merely to differentiate the “i”s. And each “i”s, with or without additional numbers, are selected independently.

In one embodiment, Cleavable sequence 1 is selected from Gly-Gly-Phe-Gly, Phe-Lys, Val-Cit, Val-Lys, Gly-Phe-Leu-Gly, Ala-Leu-Ala-Leu, Ala-Ala-Ala, Val-Cit-PABC and the combination thereof; preferably, Cleavable sequence 1 is Gly-Gly-Phe-Gly or Val-Cit-PABC. In another embodiment, Cleavable sequence 1 is Gly-Gly-Phe-Gly.

In one embodiment, Q is hydrogen.

In one embodiment, R⁸ is C₁₋₆ alkyl, preferably methyl.

In one embodiment, n is an integer of 2 to 5, especially 3.

In one embodiment, d is 0, or is any integer of 1 to 4; preferably 0, 1, 2 or 3.

Thiosuccinimide is unstable under physiological conditions and is liable to reverse Michael addition which leads to cleavage at the conjugation site. Moreover, when another thiol compound is present in the system, thiosuccinimide may also undergo thiol exchange with the other thiol compound. Both of these reactions cause the fall-off of the payload and result in toxic side effects. In the present disclosure, when applied in the linker-payload compound, the ring-opened succinimide structure no longer undergoes reverse Michael addition or thiol exchange, and thus the product is more stable. Method of ring opening reaction can be found in WO2015165413A1.

The compound comprising ring-opened succinimide moiety can be purified by semi-preparative/preparative HPLC or other suitable separation means to obtain with high purity and defined composition, regardless of the efficiency of the succinimide ring opening reaction.

### Moiety Comprising Recognition Sequence of the Ligase Acceptor or Donor Substrate

In one embodiment, the Gₙ moiety of the compound of formula (II) is a recognition sequence of a ligase acceptor or donor substrate, which facilitates enzyme-catalyzed coupling of compound of formula (II) with the targeting molecule under the catalysis of the ligase. The targeting molecule optionally modified and comprises the corresponding recognition sequence of a ligase acceptor or donor substrate.

In one embodiment, the ligase is a transpeptidase. In one embodiment, the ligase is selected from the group consisting of a natural transpeptidase, an unnatural transpeptidase, variants thereof, and the combination thereof. Unnatural transpeptidase enzymes can be, but are not limited to, those obtained by engineering of natural transpeptidase. In a preferred embodiment, the ligase is selected from the group consisting of a natural Sortase, an unnatural Sortase, and the combination thereof. The species of natural Sortase include Sortase A, Sortase B, Sortase C, Sortase D, Sortase L. plantarum, etc. (US20110321183A1). The type of ligase corresponds to the ligase recognition sequence and is thereby used to achieve specific conjugation between different molecules or structural fragments.

In some embodiments, the ligase is a Sortase selected from Sortase A, Sortase B, Sortase C, Sortase D and Sortase L. plantarum. In these embodiments, the recognition sequence of the ligase acceptor substrate is selected from the group consisting of oligomeric glycine, oligomeric alanine, and a mixture of oligomeric glycine/alanine having a degree of polymerization of 3-10. In a particular embodiment, the recognition sequence of the ligase acceptor substrate is Gₙ, wherein G is glycine (Gly), and n is an integer of 2 to 10.

In another particular embodiment, the ligase is Sortase A from Staphylococcus aureus. Accordingly, the ligase recognition sequence may be the typical recognition sequence LPXTG of the enzyme. In yet another particular embodiment, the recognition sequence of the ligase donor substrate is LPXTGJ, and the recognition sequence of the ligase acceptor substrate is Gₙ, wherein X can be any single amino acid that is natural or unnatural; J is absent, or is an amino acid fragment comprising 1-10 amino acids, optionally labeled. In one embodiment, J is absent. In yet another embodiment, J is an amino acid fragment comprising 1-10 amino acids, wherein each amino acid is independently any natural or unnatural amino acid. In another embodiment, J is Gₘ, wherein m is an integer of 1 to 10. In yet another particular embodiment, the recognition sequence of the ligase donor substrate is LPETG. In another particular embodiment, the recognition sequence of the ligase donor substrate is LPETGG.

In one embodiment, the ligase is Sortase B from Staphylococcus aureus and the corresponding donor substrate recognition sequence can be NPQTN. In another embodiment, the ligase is Sortase B from Bacillus anthracis and the corresponding donor substrate recognition sequence can be NPKTG.

In yet another embodiment, the ligase is Sortase A from Streptococcus pyogenes and the corresponding donor substrate recognition sequence can be LPXTGJ, wherein J is as defined above. In another embodiment, the ligase is Sortase subfamily 5 from Streptomyces coelicolor, and the corresponding donor substrate recognition sequence can be LAXTG.

In yet another embodiment, the ligase is Sortase A from Lactobacillus plantarum and the corresponding donor substrate recognition sequence can be LPQTSEQ.

The ligase recognition sequence can also be other totally new recognition sequence for transpeptidase optimized by manual screening.

### Payload

In the present disclosure, the payload may be selected from the group consisting of small molecule compounds, nucleic acids and analogues, tracer molecules (including fluorescent molecules, etc.), short peptides, polypeptides, peptidomimetics, and proteins. In one embodiment, the payload is selected from the group consisting of small molecule compounds, nucleic acid molecules, and tracer molecules. In a preferred embodiment, the payload is selected from small molecule compounds. In a more preferred embodiment, the payload is selected from the group consisting of cytotoxin and fragments thereof.

In one embodiment, the cytotoxin is selected from the group consisting of drugs that target microtubule cytoskeleton. In a preferred embodiment, the cytotoxin is selected from the group consisting of taxancs, maytansinoids, auristatins, epothilones, combretastatin A-4 phosphate, combretastatin A-4 and derivatives thereof, indol-sulfonamides, vinblastines such as vinblastine, vincristine, vindesine, vinorelbine, vinflunine, vinglycinate, anhy-drovinblastine, dolastatin 10 and analogues, halichondrin B and eribulin, indole-3-oxoacetamide, podophyllotoxins, 7-diethylamino-3-(2′-benzoxazolyl)-coumarin (DBC), discodermolide, laulimalide. In another embodiment, the cytotoxin is selected from the group consisting of DNA topoisomerase inhibitors such as camptothecins and derivatives thereof, mitoxantrone, mitoguazone. In a preferred embodiment, the cytotoxin is selected from the group consisting of nitrogen mustards such as chlorambucil, chlornaphazine, cholophosphamide, estramustine, ifosfamide, mechlorethamine, mechlorethamine oxide hydrochloride, melphalan, novembichin, phenamet, phenesterine, prednimustine, trofosfamide, uracil mustard. In yet another preferred embodiment, the cytotoxin is selected from the group consisting of nitrosourcas such as carmustine, flubenzuron, formoterol, lomustine, nimustine, ramustine. In one embodiment, the cytotoxin is selected from the group consisting of aziridines. In a preferred embodiment, the cytotoxin is selected from the group consisting of benzodopa, carboquone, meturedepa, and uredepa. In one embodiment, the cytotoxin is selected from the group consisting of an anti-tumor antibiotic. In a preferred embodiment, the cytotoxin is selected from the group consisting of enediyne antibiotics. In a more preferred embodiment, the cytotoxin is selected from the group consisting of dynemicin, esperamicin, neocarzinostatin, and aclacinomycin. In another preferred embodiment, the cytotoxin is selected from the group consisting of actinomycin, antramycin, bleomycins, actinomycin C, carabicin, carminomycin, and cardinophyllin, carminomycin, actinomycin D, daunorubicin, detorubicin, adriamycin, epirubicin, esorubicin, idarubicin, marcellomycin, mitomycins, nogalamycin, olivomycin, peplomycin, porfiromycin, puromycin, ferric adriamycin, rodorubicin, rufocromomycin, streptozocin, zinostatin, zorubicin. In yet another preferred embodiment, the cytotoxin is selected from the group consisting of trichothecene. In a more preferred embodiment, the cytotoxin is selected from the group consisting of T-2 toxin, verracurin A, bacillocporin A, and anguidine. In one embodiment, the cytotoxin is selected from the group consisting of an anti-tumor amino acid derivatives. In a preferred embodiment, the cytotoxin is selected from the group consisting of ubenimex, azaserine, 6-diazo-5-oxo-L-norleucine. In another embodiment, the cytotoxin is selected from the group consisting of folic acid analogues. In a preferred embodiment, the cytotoxin is selected from the group consisting of dimethyl folic acid, methotrexate, pteropterin, trimetrexate, and edatrexate. In one embodiment, the cytotoxin is selected from the group consisting of purine analogues. In a preferred embodiment, the cytotoxin is selected from the group consisting of fludarabine, 6-mercaptopurine, tiamiprine, thioguanine. In yet another embodiment, the cytotoxin is selected from pyrimidine analogues. In a preferred embodiment, the cytotoxin is selected from the group consisting of ancitabine, gemcitabine, enocitabine, azacitidine, 6-azauridine, carmofur, cytarabine, dideoxyuridine, doxifluridine, floxuridine. In one embodiment, the cytotoxin is selected from the group consisting of androgens. In a preferred embodiment, the cytotoxin is selected from the group consisting of calusterone, dromostanolone propionate, epitiostanol, mepitiostane, testolactone. In another embodiment, the cytotoxin is selected from the group consisting of anti-adrenals. In a preferred embodiment, the cytotoxin is selected from the group consisting of aminoglutethimide, mitotane, and trilostane. In one embodiment, the cytotoxin is selected from the group consisting of anti-androgens. In a preferred embodiment, the cytotoxin is selected from the group consisting of flutamide, nilutamide, bicalutamide, leuprorelin acetate, and goserelin. In yet another embodiment, the cytotoxin is selected from the group consisting of a protein kinase inhibitor and a proteasome inhibitor. In another embodiment, the cytotoxin is selected from the group consisting of vinblastines, colchicines, taxanes, auristatins, maytansinoids, calicheamicin, doxonubicin, duocarmucin, SN-38, cryptophycin analogue, deruxtecan, duocarmazine, calicheamicin, centanamycin, dolastansine, and pyrrolobenzodiazepine (PBD). In a particular embodiment, the cytotoxin is selected from the group consisting of vinblastines, colchicines, taxanes, auristatins, and maytansinoids.

In a particular embodiment, the cytotoxin is exatecan or a derivative thereof, such as DX8951f and the like. In a particular embodiment, the cytotoxin is cribulin.

The payload connects to the rest of the molecule through a single bond. In one embodiment, such bond is an amide bond or an ester bond or an ether bond. One skilled in the art can easily understand the means for a particular payload to connect to the rest of the molecule, and if needed, thereby conceive proper preparation method(s) for the compound of formula (II) comprising the particular payload.

In a particular embodiment, the compound of formula (II) can be prepared by reacting a payload compound with a linker compound, each comprising a suitable reactive group, and thus covalently conjugate the payload compound with the linker compound. For this purpose, the above-mentioned payload compounds that do not contain reactive groups require appropriate derivatization to bear proper reactive group(s). Such derivatization can also be easily understood by one skilled in the art. In one embodiment, the compound of formula (II) can be prepared by reacting a payload compound with a linker compound using any reaction known in the art, including but not limit to condensation reaction, nucleophilic addition, electrophilic addition, etc.

In one embodiment, in the compound of formula (II), P has the structure of formula (i)

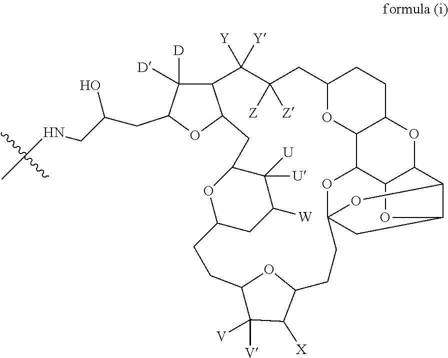

wherein,
each of D and D′ is independently selected from R⁹ and OR⁹, wherein R⁹ is H, C₁₋₃ alkyl, or C₁₋₃ haloalkyl;
each of U and U′ is independently H, C₁₋₃ alkoxy, or C₁₋₃ alkyl; or U and U′ taken together are ═CH₂;
W is C₁₋₃ alkyl;
each of V and V′ is independently H, C₁₋₃ alkoxy, or C₁₋₃ alkyl; or U and U′ taken together are ═CH₂;
X is H or C₁₋₆ alkoxy;
each of Y and Y′ is independently H or C₁₋₃ alkoxy; or Y and Y′ taken together are ═O, ═CH₂; and
each of Z and Z′ is independently H or C₁₋₃ alkoxy; or Z and Z′ taken together are ═O, ═CH₂;
or a pharmaceutically acceptable salt thereof.

In a more preferred embodiment, P has the structure of formula (i-1)

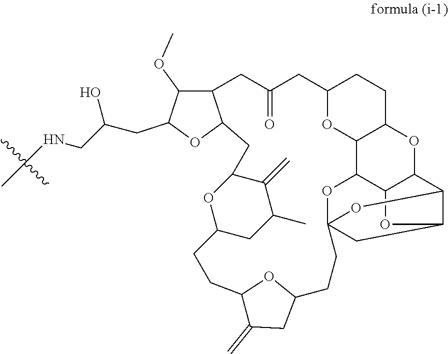 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]

In a further preferred embodiment, P has the structure of formula (ii)

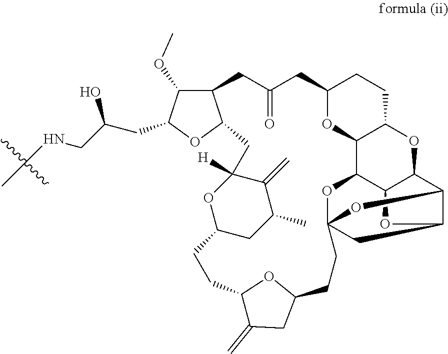 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

wherein the wavy bond shows the connection site for connection to the L¹ moiety.

In one embodiment, the compound of formula (II) has the structure of formula (II-i)

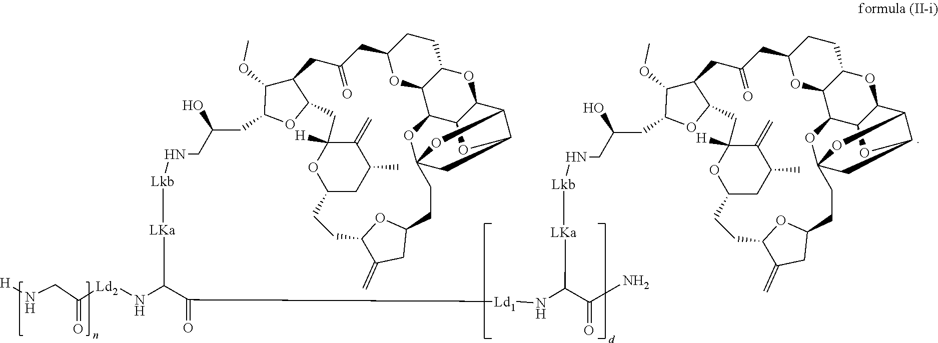

### Specific Embodiment of the Formula (II) Compound

In one embodiment, Q is hydrogen, each LKa is

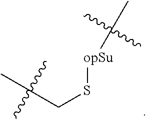

In one embodiment, formula (II) has the structure of formula (II-1)

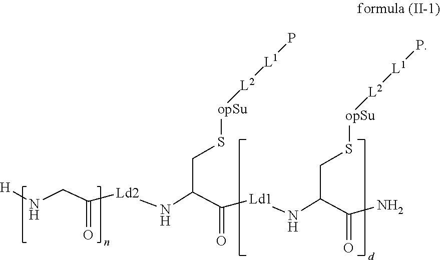

In one embodiment, Ld2 is a bond, d is 0. In one embodiment, the compound of formula (II-1) is as follows:

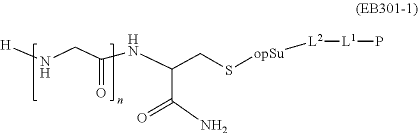

In one embodiment, d is 0, Ld2 is

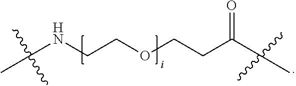

In one embodiment, the compound of formula (II-1) is as follows:

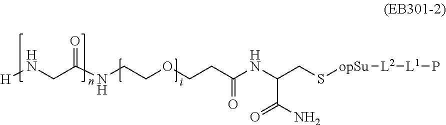

In one embodiment, d is 1, 2 or 3, Ld2 and each Ld1 are independently selected from

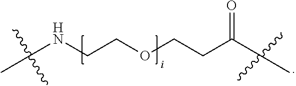

In one embodiment, the compound of formula (II-1) is as follows:

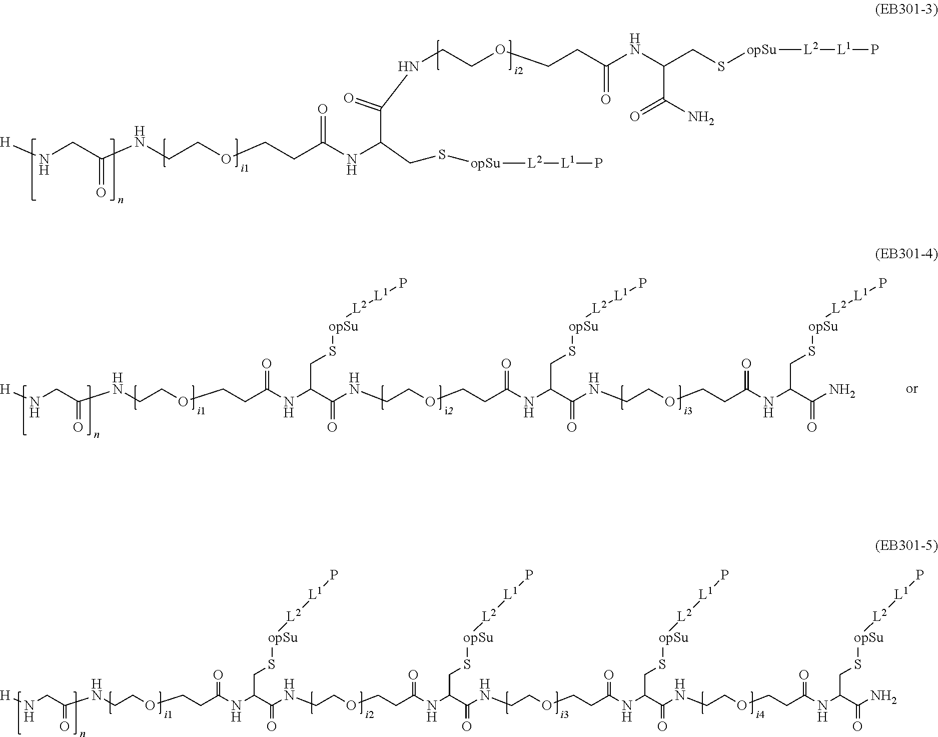

In one embodiment, Ld2 is

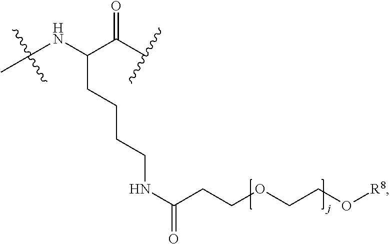

d is 0. In one embodiment, the compound of formula (II-1) is as follows:

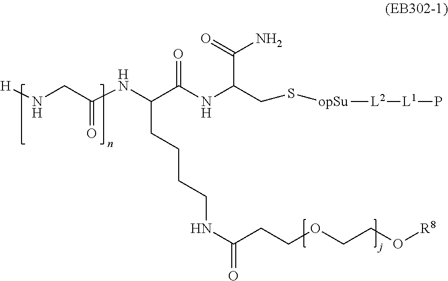

In one embodiment, d is 1, 2 or 3, Ld2 is

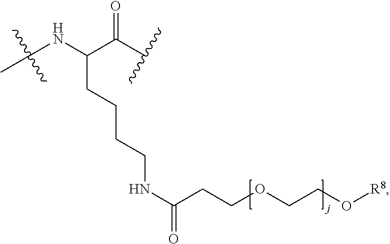

and each Ld1 is independently selected from

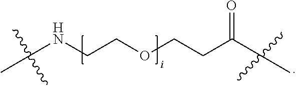

In one embodiment, the compound of formula (II-1) is as follows:

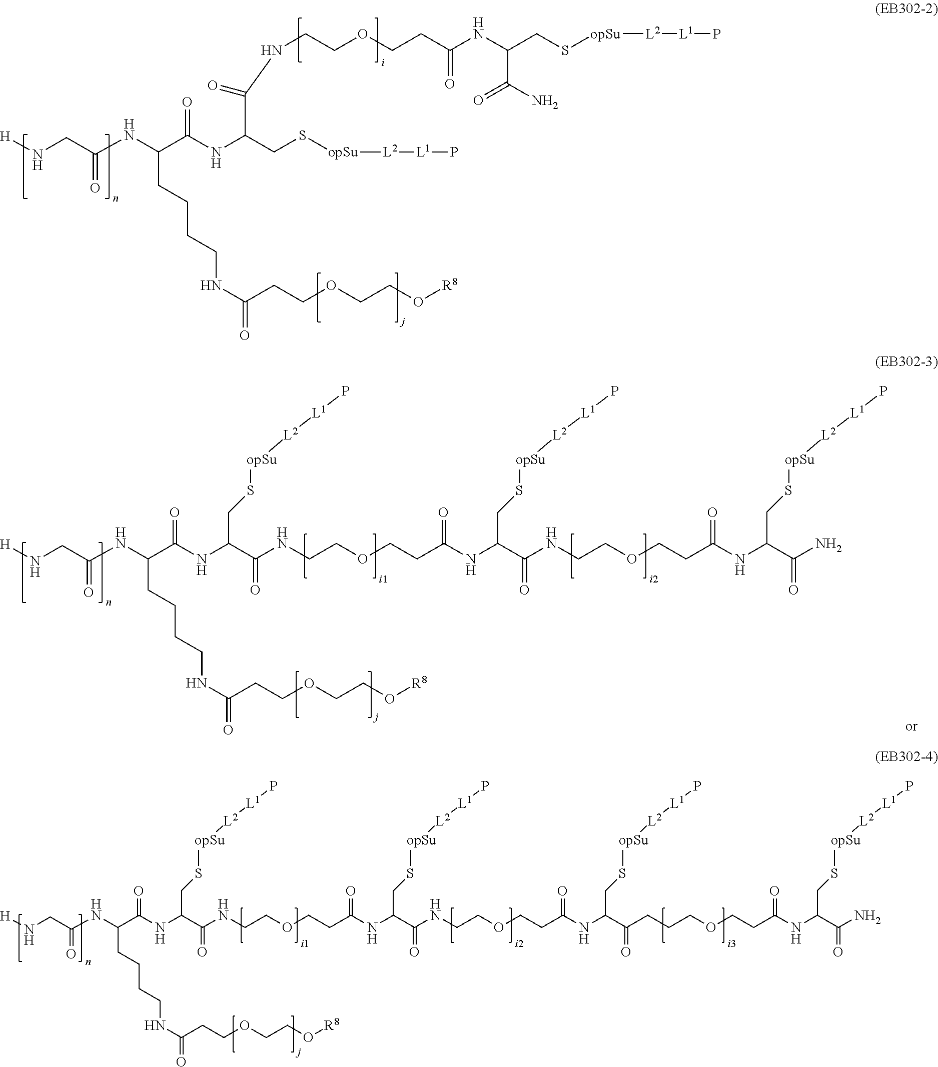

In one embodiment, n is 3, L² is —(CH₂)ₚ—(CH₂)₂(CO)—, p is 3, L¹ is GGFG.

In one embodiment, EB301-1 has the structure of EB301-1-1:

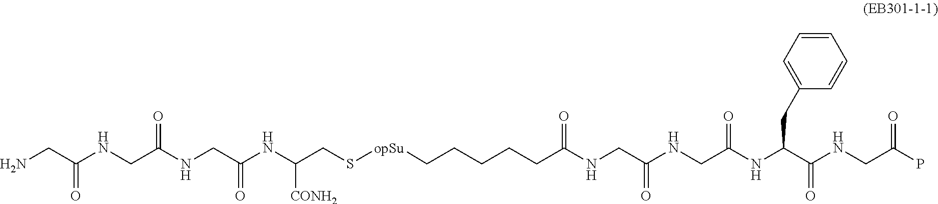

In one embodiment, EB301-2 has the structure of EB301-2-0:

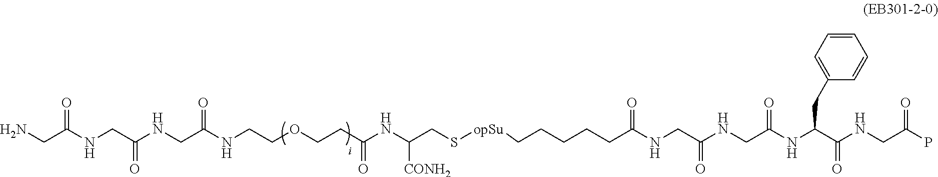

In one embodiment, EB301-3 has the structure of EB301-3-0:

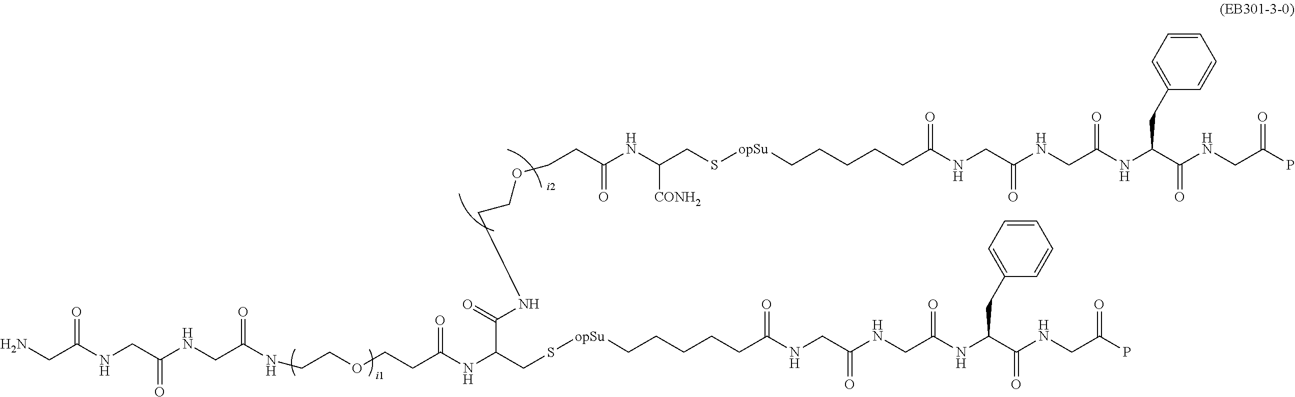

In one embodiment, EB301-4 has the structure of EB301-4-0:

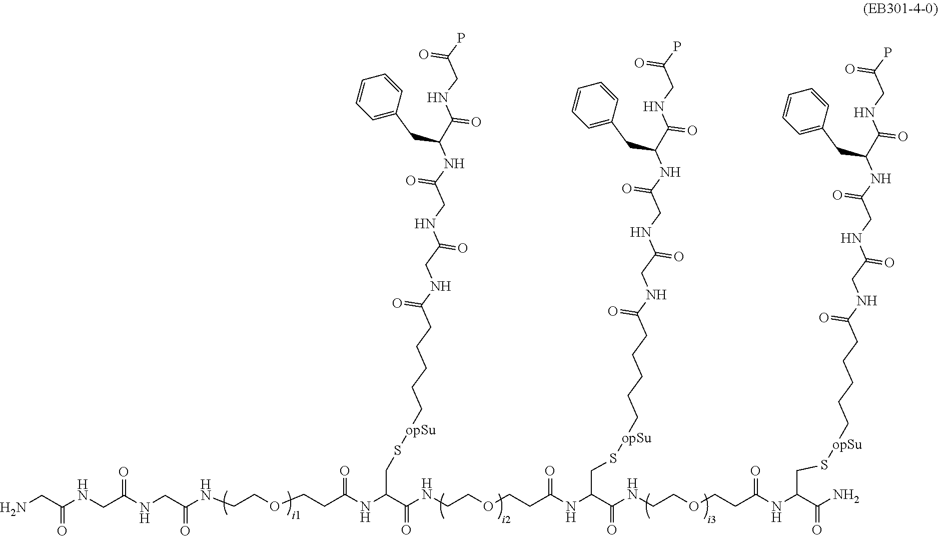

In one embodiment, EB301-5 has the structure of EB301-5-0:

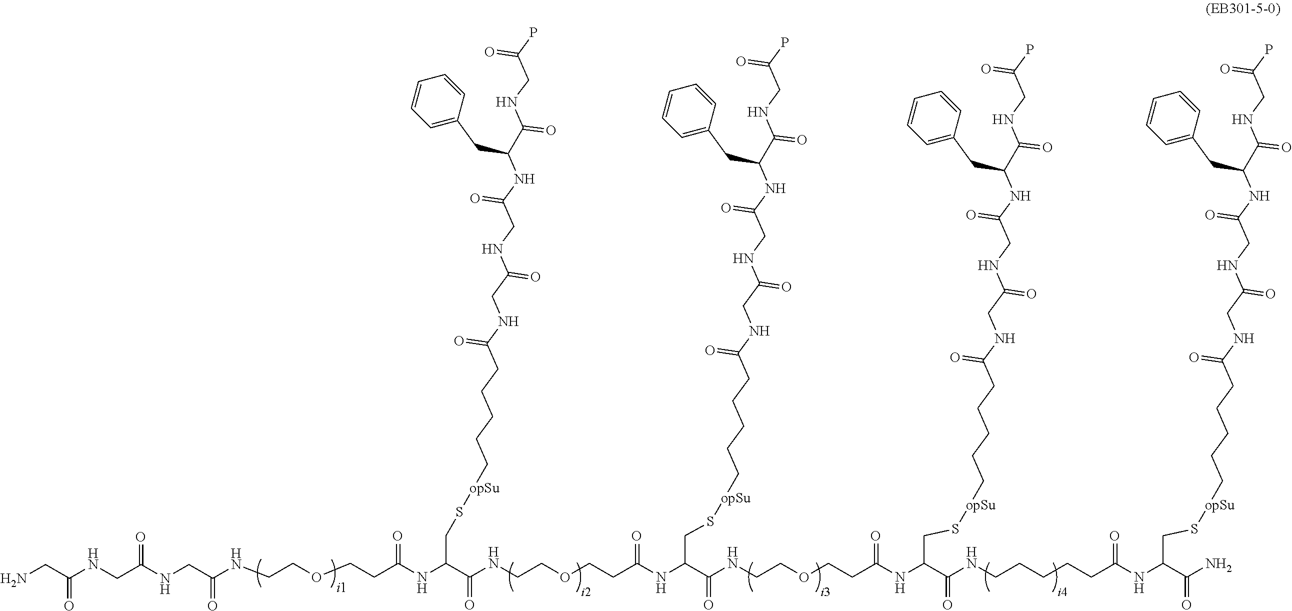

In one embodiment, EB302-1 has the structure of EB302-1-0:

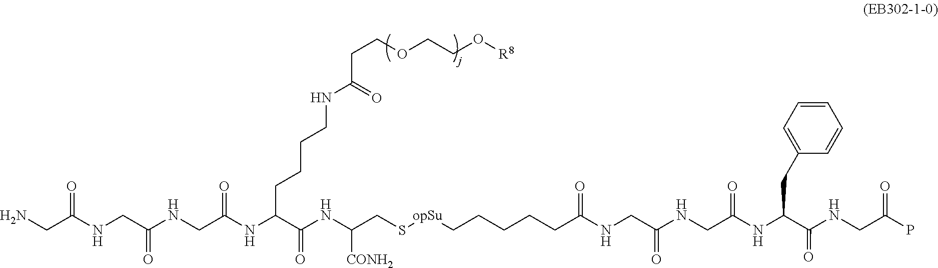

In one embodiment, EB302-2 has the structure of EB302-2-0:

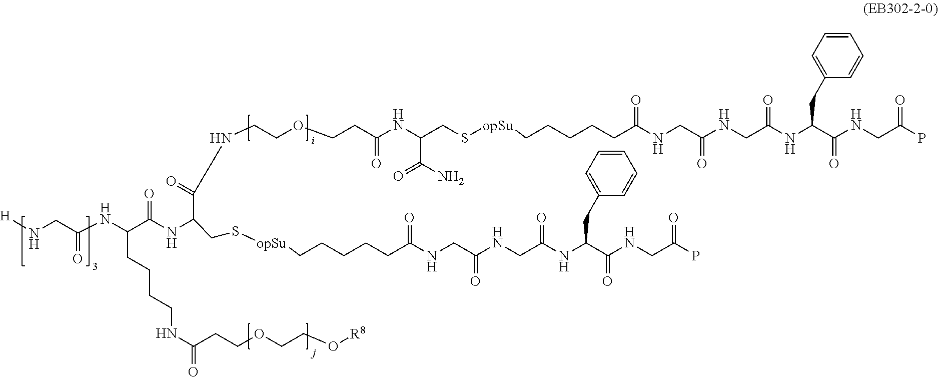

In one embodiment, EB302-3 has the structure of EB302-3-0:

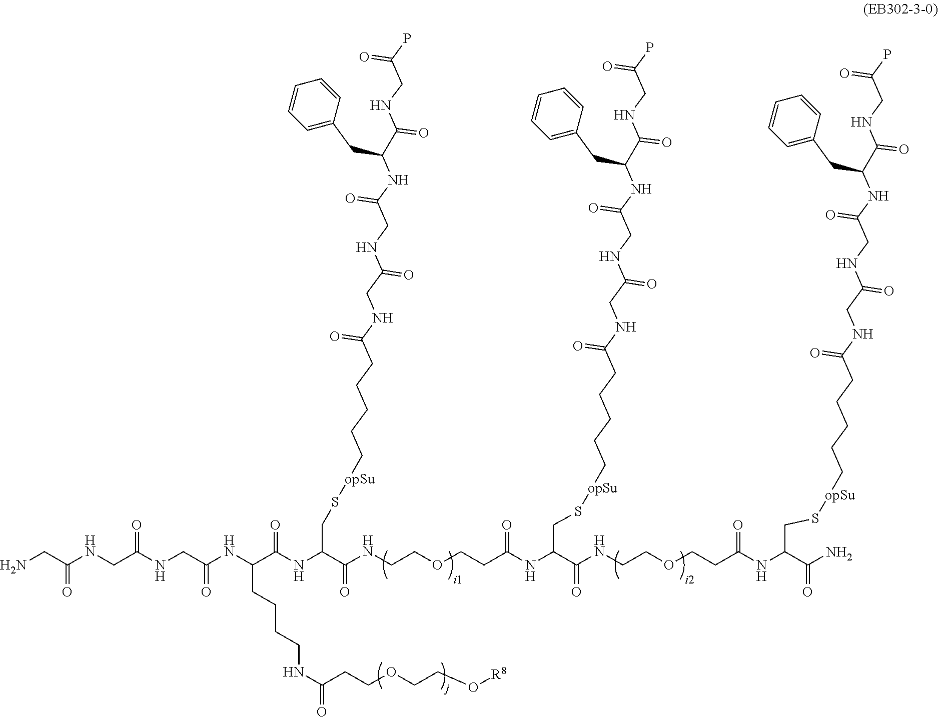

In one embodiment, EB302-4 has the structure of EB302-4-0:

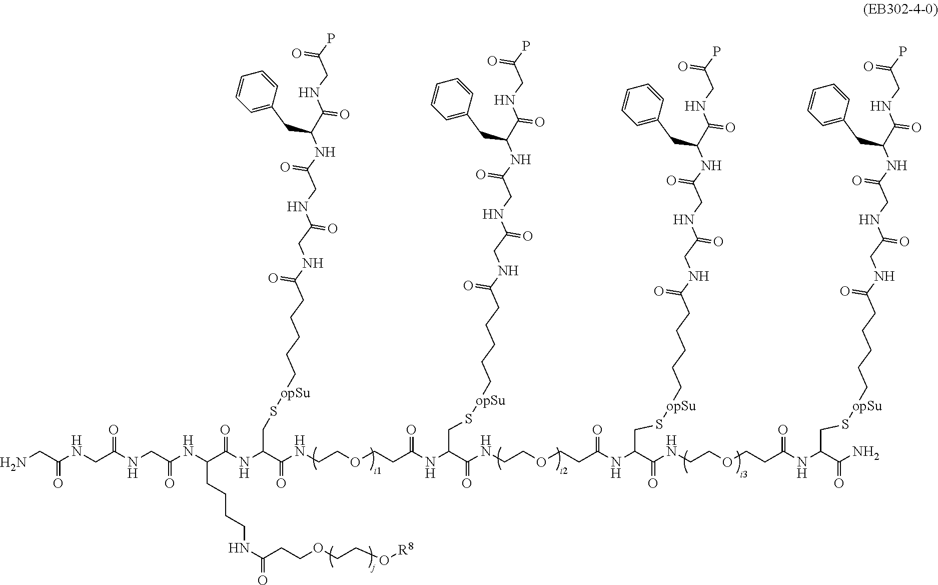

In an embodiment, i is 4. In one embodiment, EB301-2-0 has the structure of EB301-2-1:

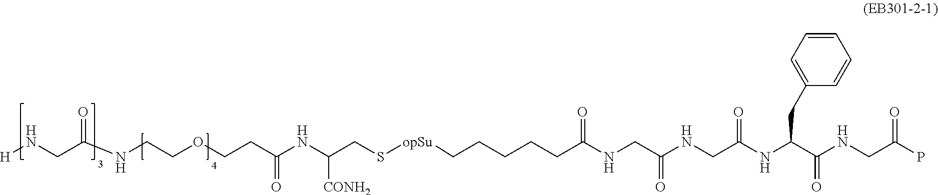

In an embodiment, i is 4, j is 8. In an embodiment, EB302-2-0 has the structure of EB302-2-1:

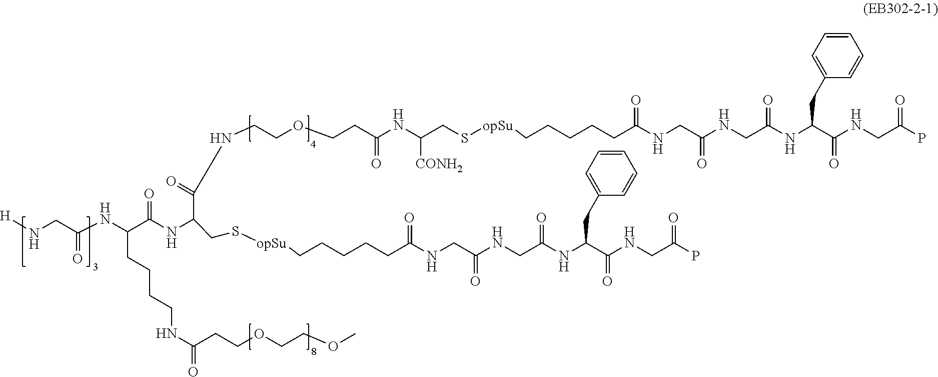

In an embodiment, i is 4, j is 12. In an embodiment, EB302-2-0 has the structure of EB302-2-4:

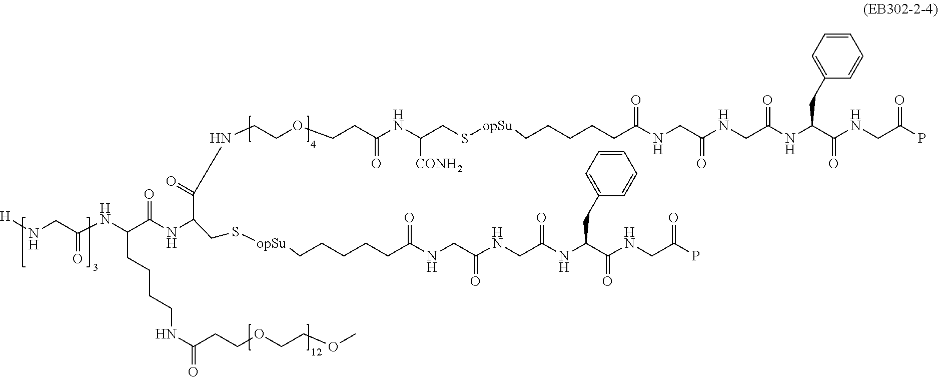

In one embodiment, n is 3, L² is —(CH₂)—(CH₂)> (CO)—, p is 3, L¹ is Val-Cit-PABC.

In one embodiment, EB301-1 has the structure of EB301-1-101:

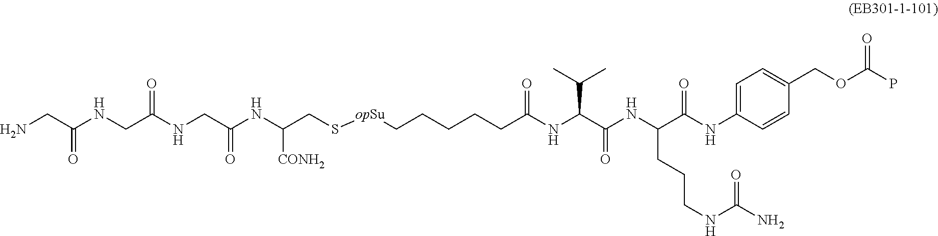

In one embodiment, EB301-2 has the structure of EB301-2-100:

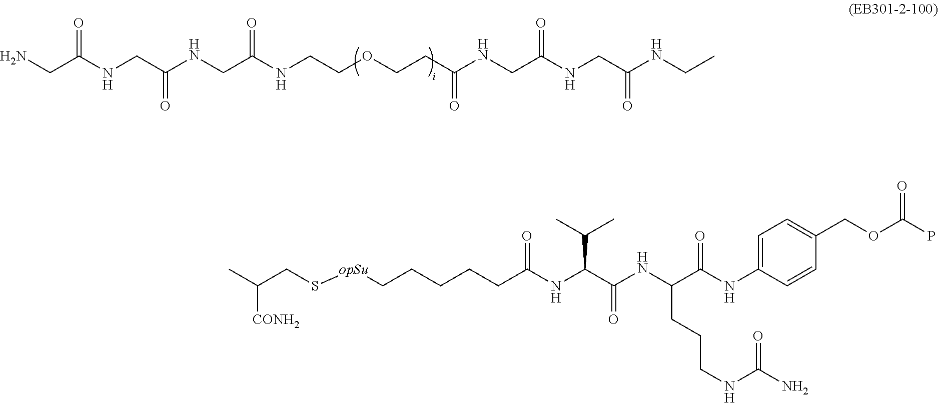

In one embodiment, EB301-3 has the structure of EB301-3-100:

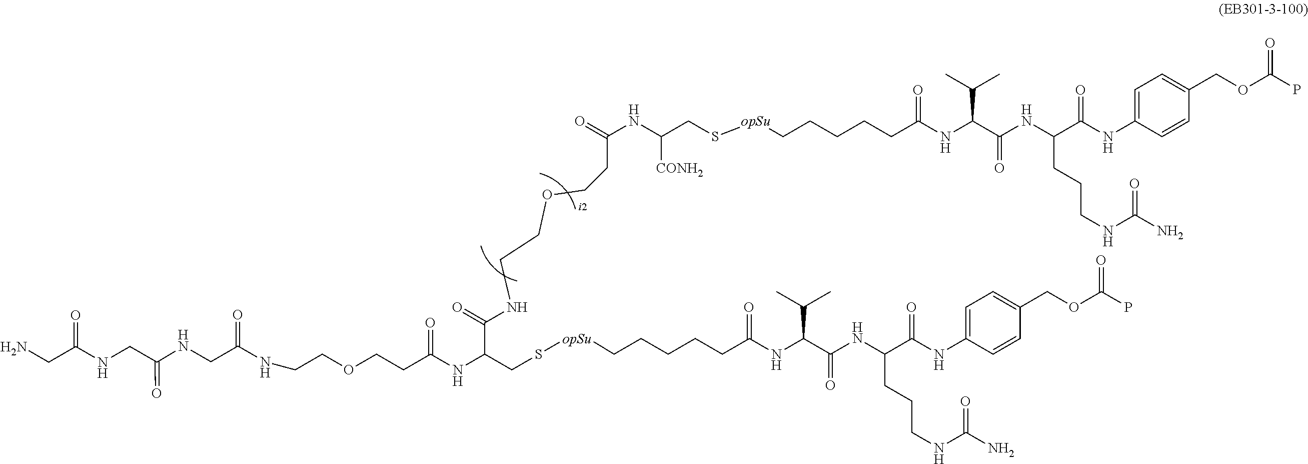

In one embodiment, EB301-4 has the structure of EB301-4-100:

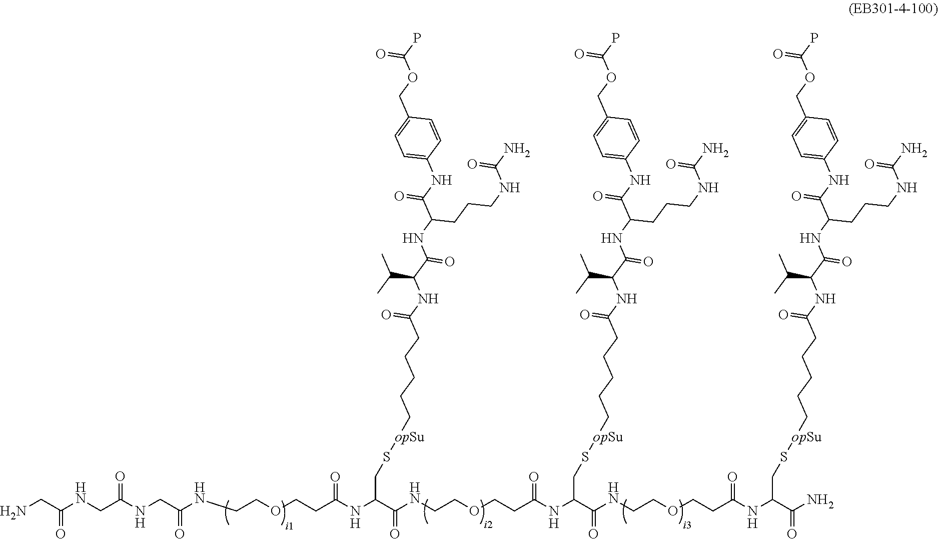

In one embodiment, EB301-5 has the structure of EB301-5-100:

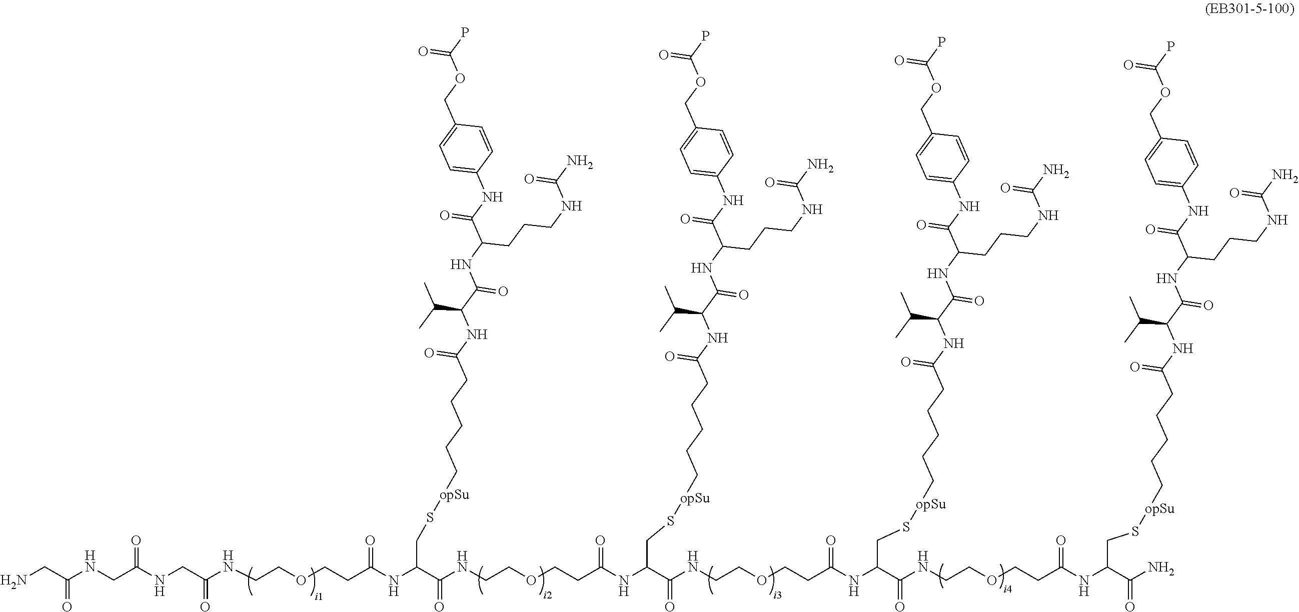

In one embodiment, EB302-1 has the structure of EB302-1-100:

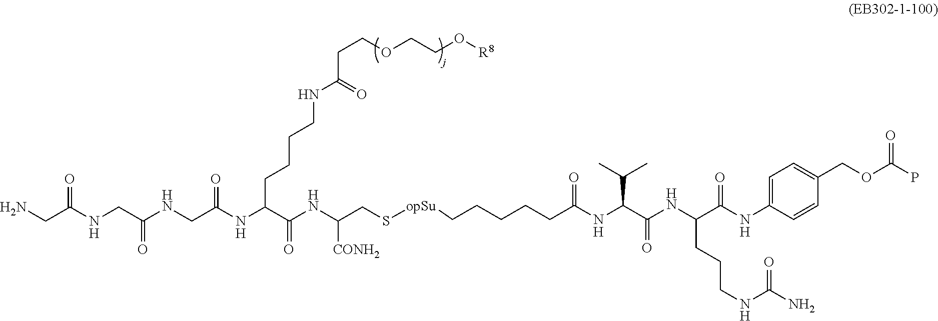

In one embodiment, EB302-2 has the structure of EB302-2-100:

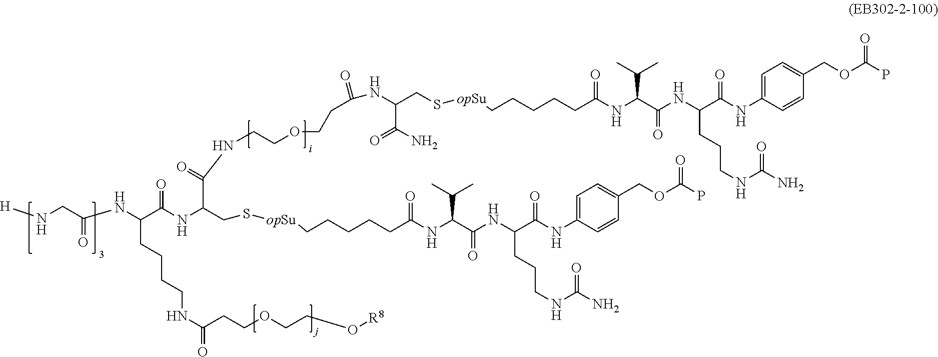

In one embodiment, EB302-3 has the structure of EB302-3-100:

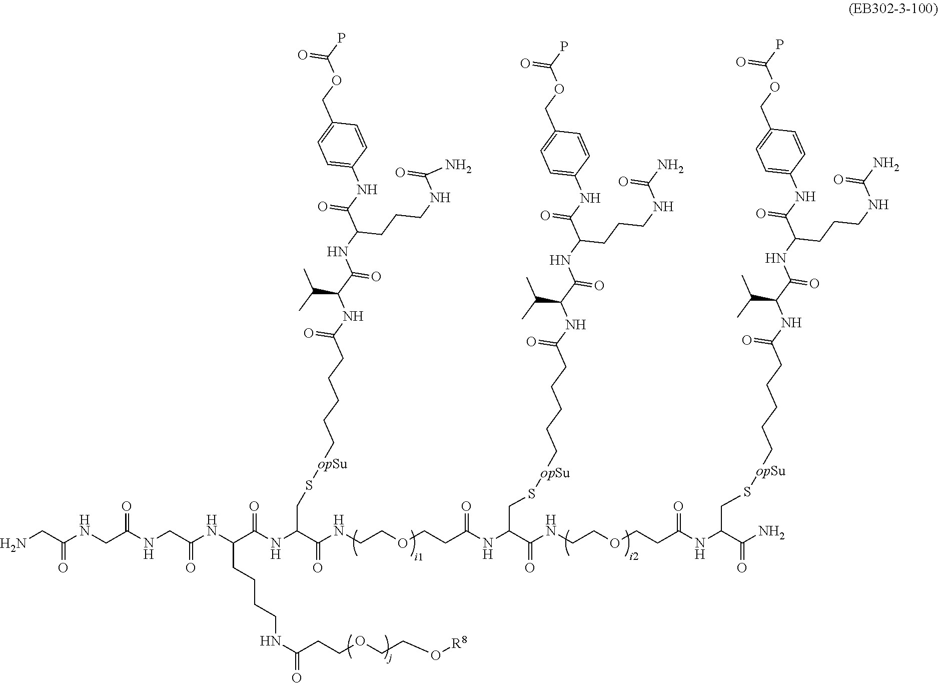

In one embodiment, EB302-4 has the structure of EB302-4-100:

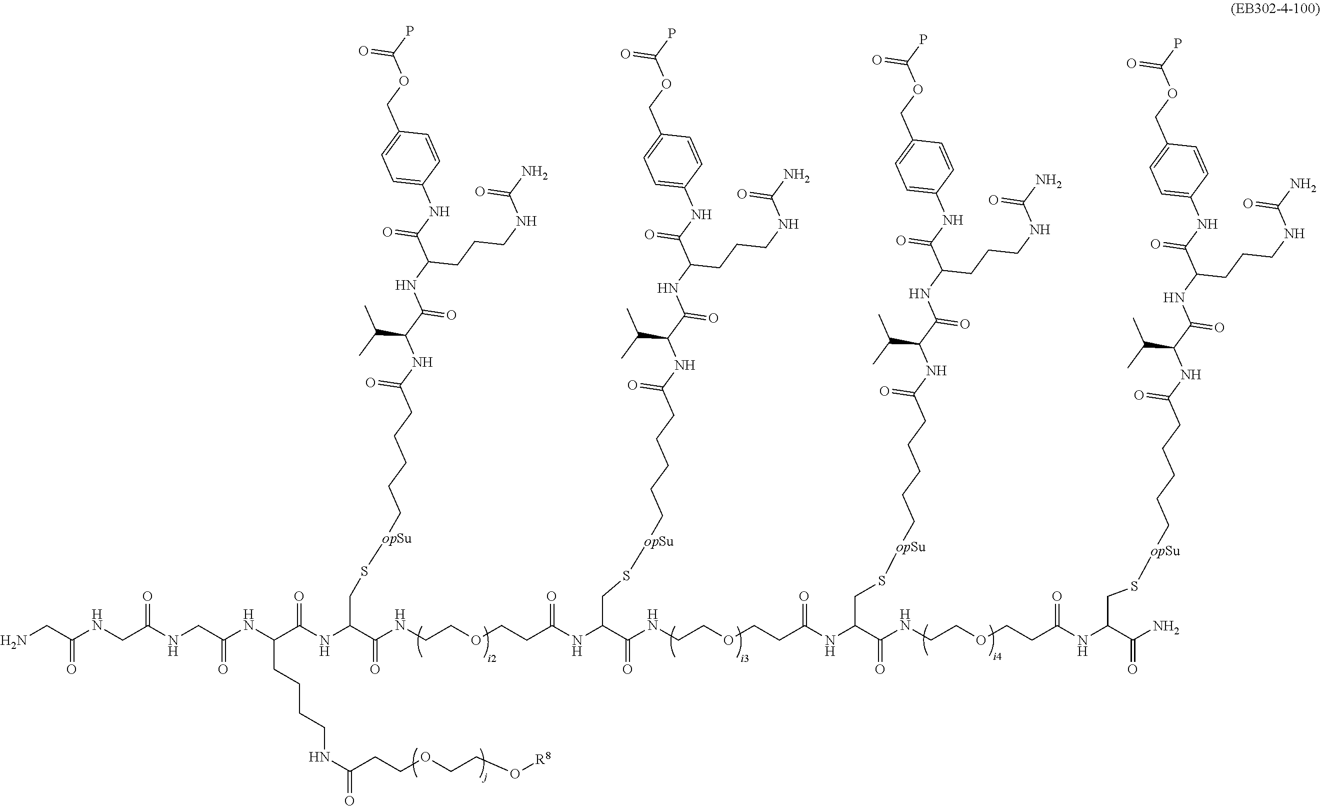

In an embodiment, i is 4. In an embodiment, EB301-2-100 is as follows (EB301-2-101):

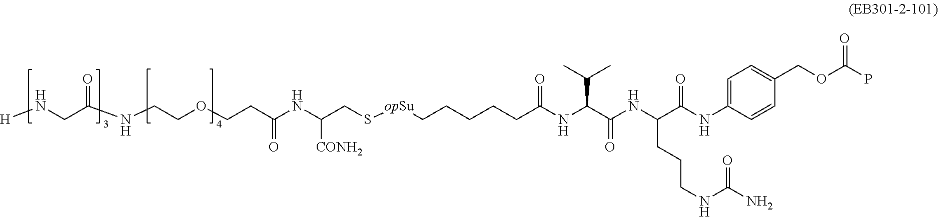

In an embodiment, i is 4, j is 8. In an embodiment, EB302-2-100 is as follows (EB302-2-101):

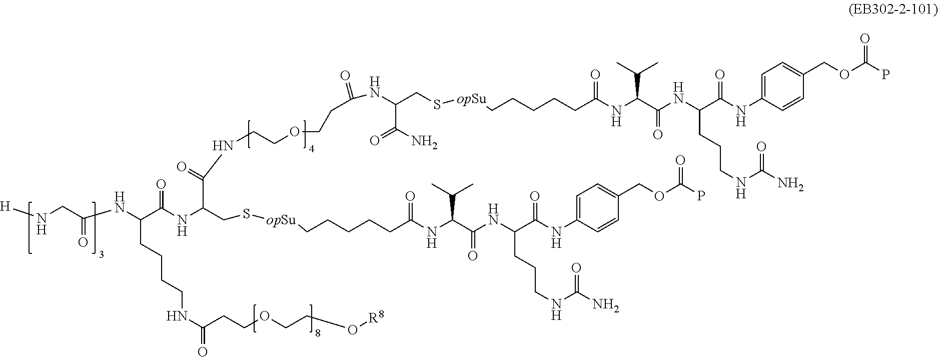

In an embodiment, i is 4, j is 12. In an embodiment, EB302-2-100 is as follows (EB302-2-104):

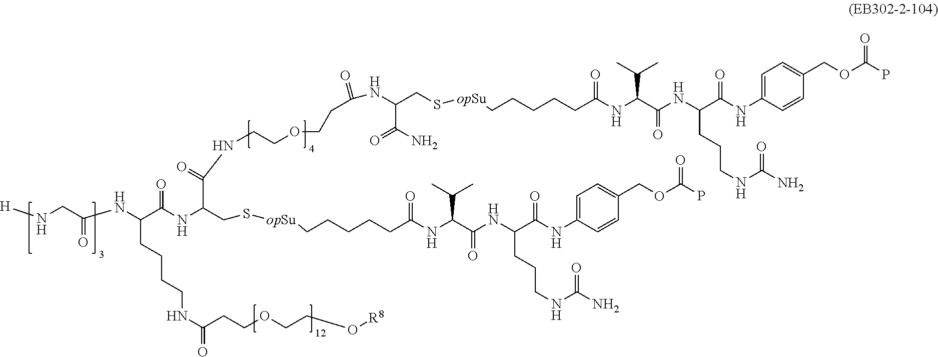

In one embodiment, the payload is a cytotoxin. In one embodiment, the compound of formula (II) (numbered as EBx) is as shown in the following Table 1.

TABLE 1
Specific linker-payload compounds

| Compound of | | Values of i and j | |
|---|---|---|---|
| formula (II) | Formula | (when applicable)* | Payload |
| EB301-1-1-1 | EB301-1-1 | — | formula (ii) |
| EB301-2-1-1 | EB301-2-0 | i is 4 | formula (ii) |
| EB301-3-1-1 | EB301-3-0 | i1 is 4, i2 is 4 | formula (ii) |
| EB301-4-1-1 | EB301-4-0 | i1, i2, i3 are 4, | formula (ii) |
| | | respectively | |
| EB301-5-1-1 | EB301-5-0 | i1, i2, i3 and i4 are 4, | formula (ii) |
| | | respectively | |
| EB302-1-1-1 | EB302-1-0 | j is 8 | formula (ii) |
| EB302-1-4-1 | EB302-1-0 | j is 12 | formula (ii) |
| EB302-2-1-1 | EB302-2-0 | i is 4, j is 8 | formula (ii) |
| EB302-2-4-1 | EB302-2-0 | i is 4, j is 12 | formula (ii) |
| EB302-3-1-1 | EB302-3-0 | i1 is 4, i2 is 4, j is 8 | formula (ii) |
| EB302-3-4-1 | EB302-3-0 | i1 is 4, i2 is 4, j is 12 | formula (ii) |
| EB302-4-1-1 | EB302-4-0 | i1, i2, i3 are 4, | formula (ii) |
| | | respectively, j is 8 | |
| EB302-4-4-1 | EB302-4-0 | i1, i2, i3 are 4, | formula (ii) |
| | | respectively, j is 12 | |
| EB301-1-101-1 | EB301-1-101 | — | formula (ii) |
| EB301-2-101-1 | EB301-2-100 | i is 4 | formula (ii) |
| EB301-3-101-1 | EB301-3-100 | i1 is 4, i2 is 4 | formula (ii) |
| EB301-4-101-1 | EB301-4-100 | i1, i2, i3 are 4, | formula (ii) |
| | | respectively | |
| EB301-5-101-1 | EB301-5-100 | i1, i2, i3 and i4 are 4, | formula (ii) |
| | | respectively | |
| EB302-1-101-1 | EB302-1-100 | j is 8 | formula (ii) |
| EB302-1-104-1 | EB302-1-100 | j is 12 | formula (ii) |
| EB302-2-101-1 | EB302-2-100 | i is 4, j is 8 | formula (ii) |
| EB302-2-104-1 | EB302-2-100 | i is 4, j is 12 | formula (ii) |
| EB302-3-101-1 | EB302-3-100 | i1 is 4, i2 is 4, j is 8 | formula (ii) |
| EB302-3-104-1 | EB302-3-100 | i1 is 4, i2 is 4, j is 12 | formula (ii) |
| EB302-4-101-1 | EB302-4-100 | i1, i2, i3 are 4, | formula (ii) |
| | | respectively, j is 8 | |
| EB302-4-104-1 | EB302-4-100 | i1, i2, i3 are 4, | formula (ii) |
| | | respectively, j is 12 | |

*For all the formula (II) compounds listed, n is 3.

### Conjugates and Preparation Thereof

Furthermore, the formula (II) compound which has the moiety comprising ligase recognition sequence can be conjugated with other molecules comprising a ligase recognition sequence, and can be thereby used in for example, the preparation of a targeting molecule-drug conjugate, such as an antibody-drug conjugate. Accordingly, in yet another aspect, provided is a conjugate which comprises a compound of formula (II) and a targeting molecule.

In yet another aspect, provided is a conjugate having the structure of formula (III):

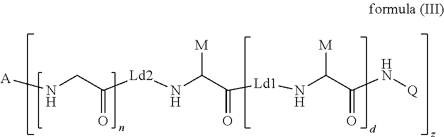

wherein,
Q is hydrogen or LKb-P;
M is hydrogen or LKa-LKb-P;
provided that Q and M are not simultaneously hydrogen;
A is a targeting molecule which is linked to the Gₙ moiety of the compound of formula (II); G is glycine;
z is an integer of 1 to 20;
P, n, d, Ld1 and Ld2 are as defined in formula (II).

In one embodiment, in the compound of formula (III), P is eribulin.

In a preferred embodiment, P has the structure of formula (ii)

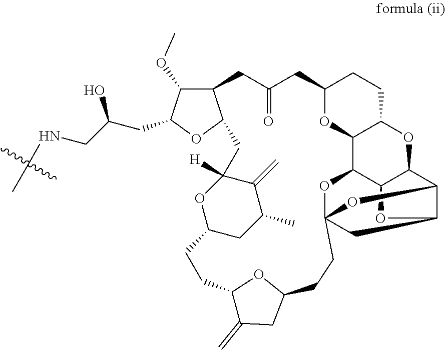 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

wherein the wavy bond shows the connection site for connection to the L¹ moiety.

In one embodiment, the targeting molecule is an antibody or an antigen binding fragment thereof. In one embodiment, the antibody is anti-CD19 antibody, anti-CD20 antibody, anti-CD22 antibody, anti-CD25 antibody, anti-CD30/TNFRSF8 antibody, anti-CD33 antibody, anti-CD37 antibody, anti-CD44v6 antibody, anti-CD56 antibody, anti-CD70 antibody, anti-CD71 antibody, anti-CD74 antibody, anti-CD79b antibody, anti-CD117/KITk antibody, anti-CD123 antibody, anti-CD138 antibody, anti-CD142 antibody, anti-CD174 antibody, anti-CD227/MUC1 antibody, anti-CD352 antibody, anti-CLDN18.2 antibody, anti-DLL3 antibody, anti-ErbB2/HER2 antibody, anti-CN33 antibody, anti-GPNMB antibody, anti-ENPP3 antibody, anti-Nectin-4 antibody, anti-EGFRvIII antibody, anti-SLC44A4 AGS-5 antibody, anti-CEACAM5 antibody, anti-PSMA antibody, anti-TIM1 antibody, anti-LY6E antibody, anti-LIV1 antibody, anti-Nectin4 antibody, anti-SLITRK6 antibody, anti-HGFR/cMet antibody, anti-SLAMF7/CS1 antibody, anti-EGFR antibody, anti-BCMA antibody, anti-AXL antibody, anti-NaPi2B antibody, anti-GCC antibody, anti-STEAP1 antibody, anti-MUC16 antibody, anti-Mesothelin antibody, anti-ETBR antibody, anti-EphA2 antibody, anti-5T4 antibody, anti-FOLR1 antibody, anti-LAMP1 antibody, anti-Cadherin 6 antibody, anti-FGFR2 antibody, anti-FGFR3 antibody, anti-CA6 antibody, anti-CanAg antibody, anti-integrin αV antibody, anti-TDGF1 antibody, anti-Ephrin A4 antibody, anti-TROP2 antibody, anti-PTK7 antibody, anti-NOTCH3 antibody, anti-C4.4A antibody, anti-FLT3 antibody, anti-B7H3/4 antibody, anti-Tissue Factor antibody, anti-ROR1/2 antibody, preferably, anti-HER2 antibody, anti-FGFR3 antibody, anti-Trop2 antibody, anti-HER3 antibody or anti-FRα antibody.

In one embodiment, the targeting molecule is anti-HER2 antibody. In one embodiment, the targeting molecule is an anti-human HER2 antibody or antigen binding fragment thereof. Examples of anti-human HER2 antibodies include but are not limited to Pertuzumab and Trastuzumab. Pertuzumab binds to the second extracellular domain (ECD2) of HER2 and is approved for the treatment of HER2-positive breast cancer. Trastuzumab binds to the fourth extracellular domain (ECD4) of HER2 and is approved for the treatment of Her2-positive breast cancer and gastric cancer.

In one embodiment, the antibody is an anti-HER2 antibody or antigen-binding fragment thereof, comprising a light chain variable region (V_{L}) and a heavy chain variable region (V_{H}), wherein the V_{H} comprises HCDR1 comprising the amino acid sequence of SEQ ID NO: 1. HCDR2 comprising the amino acid sequence of SEQ ID NO: 2, HCDR3 comprising the amino acid sequence of SEQ ID NO: 3, and wherein the V_{L} comprises LCDR1 comprising the amino acid sequence of SEQ ID NO: 4, LCDR2 comprising the amino acid sequence of SEQ ID NO: 5, LCDR3 comprising the amino acid sequence of SEQ ID NO: 6. In one embodiment, the CDR regions are defined according to KABAT. In one embodiment, the antibody is an anti-HER2 antibody or antigen-binding fragment thereof, comprising a light chain variable region (V_{L}) and a heavy chain variable region (V_{H}), wherein the V_{L} has an amino acid sequence having at least about 85%, at least about 90% or at least about 95% sequence identity with SEQ ID NO: 7, and the V_{H} has an amino acid sequence having at least about 85%, at least about 90% or at least about 95% sequence identity with SEQ ID NO: 8. In a preferred embodiment, the anti-HER2 antibody or antigen-binding fragment thereof comprises a light chain variable region (V_{L}) and a heavy chain variable region (V_{H}), wherein the V_{L} has the amino acid sequence of SEQ ID NO: 7, and the V_{H} has the amino acid sequence of SEQ ID NO: 8. In a particular embodiment, the anti-HER2 antibody comprises a light chain and a heavy chain, wherein the light chain has an amino acid sequence having at least about 85%, at least about 90% or at least about 95% sequence identity with the amino acid sequence of amino acids 1 to 214 of SEQ ID NO: 9, and the heavy chain has an amino acid sequence having at least about 85%, at least about 90% or at least about 95% sequence identity with SEQ ID NO: 10. In a preferred embodiment, the anti-human HER2 antibody is one or more selected from engineered anti-HER2 antibodies based on Pertuzumab. In one embodiment, the antibody is a modified Pertuzumab, preferably Ab0036 (having light chain SEQ ID NO: 9, heavy chain: SEQ ID NO: 10). The sequence of Ab0036 is based on the amino acid sequence of Pertuzumab, and GALPETGG was introduced at the C-terminal of the light chain, wherein LPETGG is the recognition sequence of the ligase donor substrate, and GA is a spacer sequence.

In one embodiment, the targeting molecule is one or more selected from anti-human FGFR3 antibodies or antigen-binding fragment thereof. In one embodiment, the anti-FGFR3 antibody or antigen-binding fragment thereof comprises a light chain variable region (V_{L}) and a heavy chain variable region (V_{H}), wherein the V_{H} comprises HCDR1 comprising the amino acid sequence of SEQ ID NO: 11, HCDR2 comprising the amino acid sequence of SEQ ID NO: 12, HCDR3 comprising the amino acid sequence of SEQ ID NO: 13, and wherein the V_{L} comprises LCDR1 comprising the amino acid sequence of SEQ ID NO: 14, LCDR2 comprising the amino acid sequence of SEQ ID NO: 15, LCDR3 comprising the amino acid sequence of SEQ ID NO: 16. In one embodiment, the CDR regions are defined according to KABAT. In one embodiment, the anti-FGFR3 antibody or antigen-binding fragment thereof comprises a light chain variable region (V_{L}) and a heavy chain variable region (V_{H}), wherein the V_{L} has an amino acid sequence having at least about 85%, at least about 90% or at least about 95% sequence identity with SEQ ID NO: 17, and the V_{H} has an amino acid sequence having at least about 85%, at least about 90% or at least about 95% sequence identity with SEQ ID NO: 18. In a preferred embodiment, the anti-FGFR3 antibody or antigen-binding fragment thereof comprises a light chain variable region (V_{L}) and a heavy chain variable region (V_{H}), wherein the V_{L} has the amino acid sequence of SEQ ID NO: 17, and the V_{H} has the amino acid sequence of SEQ ID NO: 18. In a particular embodiment, the anti-FGFR3 antibody comprises a light chain and a heavy chain, wherein the light chain has an amino acid sequence having at least about 85%, at least about 90% or at least about 95% sequence identity with the amino acid sequence of amino acids 1 to 214 of SEQ ID NO: 19, and the heavy chain has an amino acid sequence having at least about 85%, at least about 90% or at least about 95% sequence identity with SEQ ID NO: 20. In one embodiment, the antibody is Ab0206 (having light chain SEQ ID NO: 19, heavy chain: SEQ ID NO: 20). Ab0206 includes GALPETGG at the C-terminal of the light chain of Ab0206, wherein LPETGG is the recognition sequence of the ligase donor substrate, and GA is a spacer sequence.

In one embodiment, the antibody is an anti-HER2 antibody or antigen-binding fragment thereof, comprising a light chain variable region (V_{L}) and a heavy chain variable region (V_{H}), wherein the V_{H} comprises HCDR1 comprising the amino acid sequence of SEQ ID NO: 21, HCDR2 comprising the amino acid sequence of SEQ ID NO: 22, HCDR3 comprising the amino acid sequence of SEQ ID NO: 23, and wherein the V_{L} comprises LCDR1 comprising the amino acid sequence of SEQ ID NO: 24, LCDR2 comprising the amino acid sequence of SEQ ID NO: 25, LCDR3 comprising the amino acid sequence of SEQ ID NO: 26. In one embodiment, the CDR regions are defined according to KABAT. In one embodiment, the antibody is an anti-HER2 antibody or antigen-binding fragment thereof, comprising a light chain variable region (V_{L}) and a heavy chain variable region (V_{H}), wherein the V_{L} has an amino acid sequence having at least about 85%, at least about 90% or at least about 95% sequence identity with SEQ ID NO: 27, and the V_{H} has an amino acid sequence having at least about 85%, at least about 90% or at least about 95% sequence identity with SEQ ID NO: 28. In a preferred embodiment, the anti-HER2 antibody or antigen-binding fragment thereof comprises a light chain variable region (V_{L}) and a heavy chain variable region (V_{H}), wherein the V_{L} has the amino acid sequence of SEQ ID NO: 27, and the V_{H} has the amino acid sequence of SEQ ID NO: 28. In a particular embodiment, the anti-HER2 antibody comprises a light chain and a heavy chain, wherein the light chain has an amino acid sequence having at least about 85%, at least about 90% or at least about 95% sequence identity with the amino acid sequence of amino acids 1 to 214 of SEQ ID NO: 29, and the heavy chain has an amino acid sequence having at least about 85%, at least about 90% or at least about 95% sequence identity with SEQ ID NO: 30. In a preferred embodiment, the anti-human HER2 antibody is one or more selected from engineered anti-HER2 antibodies based on Trastuzumab. In one embodiment, the antibody is a modified Trastuzumab, preferably Ab0001 (having light chain SEQ ID NO: 29, heavy chain: SEQ ID NO: 30). The sequence of Ab0001 is based on the amino acid sequence of Trastuzumab, and GALPETGG was introduced at the C-terminal of the light chain, wherein LPETGG is the recognition sequence of the ligase donor substrate, and GA is a spacer sequence. The specific sequences can be found in the attached Sequence Listing.

In a preferred embodiment, the anti-human HER2 antibody or anti-human FGFR3 antibody is a recombinant antibody selected from monoclonal antibody, chimeric antibody, humanized antibody, antibody fragment, and antibody mimic. In one embodiment, the antibody mimic is selected from scFv, minibody, diabody, nanobody. For the conjugation with the compound of formula (II), the targeting molecule of the present disclosure may comprise a modified moiety to connect with Gₙ moiety in the compound of formula (II). The introduction position of such modified moiety is not limited, for example, when the targeting molecule is an antibody, its introduction position can be, but not limited to, located at the C-terminal or the N-terminal of the heavy chain or light chain of the antibody.

In an alternative embodiment, a modified moiety for the conjugation with Gₙ moiety in the compound of formula (II) can be introduced at a non-terminal position of the heavy chain or light chain of the antibody using, for example, chemical modification methods.

In one embodiment, the targeting molecule of the present disclosure is an antibody or antigen-binding fragment thereof, which may comprise terminal modification. A terminal modification refers to a modification at the C-terminal or N-terminal of the heavy chain or light chain of the antibody, which for example comprises a ligase recognition sequence. In another embodiment, the terminal modification may further comprise spacer Sp2 comprising 2-100 amino acids, wherein the antibody, Sp2 and the ligase recognition sequence are sequentially linked. In a preferred embodiment, Sp2 is a spacer sequence containing 2-20 amino acids. In a particular embodiment, Sp2 is a spacer sequence selected from GA, GGGGS, GGGGSGGGGS and GGGGSGGGGSGGGGS, especially GA.

In a preferred embodiment, the light chain of the antibody or antigen-binding fragment thereof includes 3 types: wild-type (LC); the C-terminus modified light chain (LCCT), which is modified by direct introduction of a ligase recognition sequence LPXTG and C-terminus modified light chain (LCCT_{L}), which is modified by introduction of short peptide spacers plus the ligase donor substrate recognition sequence LPXTG. The heavy chain of the antibody or antigen-binding fragment thereof includes 3 types: wild-type (HC); the C-terminus modified heavy chain (HCCT), which is modified by direct introduction of a ligase recognition sequence LPXTG; and C-terminus modified heavy chain (HCCT_{L}), which is modified by introduction of short peptide spacers plus the ligase donor substrate recognition sequence LPXTG. X can be any natural or non-natural single amino acid. When z in the compound of formula (III) is 1 or 2, the combination of the above heavy and light chains can form 8 preferred antibody molecules.

In one embodiment, the targeting molecule is an anti-human FGFR3 antibody, preferably Ab0206; or an anti-human HER2 antibody, preferably Ab0036 or Ab0001. In another embodiment, the targeting molecule is an anti-human FGFR3 antibody, preferably Ab0206; or the targeting molecule is Ab0036.

In one embodiment, in the compound of formula (III), the targeting molecule is an anti-human FGFR3 antibody, preferably Ab0206; or the targeting molecule is Ab0036. In another embodiment, the targeting molecule is Ab0001.

In one embodiment, the compound of formula (III) has the structure of formula (III-i)

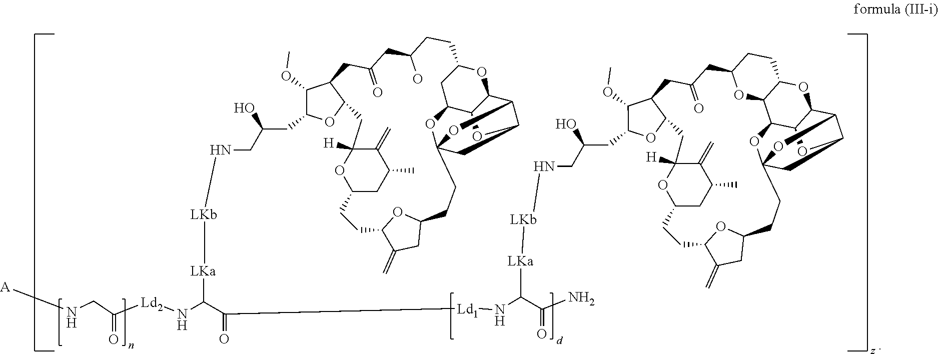

In one embodiment, in the compound of formula (III-i), the targeting molecule is an anti-human FGFR3 antibody, preferably Ab0206; or an anti-human HER2 antibody, preferably or Ab0001.

In one embodiment, P, n, d, Ld1, Ld2, LKa and LKb are as defined in formula (II).

As defined herein above, in one embodiment, the Gₙ moiety of the compound of formula (II) is a recognition sequence of a ligase acceptor or donor substrate, which facilitates enzyme-catalyzed coupling of compound of formula (II) with the targeting molecule under the catalysis of the ligase. The targeting molecule optionally modified and comprises the corresponding recognition sequence of a ligase acceptor or donor substrate.

It should be understood that, when the targeting molecule A conjugates with the Gₙ moiety of the compound of formula (II) under the catalysis of the ligase, the recognition sequence of the ligase acceptor substrate and the recognition sequence of the ligase donor substrate react with each other and form a resulting sequence.

In one embodiment, the targeting molecule A comprises LPXTGJ as the recognition sequence of the ligase donor substrate, wherein J is as defined above. When conjugates with Gₙ, which is the corresponding recognition sequence of the ligase acceptor substrate, the upstream peptide bond of the glycine in the LPXTGJ sequence is cleaved by Sortase A, and the resulting intermediate is linked to the free N-terminal of Gₙ to generate a new peptide bond. The resulting sequence is LPXTGₙ. The sequences Gₙ and LPXTGJ are as defined above.

Therefore, in one embodiment, the Gₙ moiety in the compound of formula (II) is the recognition sequence of the ligase acceptor substrate of a ligase; and the antibody is modified to comprises the recognition sequence of the ligase donor substrate in order to connect with the Gₙ moiety in the compound of formula (II);
preferably,
(a) the ligase is a Sortase selected from Sortase A, Sortase B, Sortase C, Sortase D and Sortase L. plantarum; preferably Sortase A from Staphylococcus aureus; and/or
(b) the recognition sequence of the ligase donor substrate is LPXTGJ, J is absent or is Gₘ, wherein G is glycine, m is an integer of 1 to 10; preferably, the recognition sequence of the ligase donor substrate is LPXTG or LPETGG; and/or
the connection of LPXTGJ with Gₙ results in LPXTGₙ.

In one embodiment, P is linked to the L¹ moiety of the compound of formula (II) and A is linked to the Gₙ moiety of the compound of formula (II) to form the compound of formula (III).

As defined above, in the A-Gₙ structure of the compound of formula (III), A optionally comprises the corresponding sequence resulting from the reaction of the recognition sequence of the ligase acceptor substrate with the recognition sequence of the ligase donor substrate.

The conjugates of the present disclosure can further comprise a payload. The payload is as described above.

### Specific Embodiments for the Conjugate

In one embodiment, Q is hydrogen, each LKa is

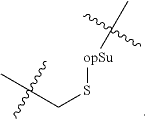

In one embodiment, formula (III) has the structure of formula (III-1):

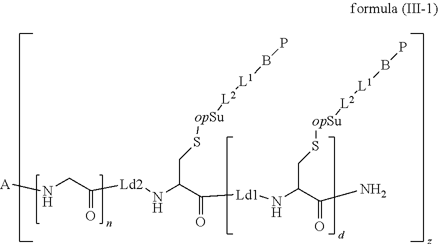

In one embodiment, Ld2 is a bond, d is 0. In one embodiment, the compound of formula (III-1) is as follows:

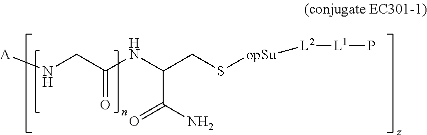

In one embodiment, d is 0, Ld2 is

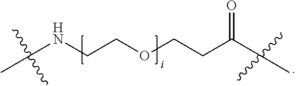

In one embodiment, the compound of formula (III-1) is as follows:

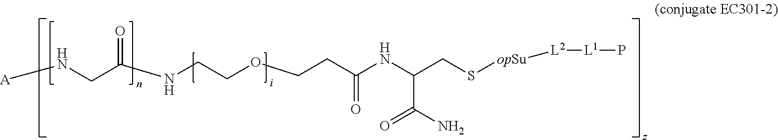

In one embodiment, d is 1, 2 or 3, Ld2 and each Ld1 are independently selected from

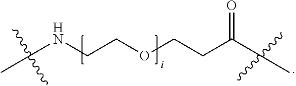

In one embodiment, the compound of formula (III-1) is as follows:

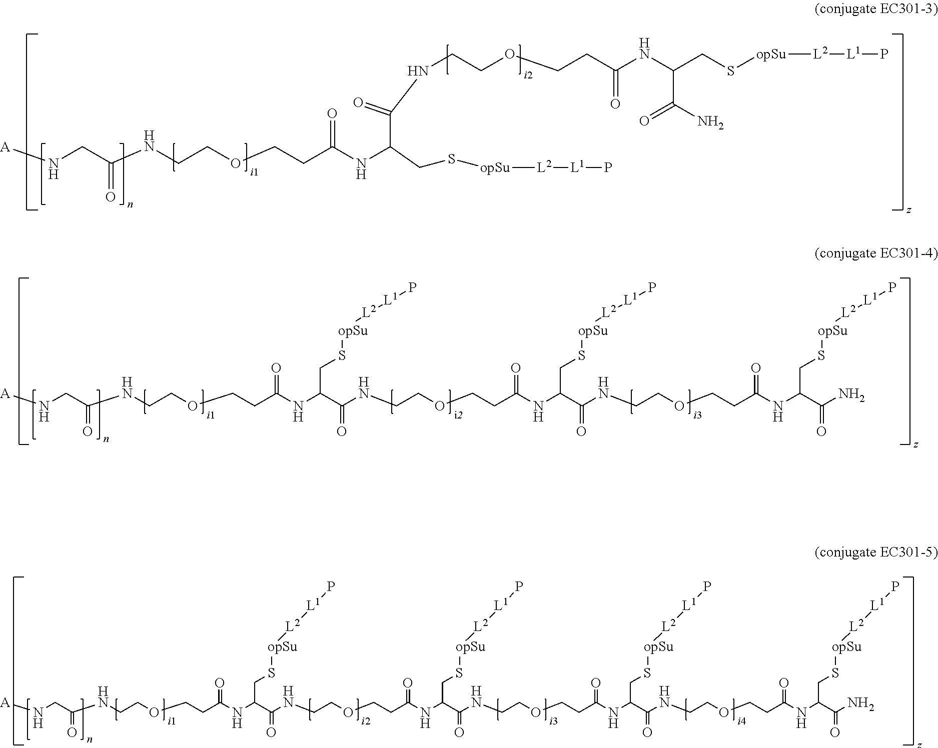

In one embodiment, Ld2 is

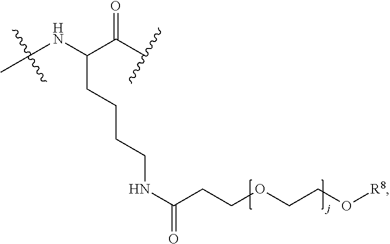

d is 0. In one embodiment, the compound of formula (III-1) is as follows:

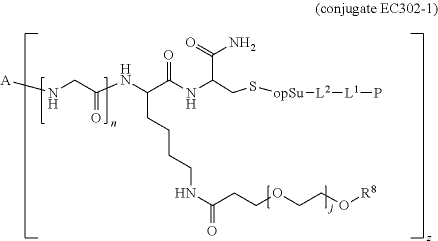

In one embodiment, d is 1, 2 or 3, Ld2 is

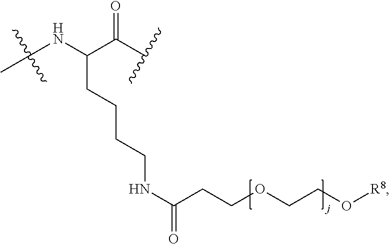

and each Ld1 is independently selected from

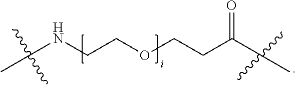

In one embodiment, the compound of formula (III-1) is as follows:

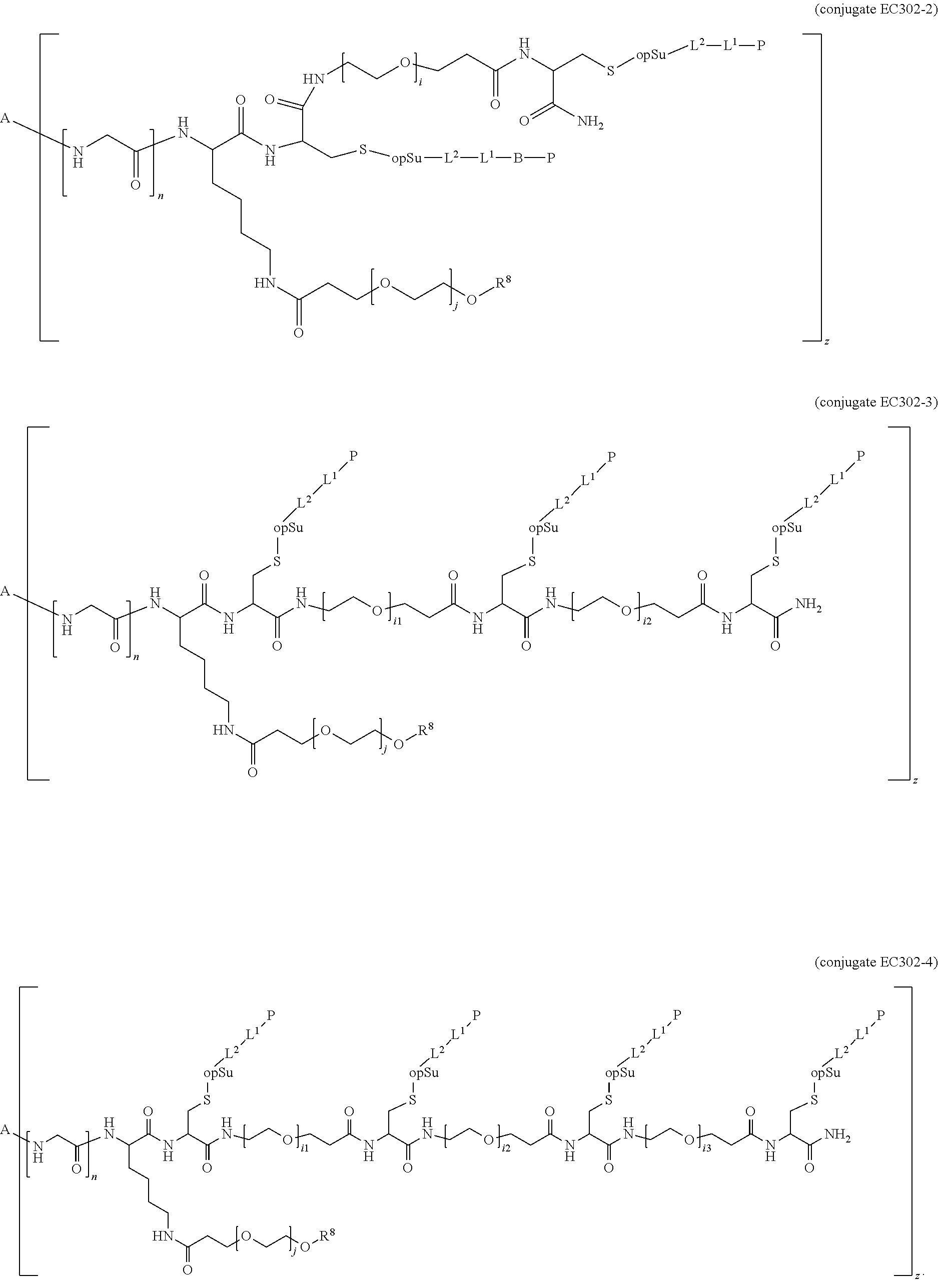

In an embodiment, z is 1 to 4. In an embodiment, z is 2 or 4. In an embodiment, z is 2. In an embodiment, in conjugate EC301-1, EC301-2, EC302-1, z is 2 or 4. In an embodiment, in conjugate EC301-3, EC301-4, EC301-5, EC302-2, EC302-3 and EC302-4, z is 2.

In one embodiment, n is 3, L² is —(CH₂)ₚ—(CH₂)₂(CO)—, p is 3, L¹ is GGFG. In one embodiment, conjugate EC301-1 has the structure of EC301-1-1:

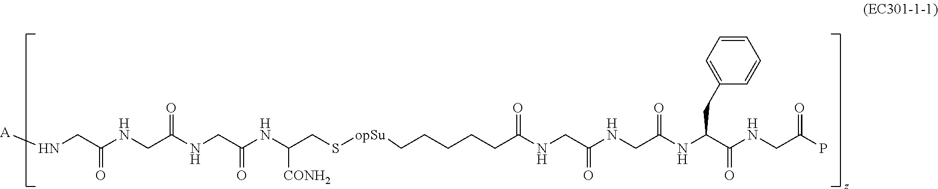

In one embodiment, conjugate EC301-2 has the structure of EC301-2-0:

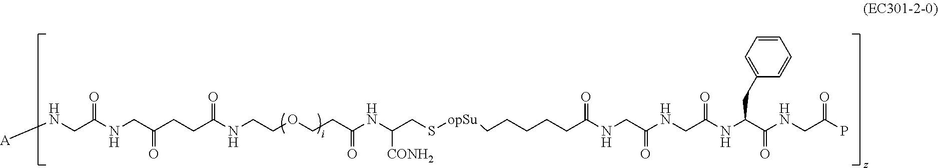

In one embodiment, conjugate EC301-3 has the structure of EC301-3-0:

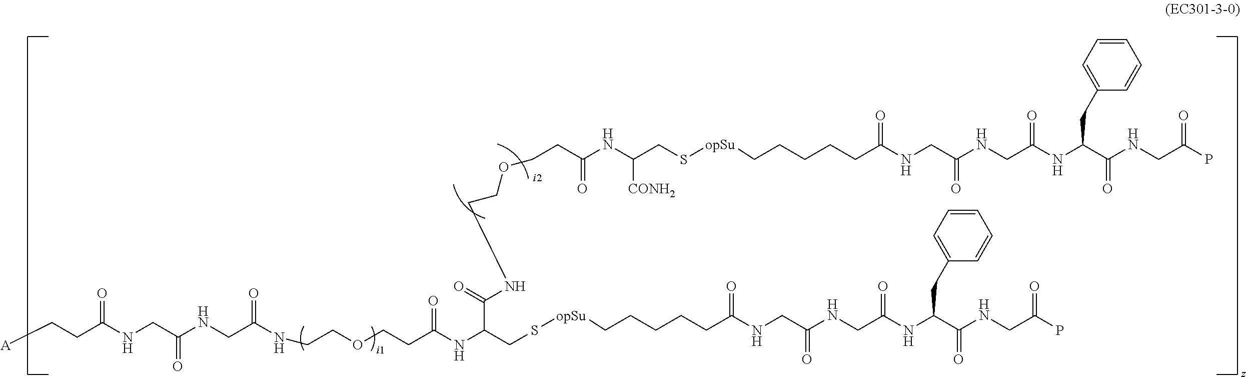

In one embodiment, conjugate EC301-4 has the structure of EC301-4-0:

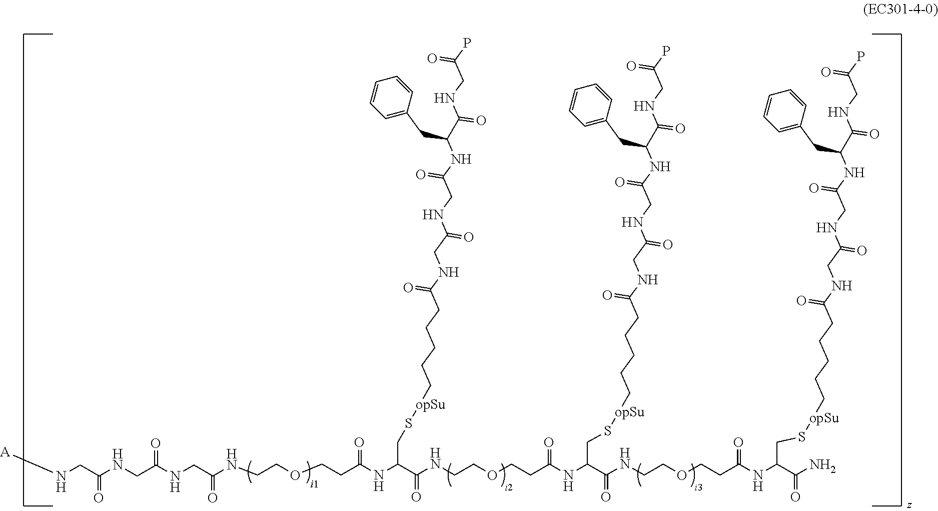

In one embodiment, conjugate EC301-5 has the structure of EC301-5-0:

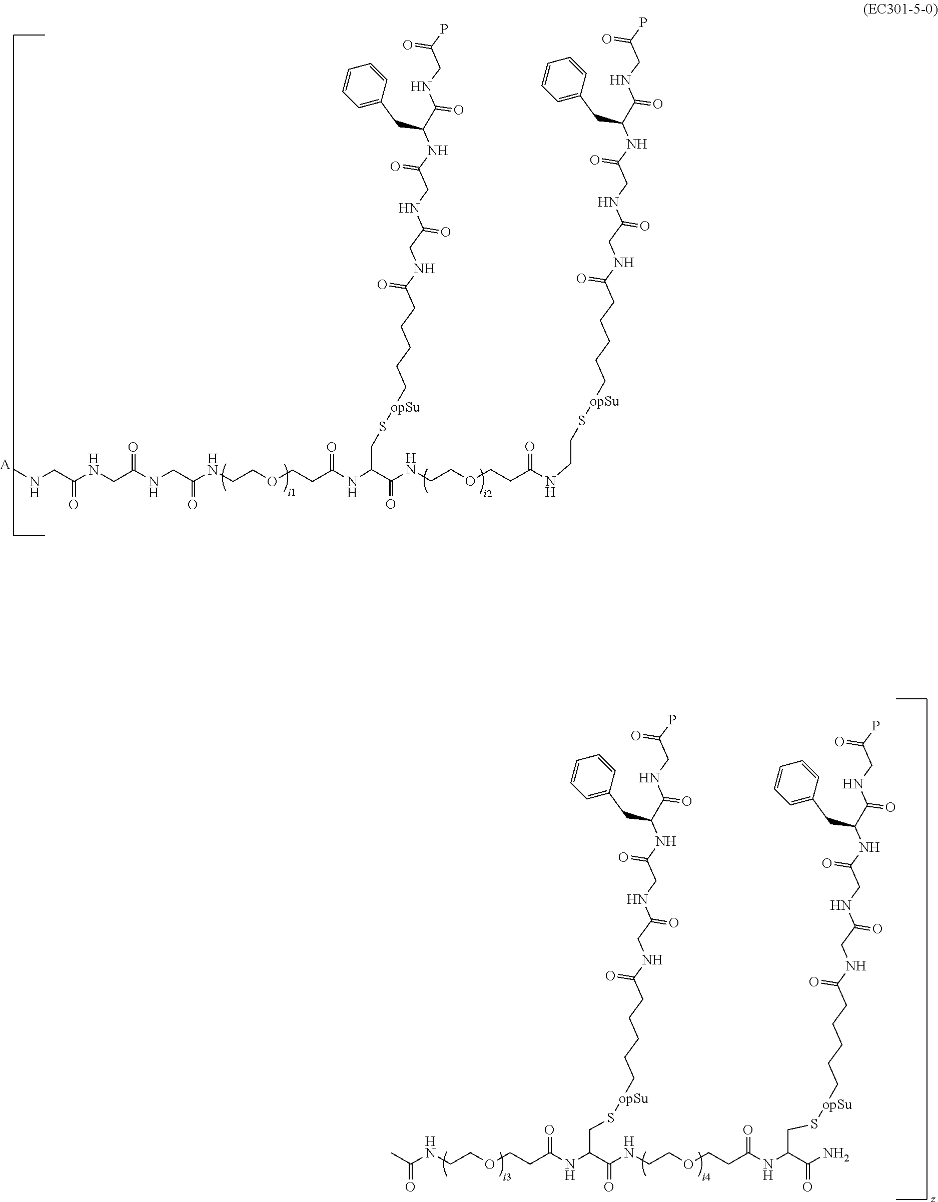

In one embodiment, conjugate EC302-1 has the structure of EC302-1-0:

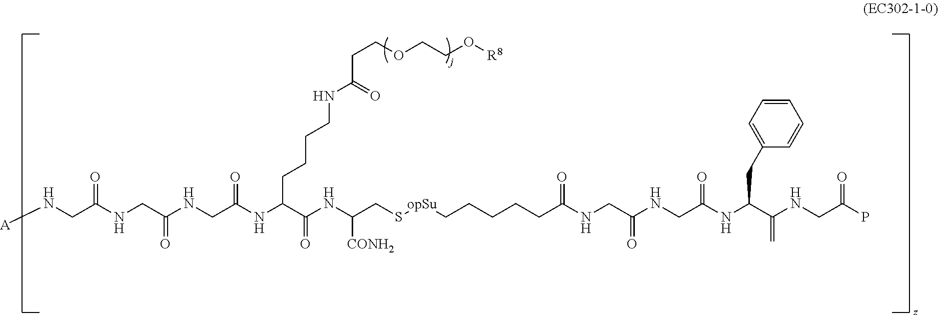

In one embodiment, conjugate EC302-2 has the structure of EC302-2-0:

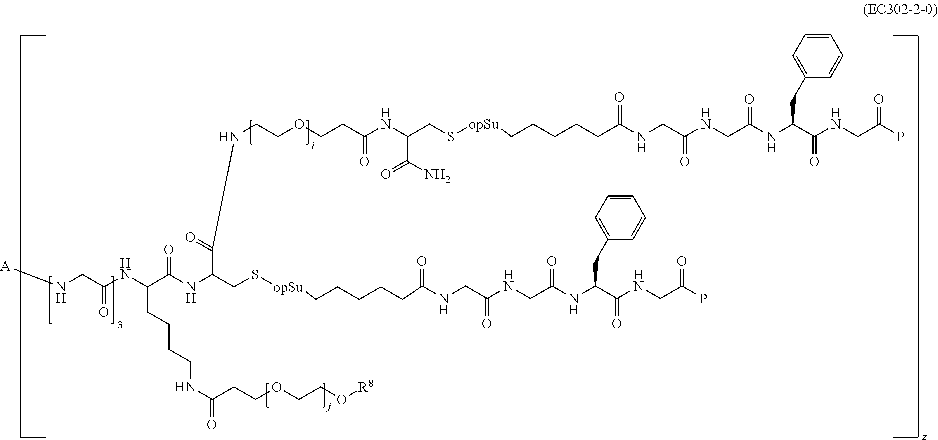

In one embodiment, conjugate EC302-3 has the structure of EC302-3-0:

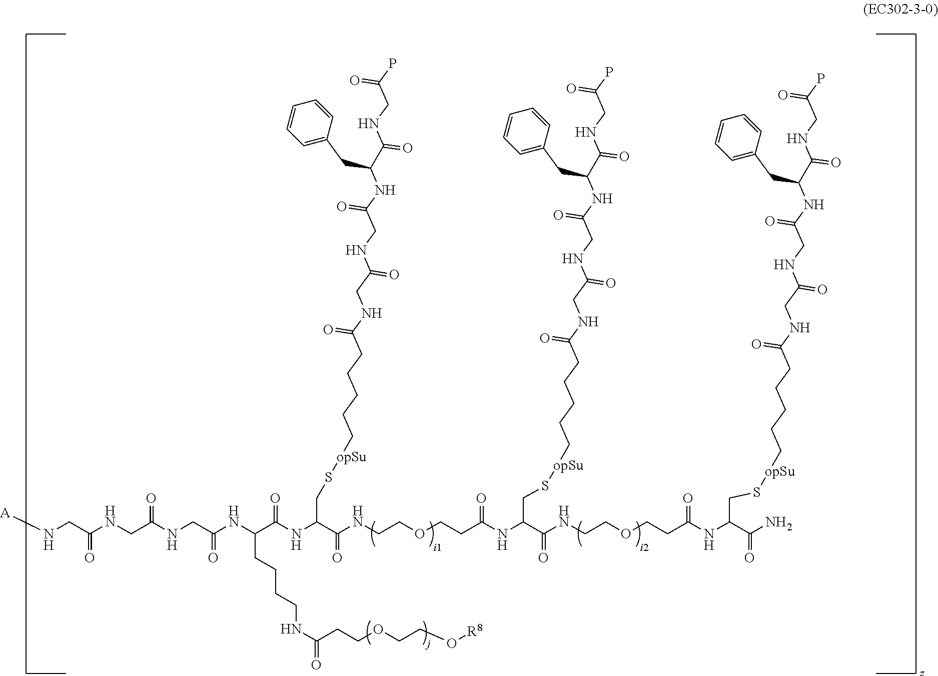

In one embodiment, conjugate EC302-4 has the structure of EC302-4-0:

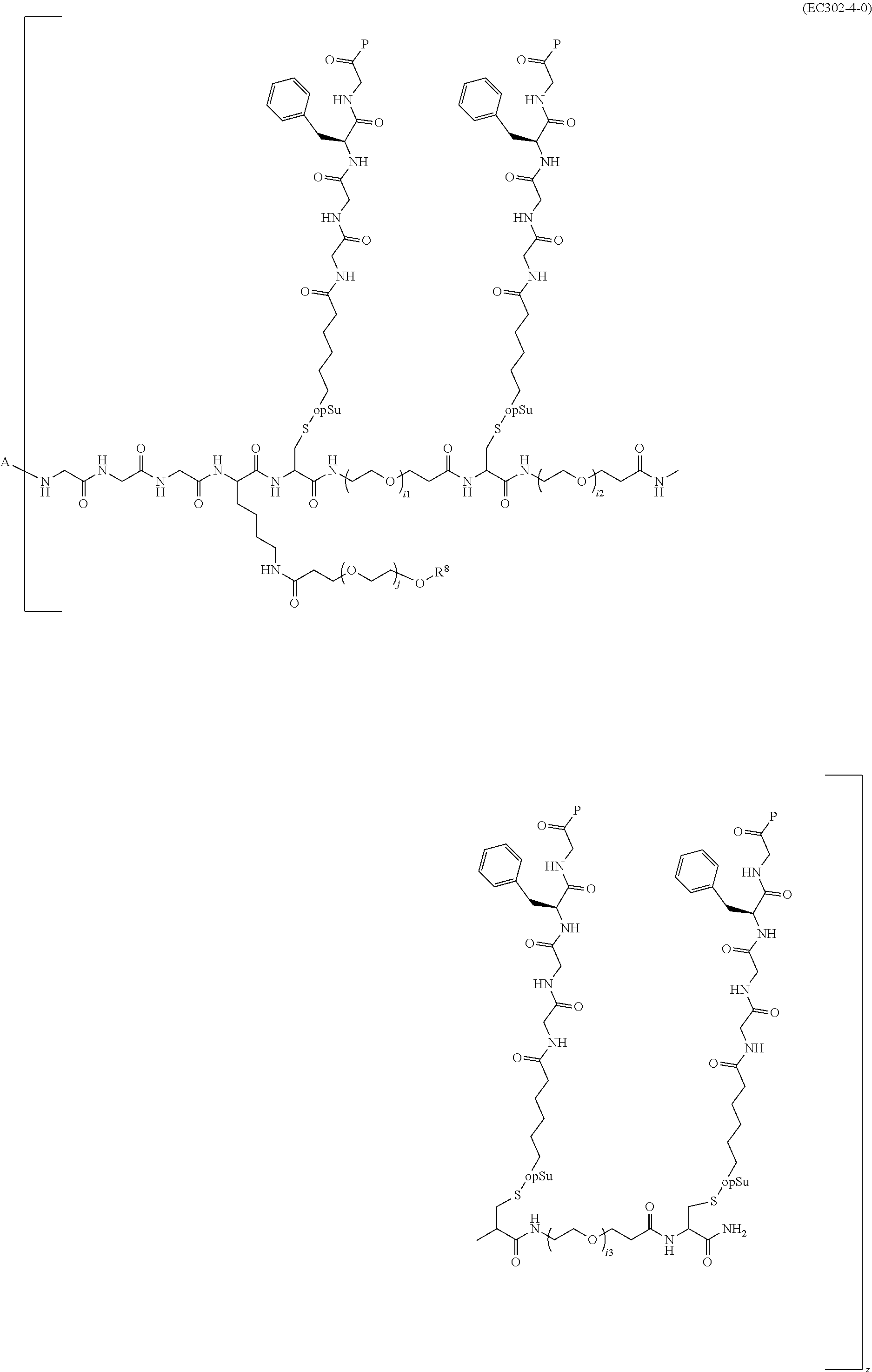

In one embodiment, conjugate EC301-1-1 has the structure of EC301-1-1-1:

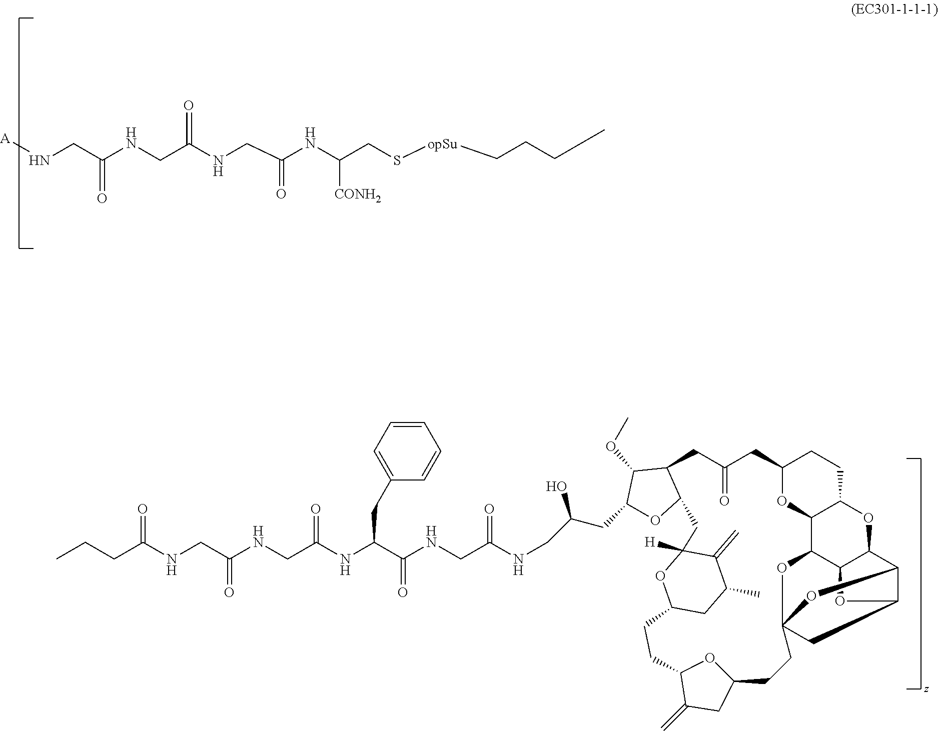

In one embodiment, conjugate EC301-2-0 has the structure of EC301-2-0-1:

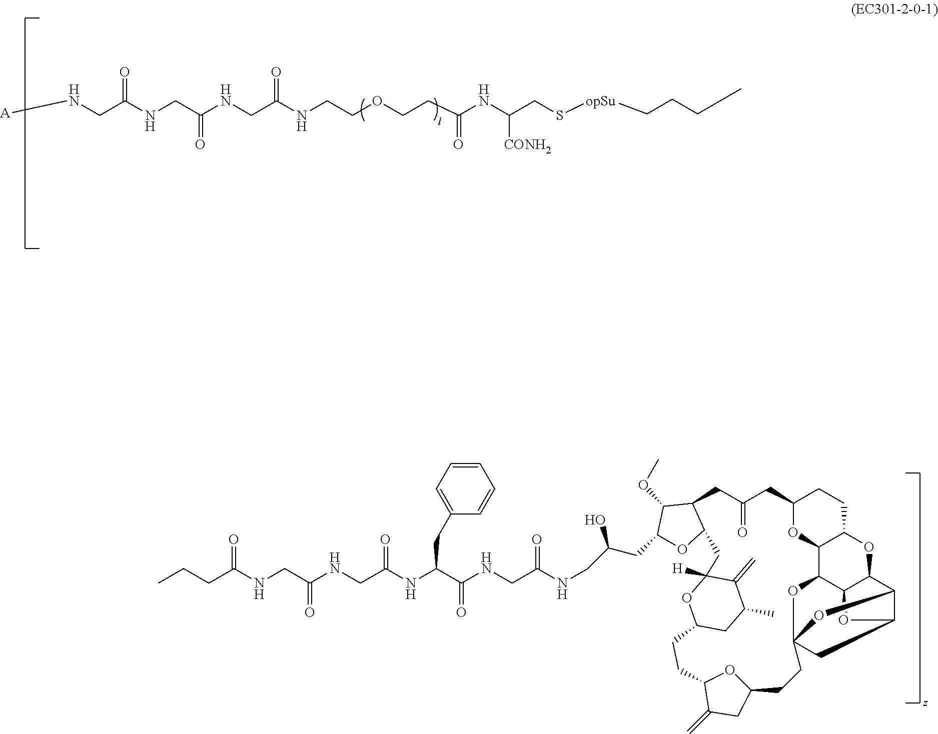

In one embodiment, conjugate EC301-3-0 has the structure of EC301-3-0-1:

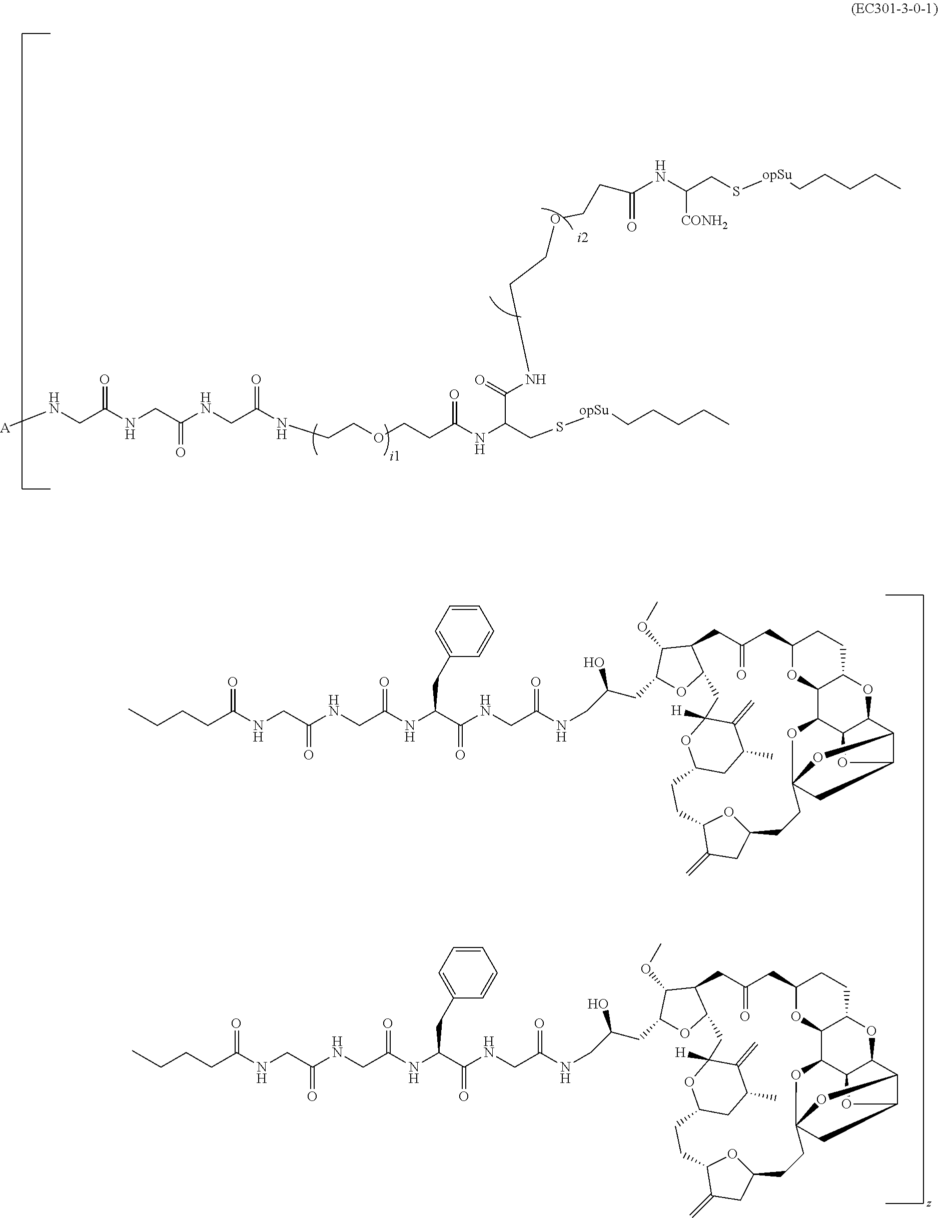

In one embodiment, conjugate EC301-4-0 has the structure of EC301-4-0-1:

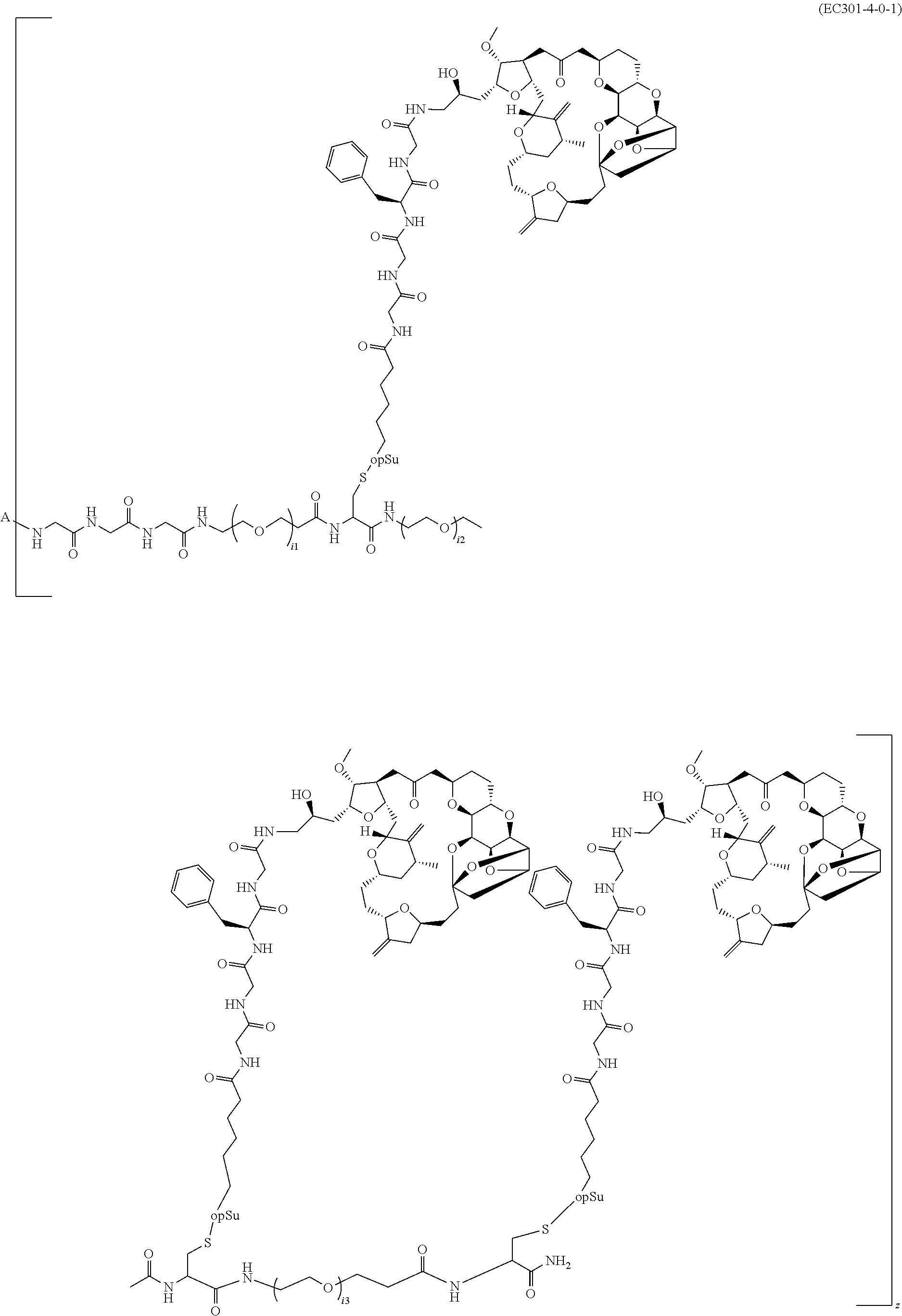

In one embodiment, conjugate EC301-5-0 has the structure of EC301-5-0-1:

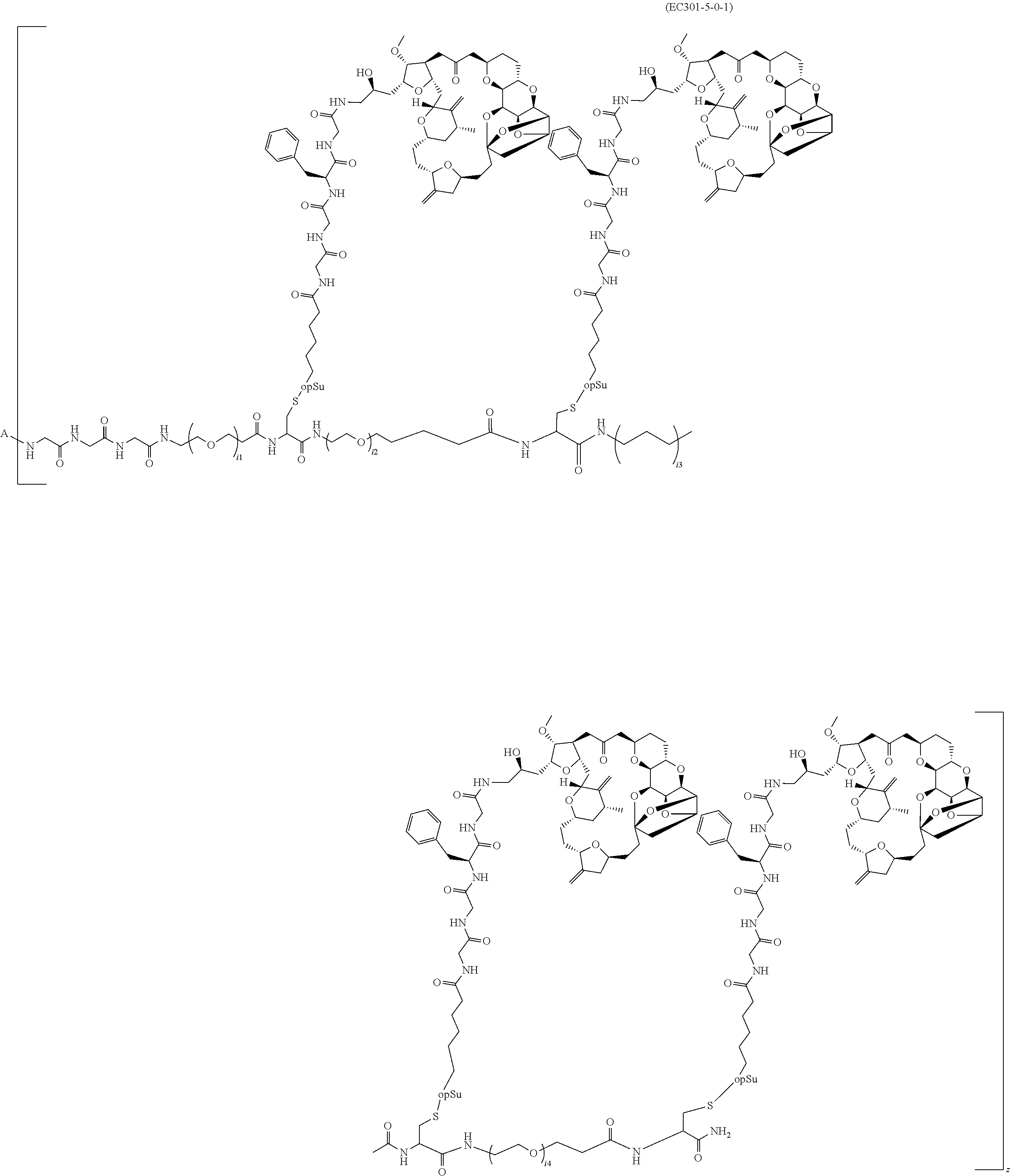

In one embodiment, conjugate EC302-1-0 has the structure of EC302-1-0-1:

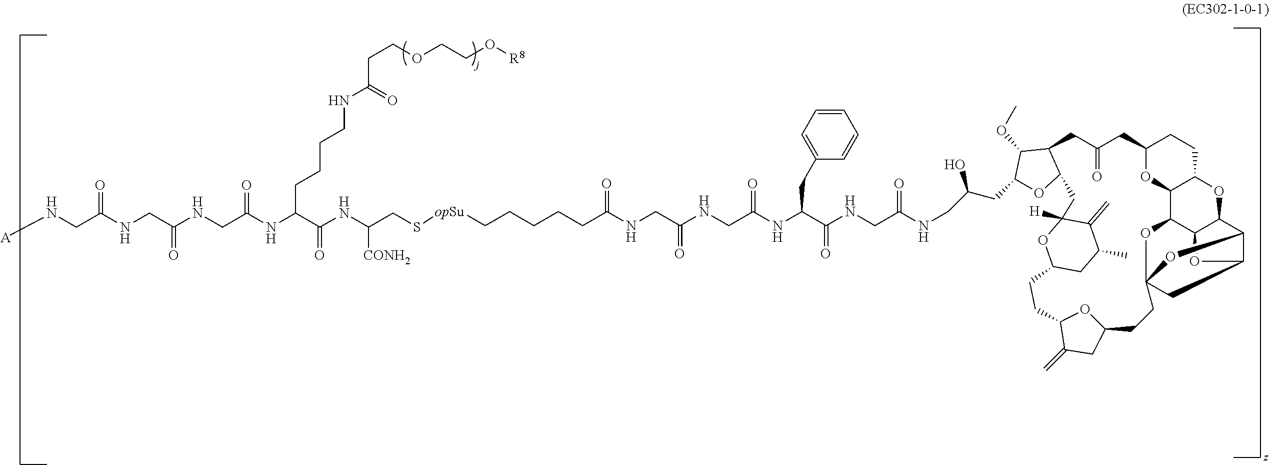

In one embodiment, conjugate EC302-2-0 has the structure of EC302-2-0-1:

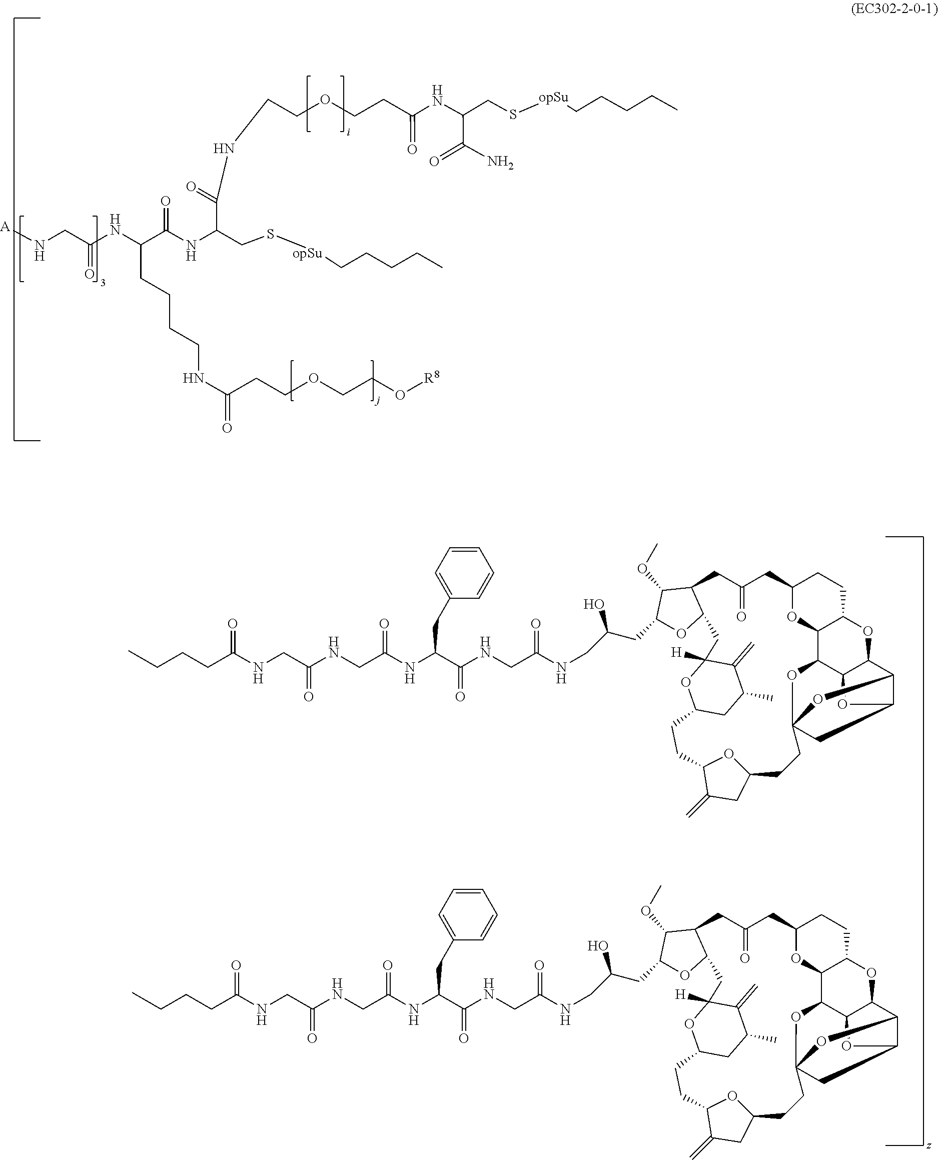

In one embodiment, conjugate EC302-3-0 bas the structure of EC302-3-0-1:

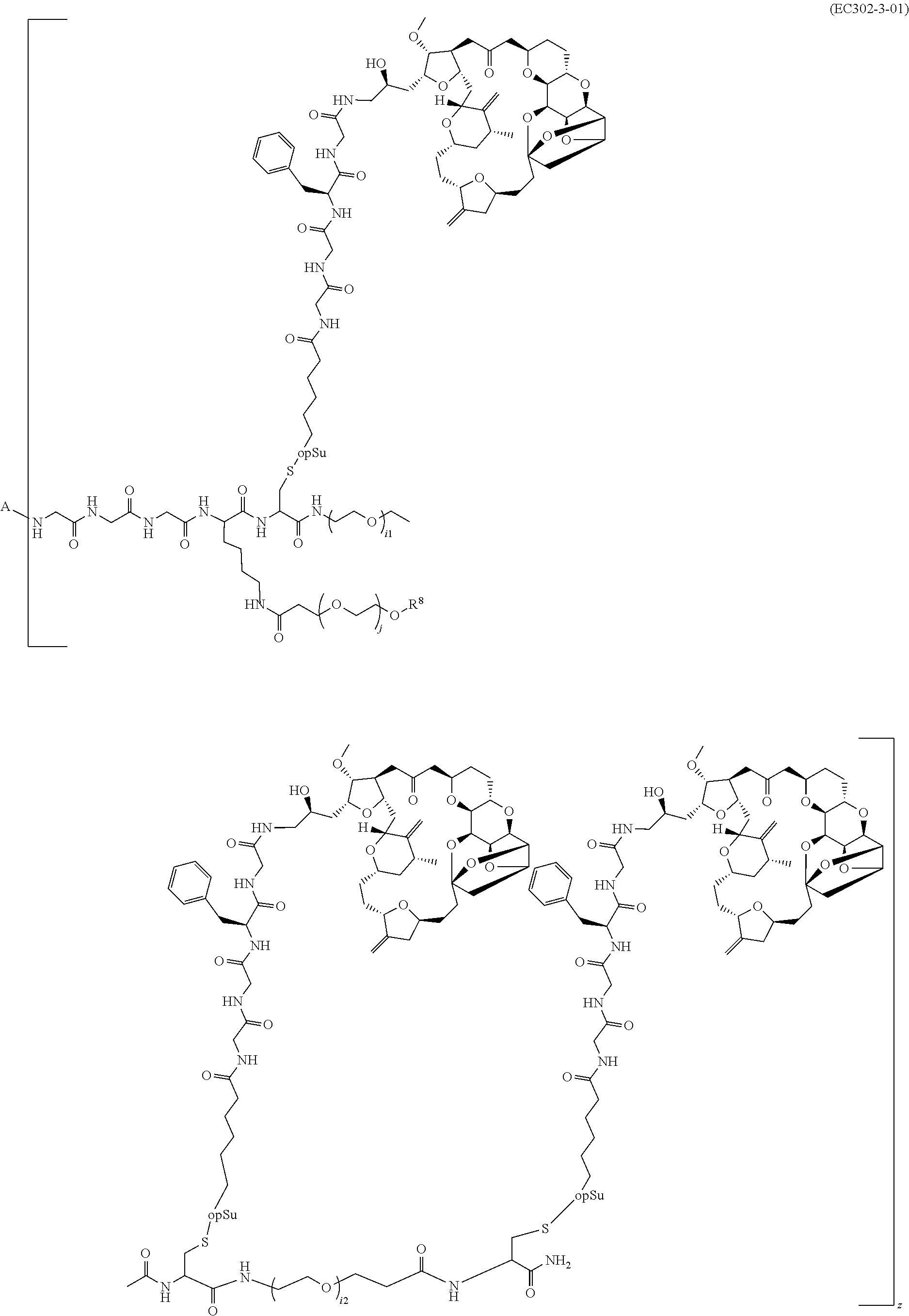

In one embodiment, conjugate EC302-4-0 bas the structure of EC302-4-0-1:

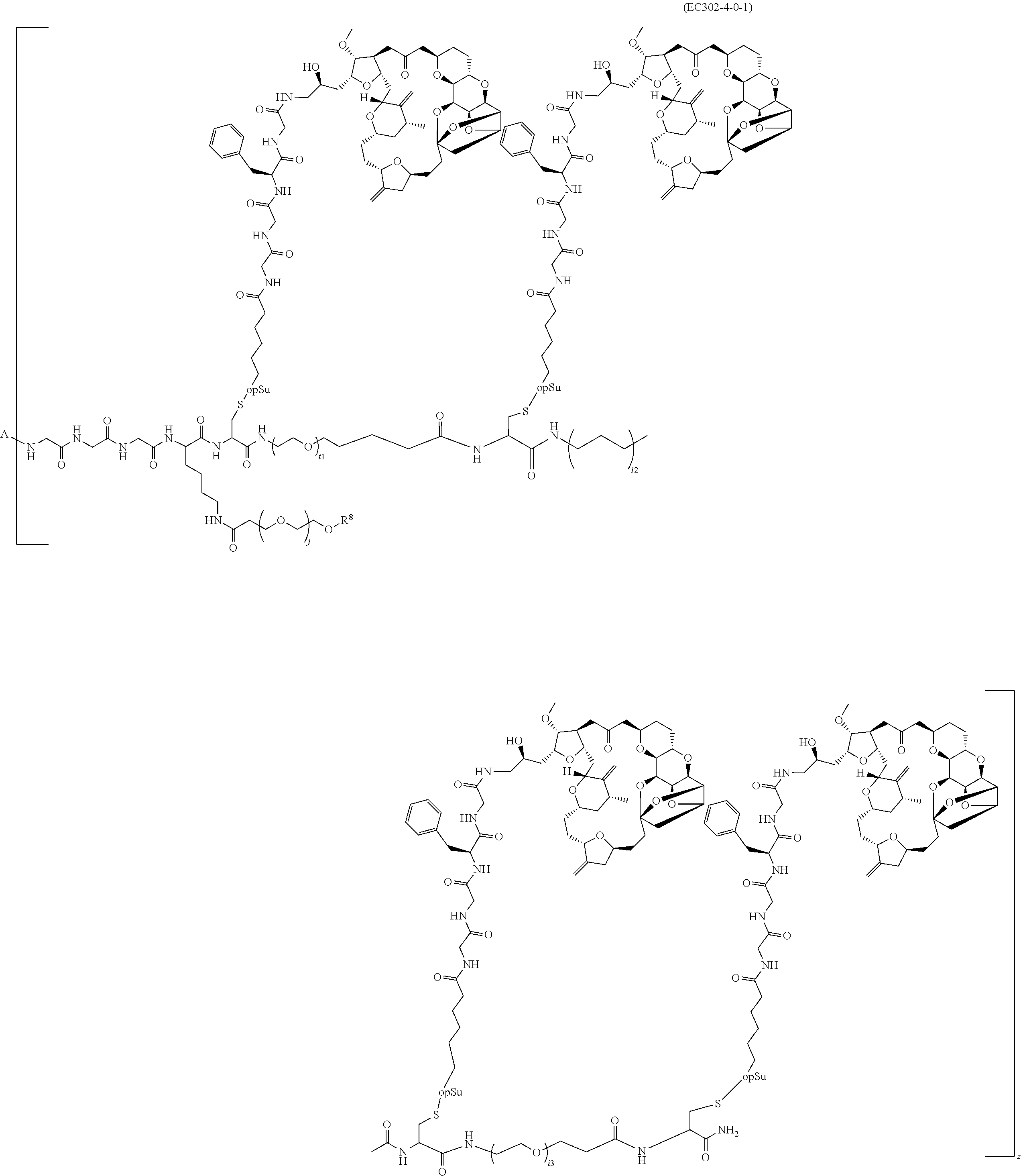

In an embodiment, i is 4. In an embodiment, conjugate EC301-2-0 is as follows (conjugate EC301-2-1-1):

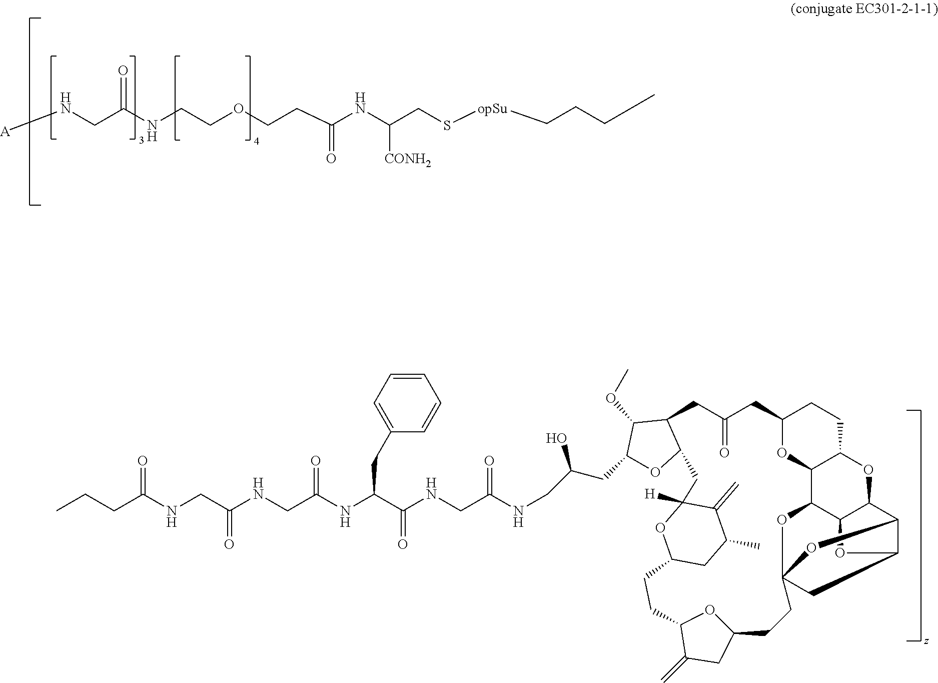

In an embodiment, i is 4, j is 8. In an embodiment, conjugate EC302-2-0 is as follows (conjugate EC302-2-1-1):

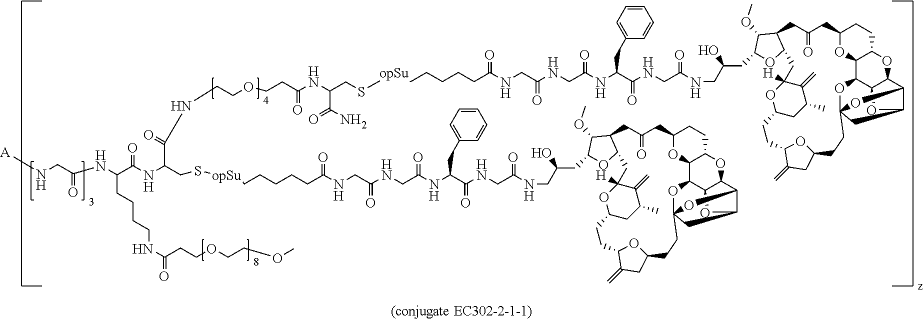

In an embodiment, i is 4, j is 12. In an embodiment, conjugate EC302-2-0 is as follows (conjugate EC302-2-4-1):

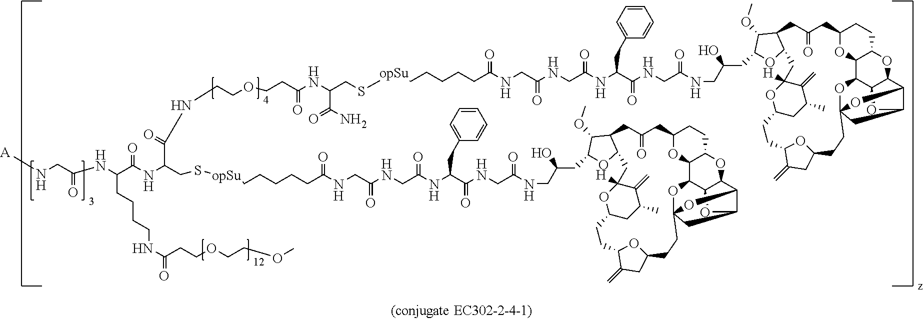

In one embodiment, n is 3, L² is —(CH₂)ₚ—(CH₂)₂(CO)—, p is 3, L¹ is Val-Cit-PABC. In one embodiment, conjugate EC301-1 has the structure of EC301-1-101:

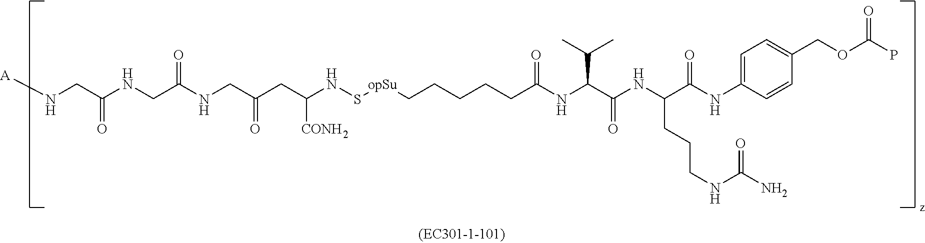

In one embodiment, conjugate EC301-2 has the structure of EC301-2-100:

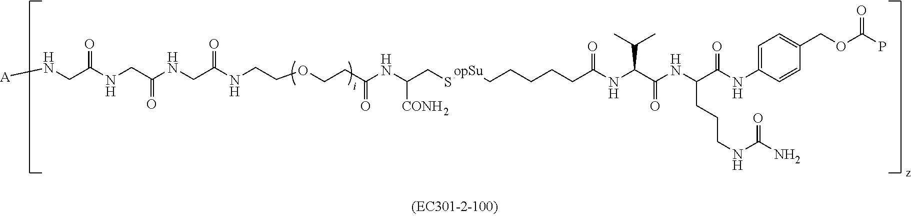

In one embodiment, conjugate EC301-3 has the structure of EC301-3-100:

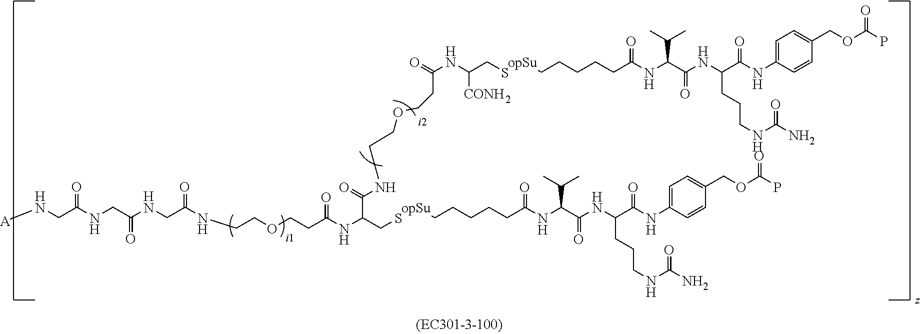

In one embodiment, conjugate EC301-4 has the structure of EC301-4-100:

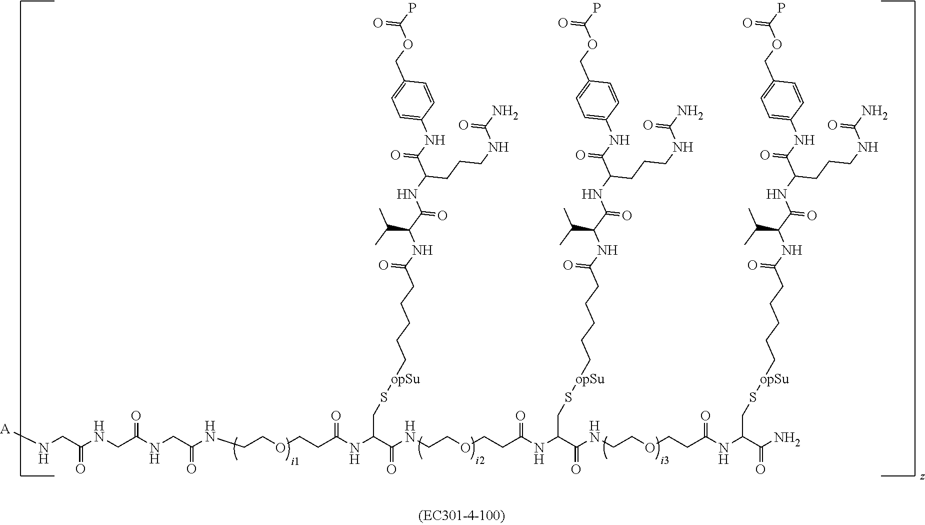

In one embodiment, conjugate EC301-5 has the structure of EC301-5-100:

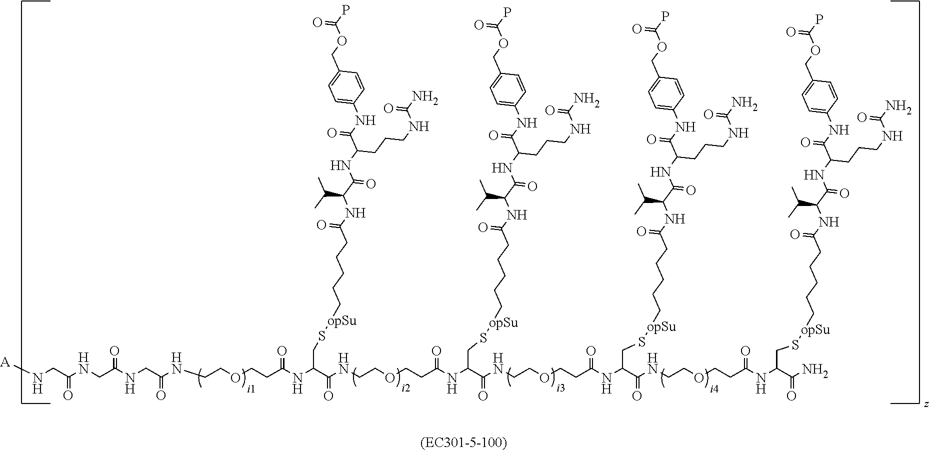

In one embodiment, conjugate EC302-1 has the structure of EC302-1-100:

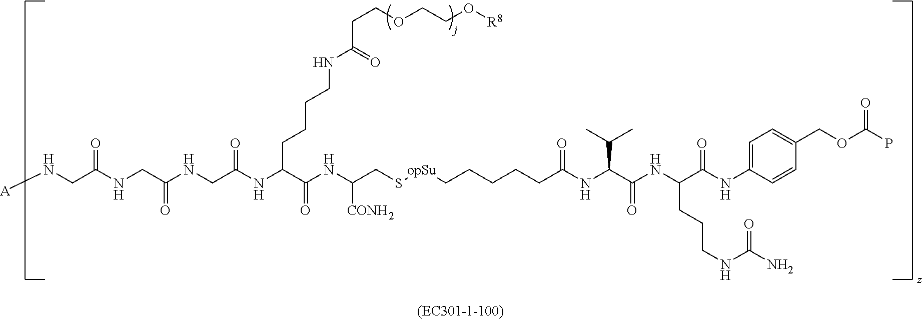

In one embodiment, conjugate EC302-2 has the structure of EC302-2-100:

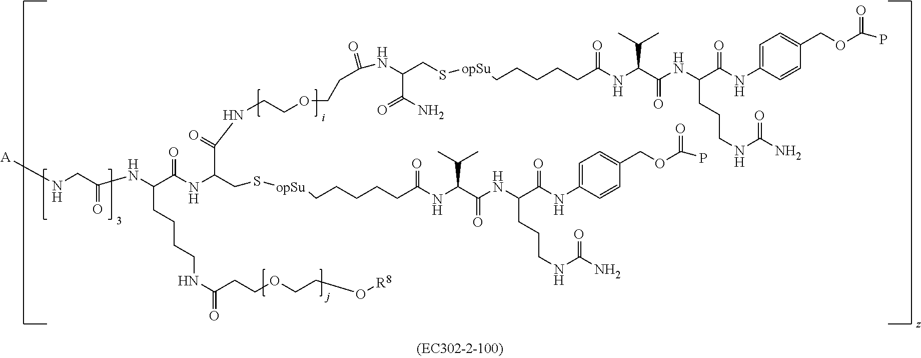

In one embodiment, conjugate EC302-3 has the structure of EC302-3-100:

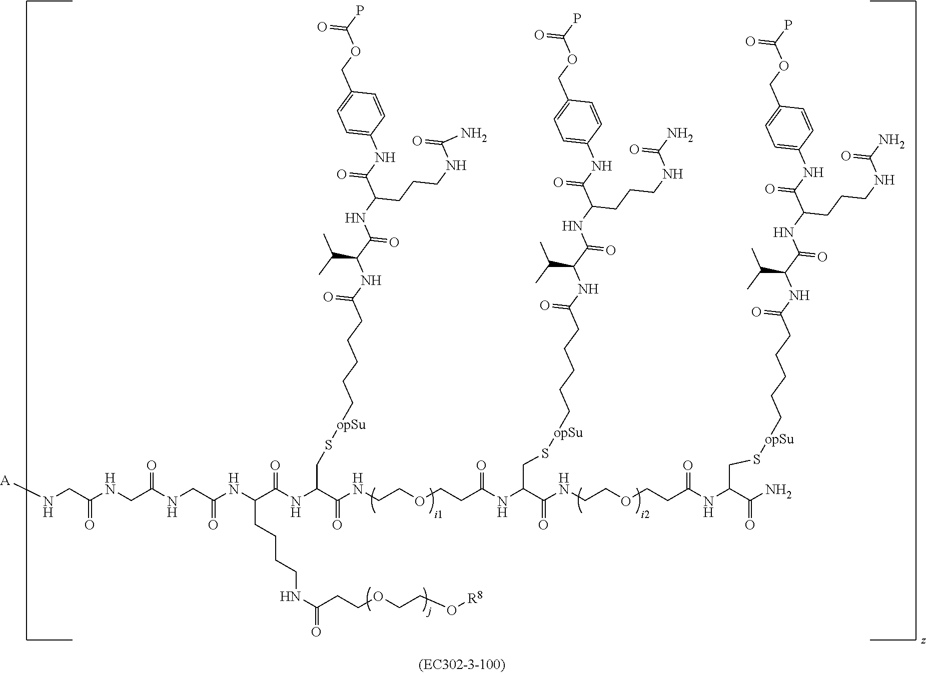

In one embodiment, conjugate EC302-4 has the structure of EC302-4-100:

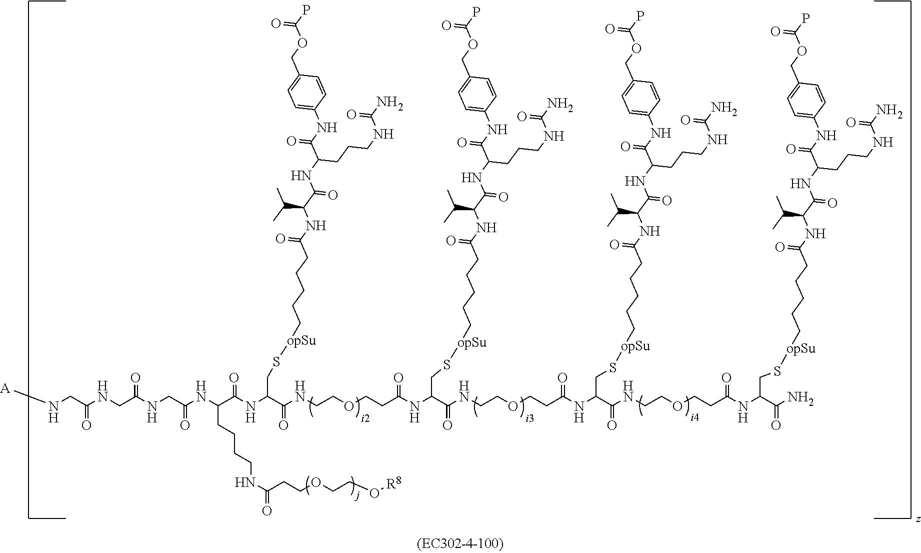

In one embodiment, conjugate EC301-1-101 has the structure of EC301-1-101-1:

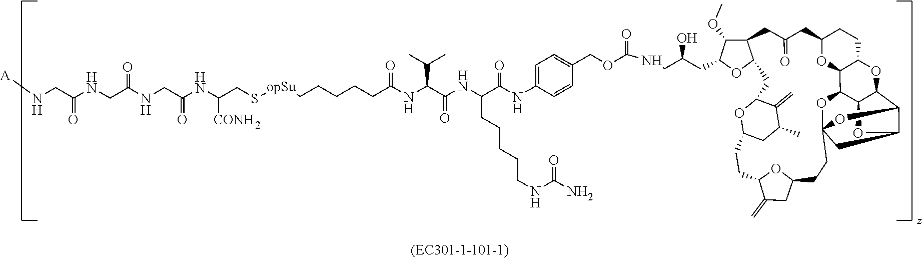

In one embodiment, conjugate EC301-2-100 has the structure of EC301-2-100-1:

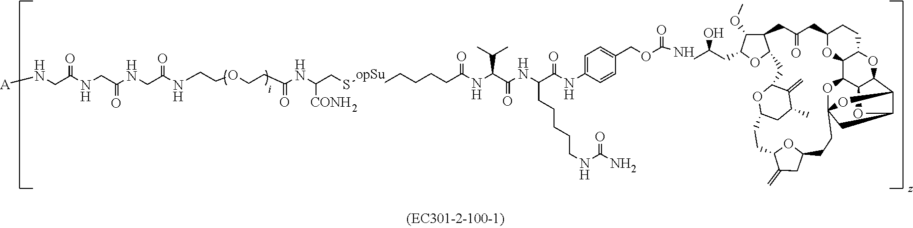

In one embodiment, conjugate EC301-3-100 has the structure of EC301-3-100-1:

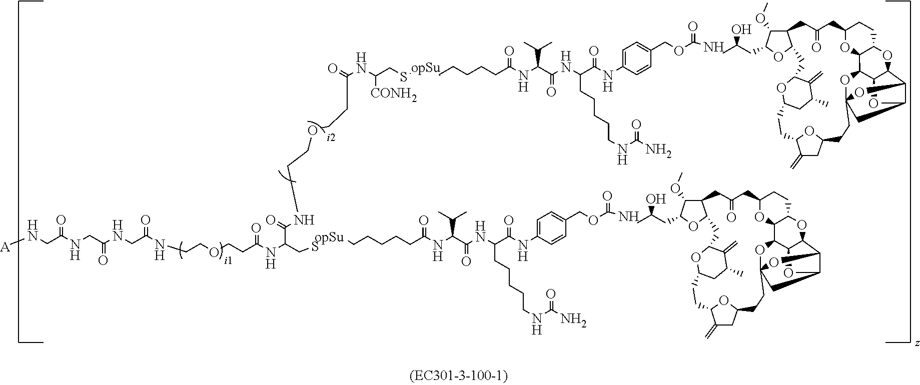

In one embodiment, conjugate EC301-4-100 has the structure of EC301-4-100-1:

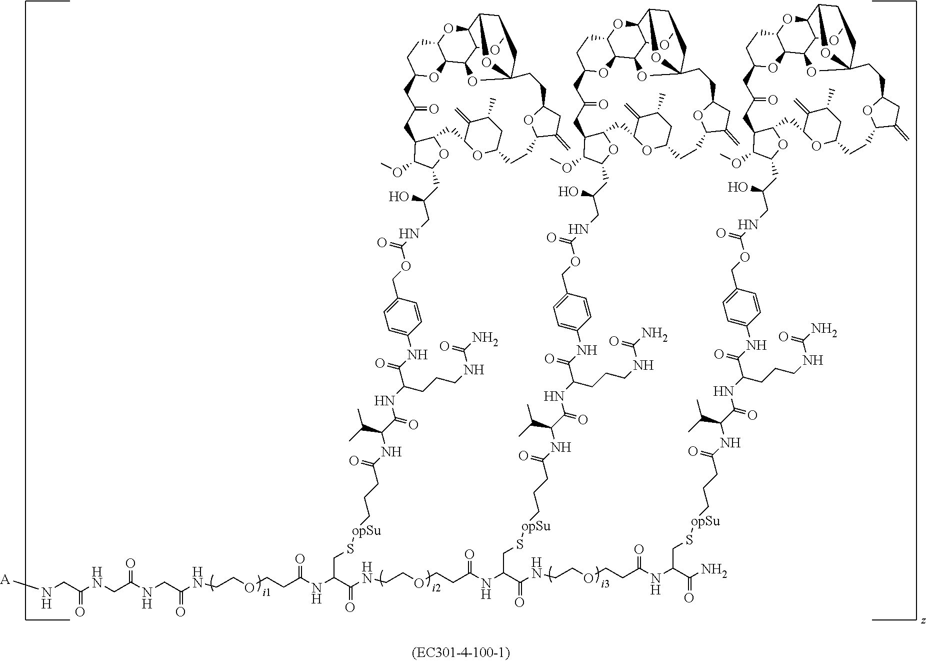

In one embodiment, conjugate EC301-5-100 has the structure of EC301-5-100-1:

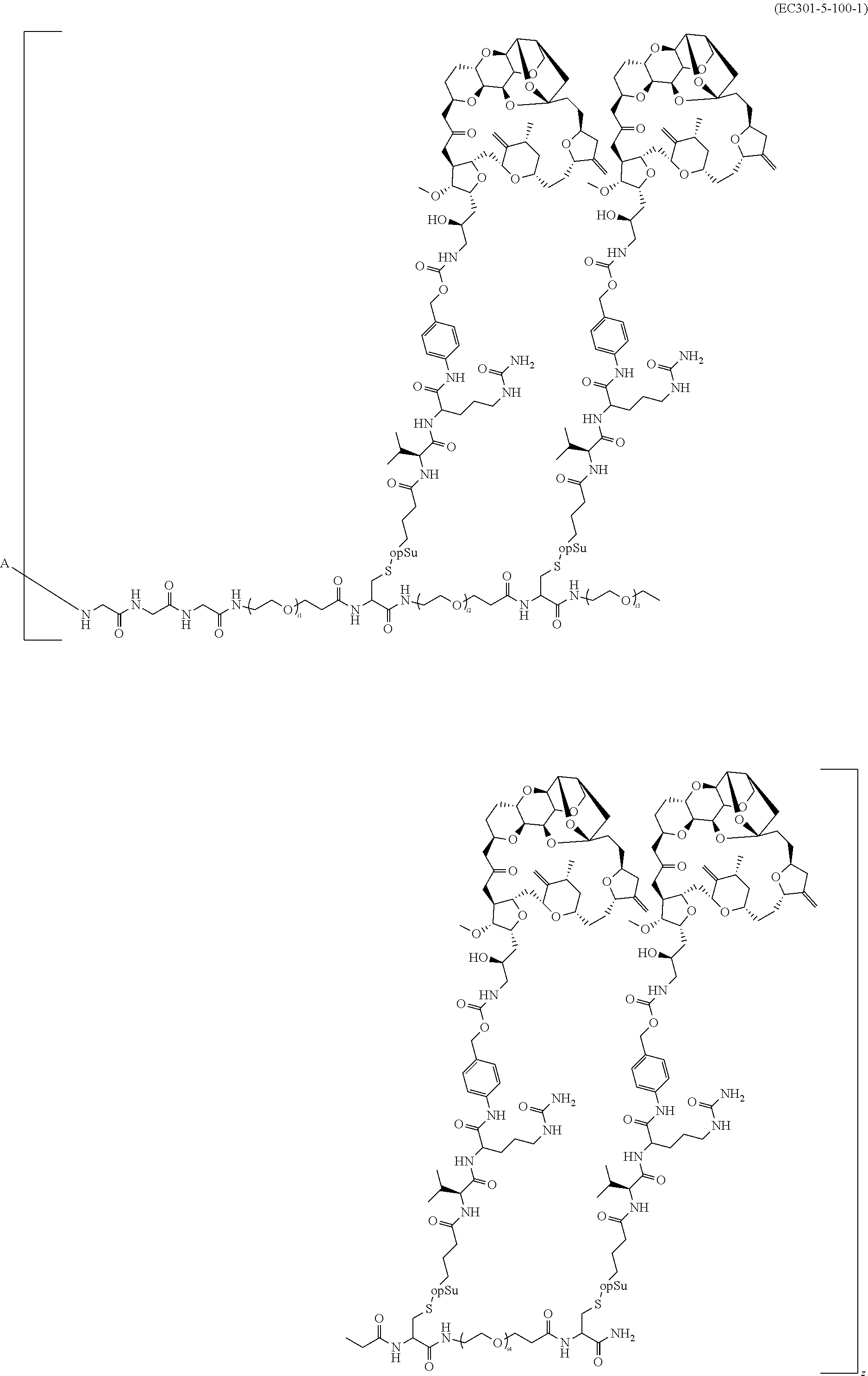

In one embodiment, conjugate EC302-1-100 has the structure of EC302-1-100-1:

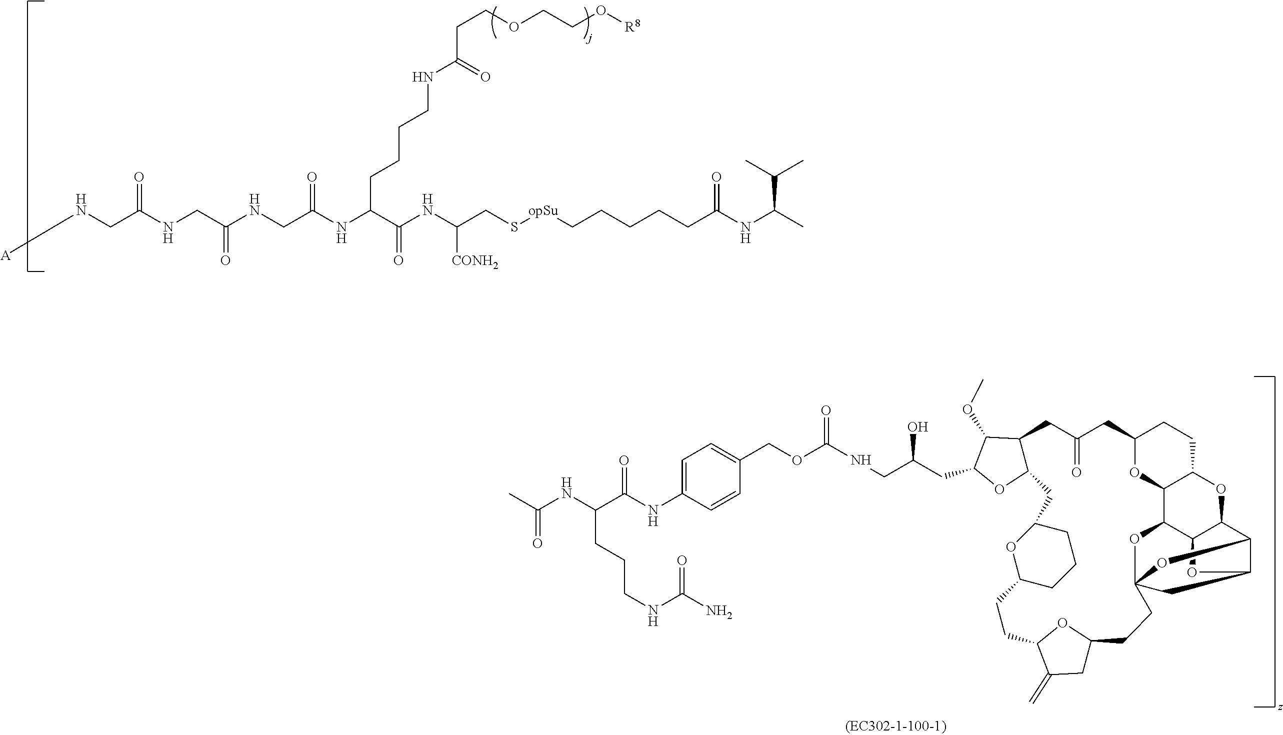

In one embodiment, conjugate EC302-2-100 has the structure of EC302-2-100-1:

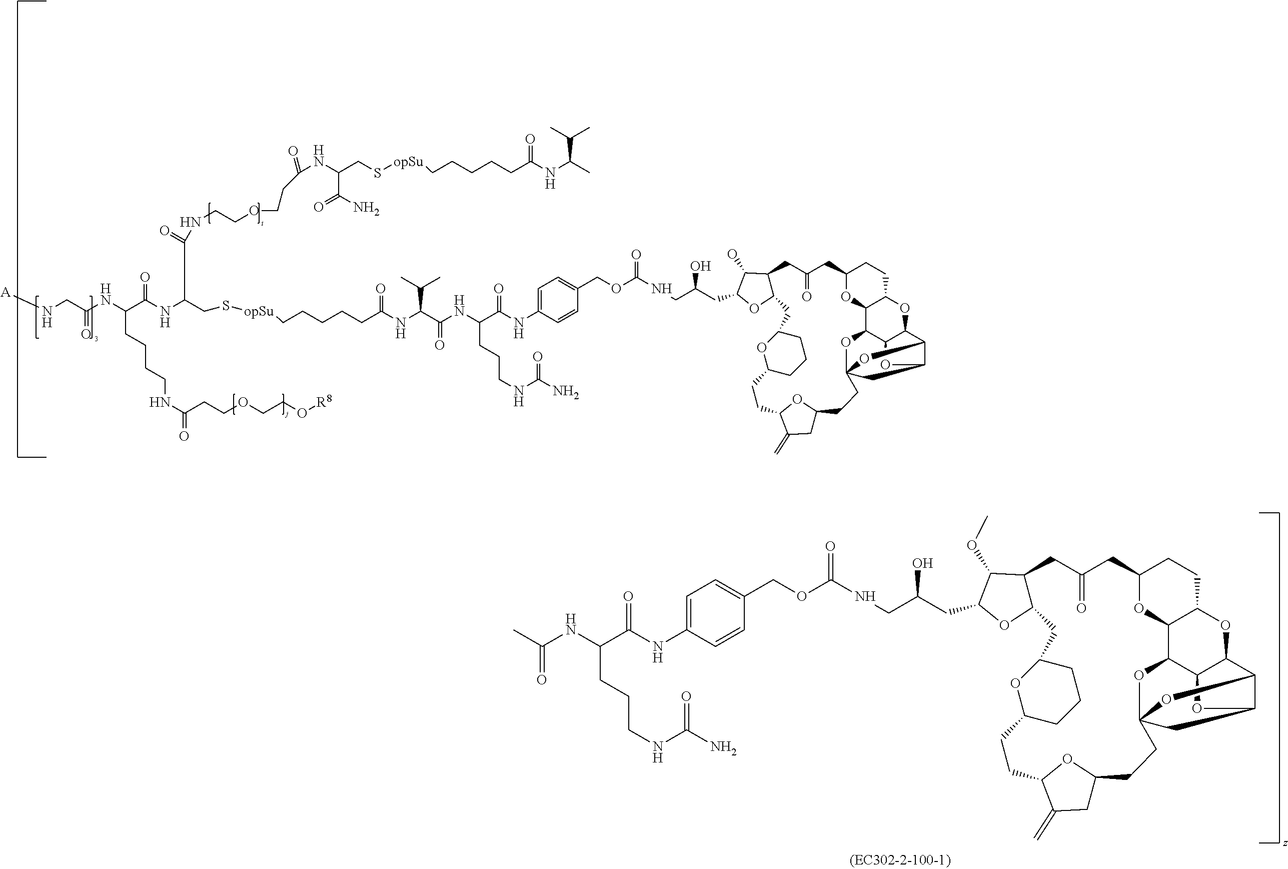

In one embodiment, conjugate EC302-3-100 has the structure of EC302-3-100-1:

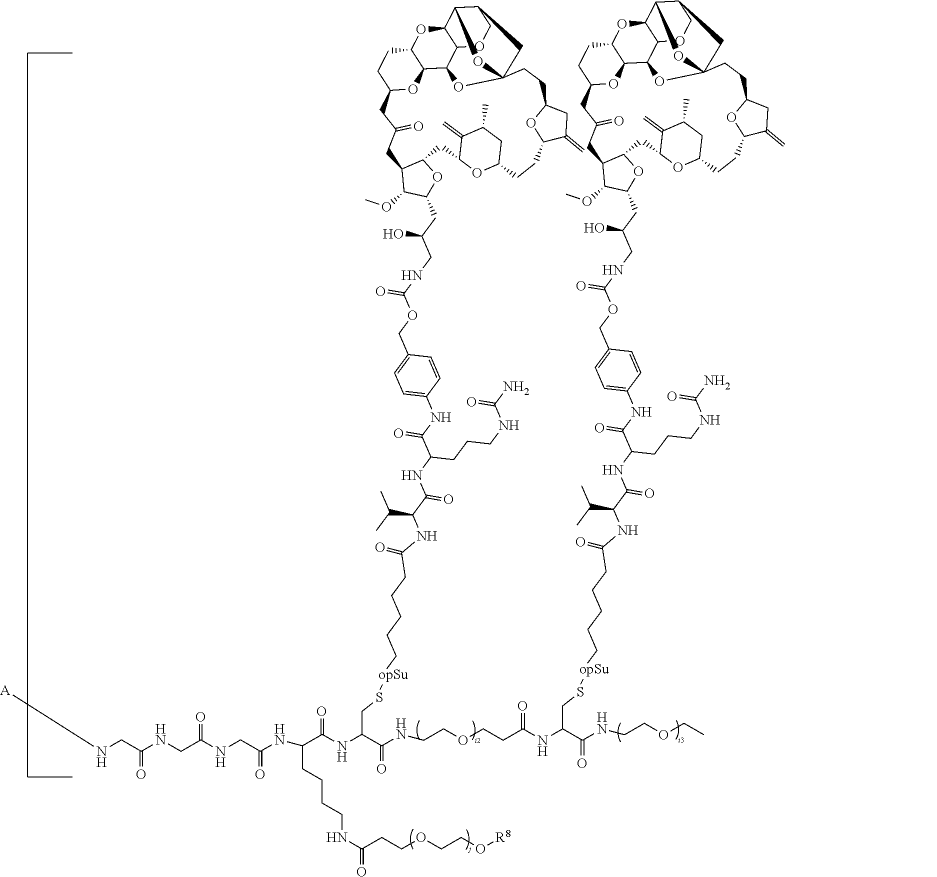

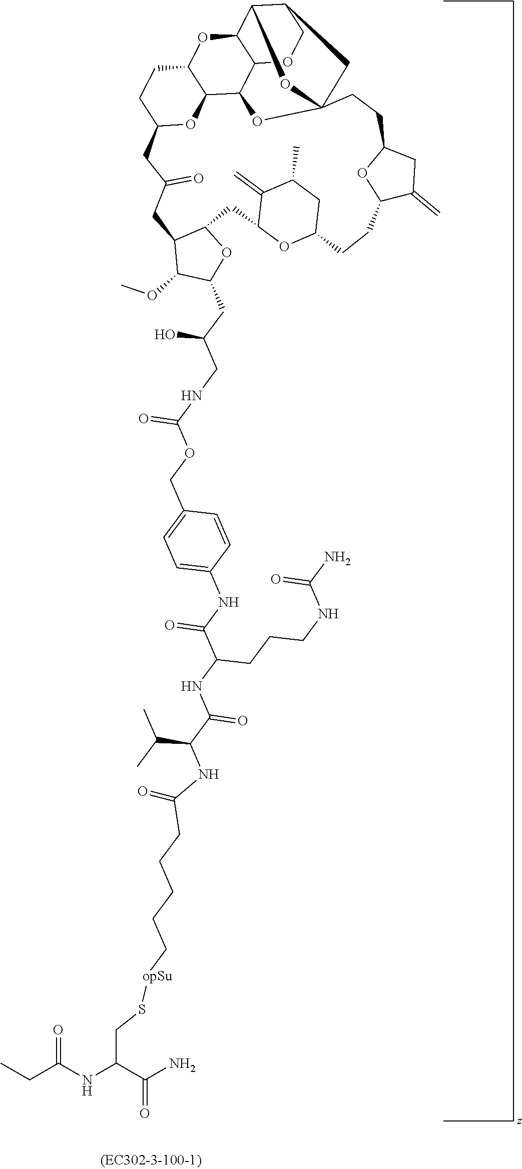

In one embodiment, conjugate EC302-4-100 has the structure of EC302-4-100-1:

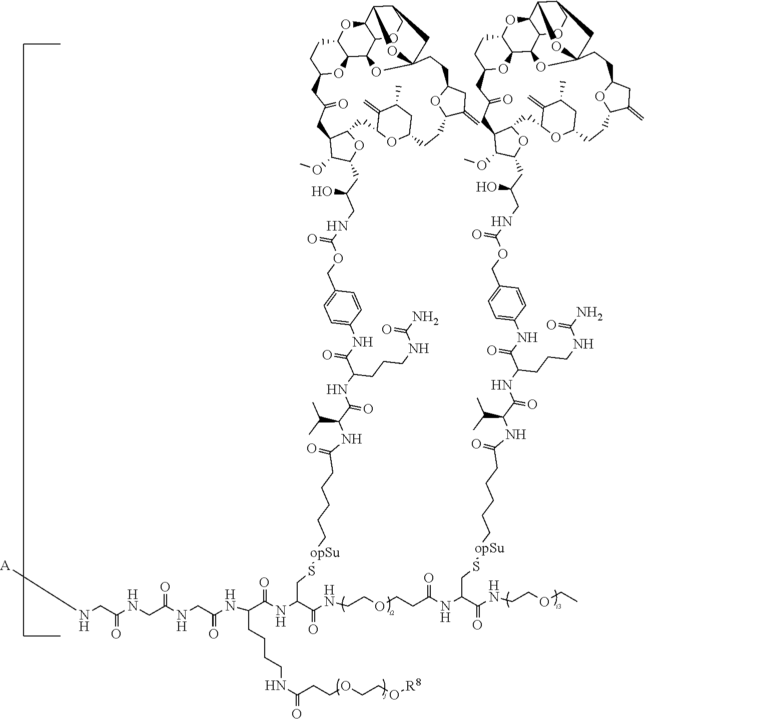

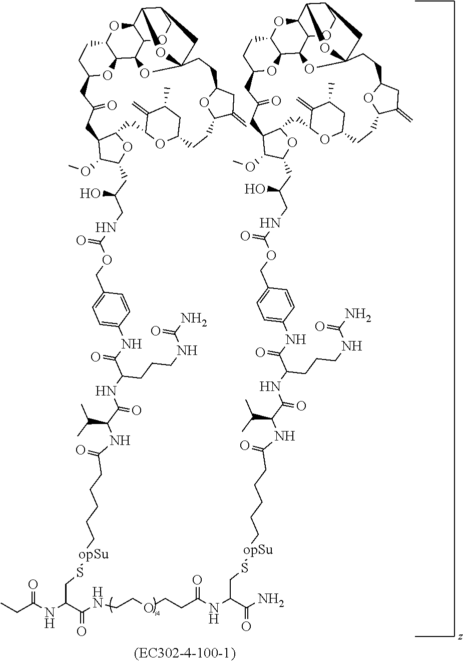

In an embodiment, i is 4. In an embodiment, conjugate EC301-2-100 is as follows (conjugate EC301-2-101-1):

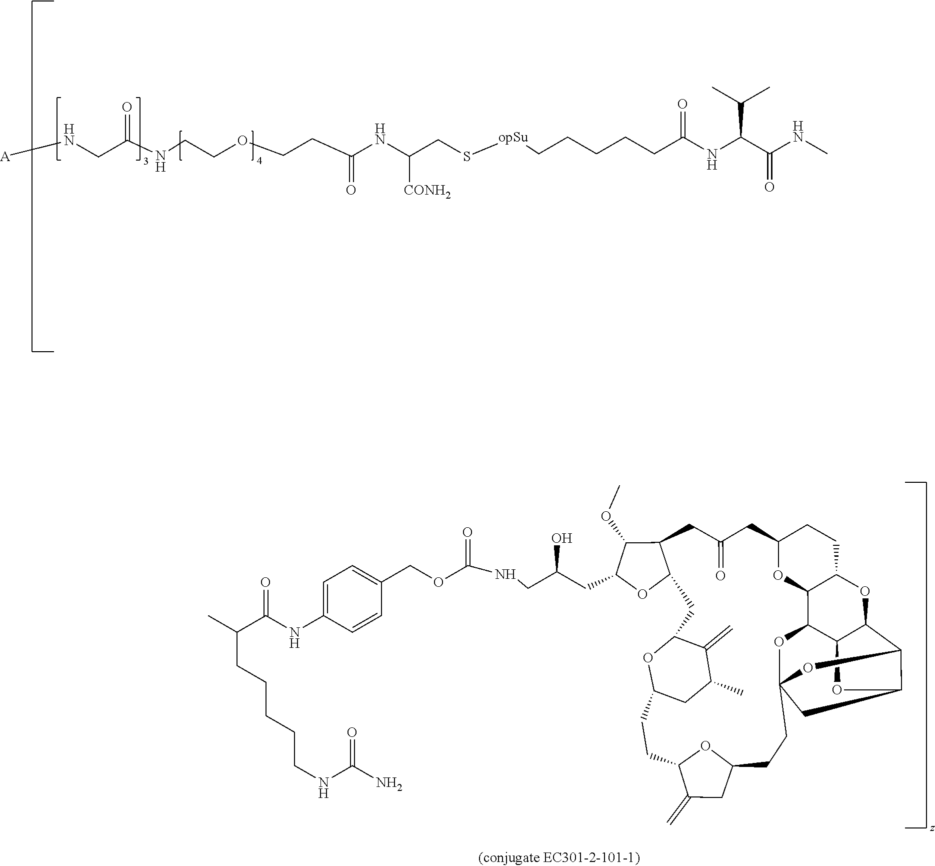

In an embodiment, i is 4, j is 8. In an embodiment, conjugate EC302-2-100 is as follows (conjugate EC302-2-101-1):

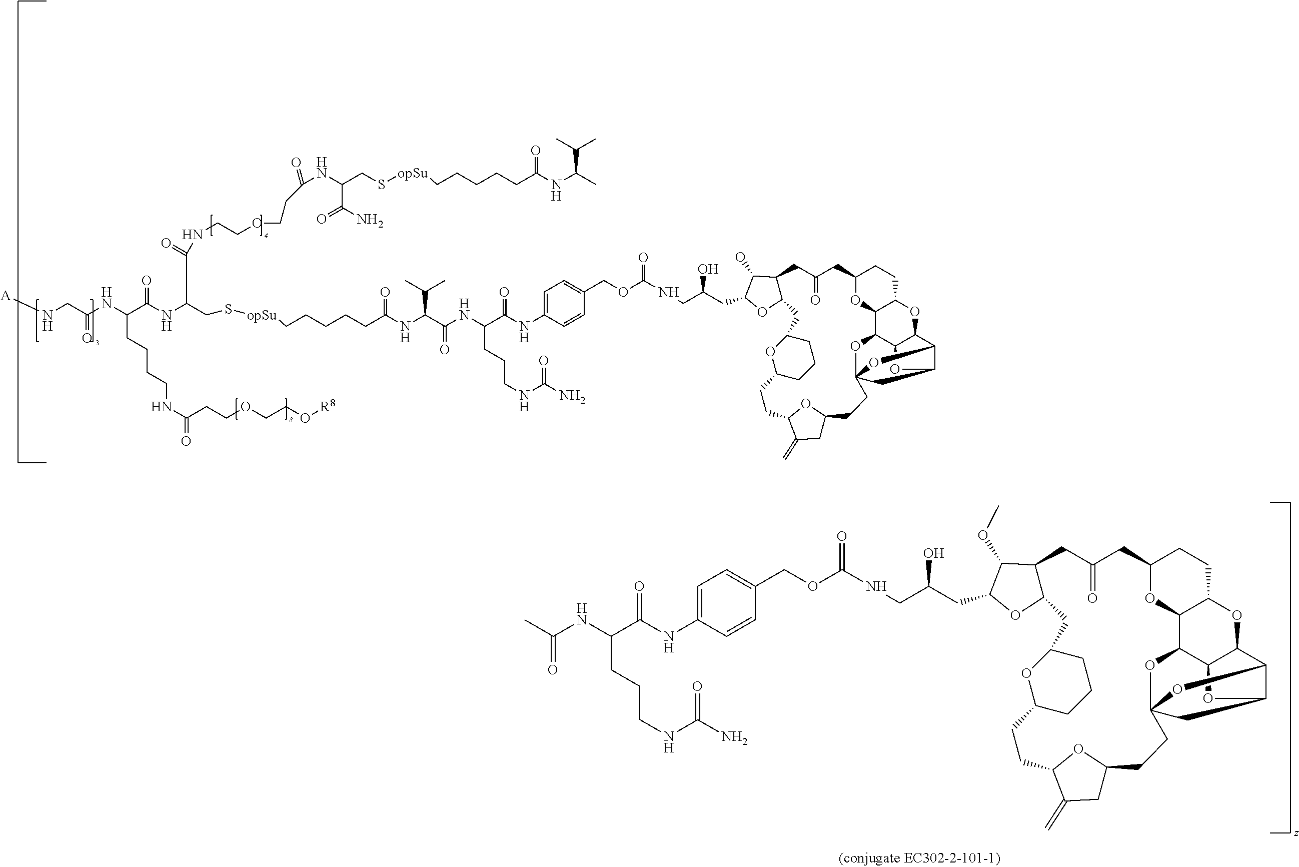

In an embodiment, i is 4, j is 12. In an embodiment, conjugate EC302-2-100 is as follows (conjugate EC302-2-104-1):

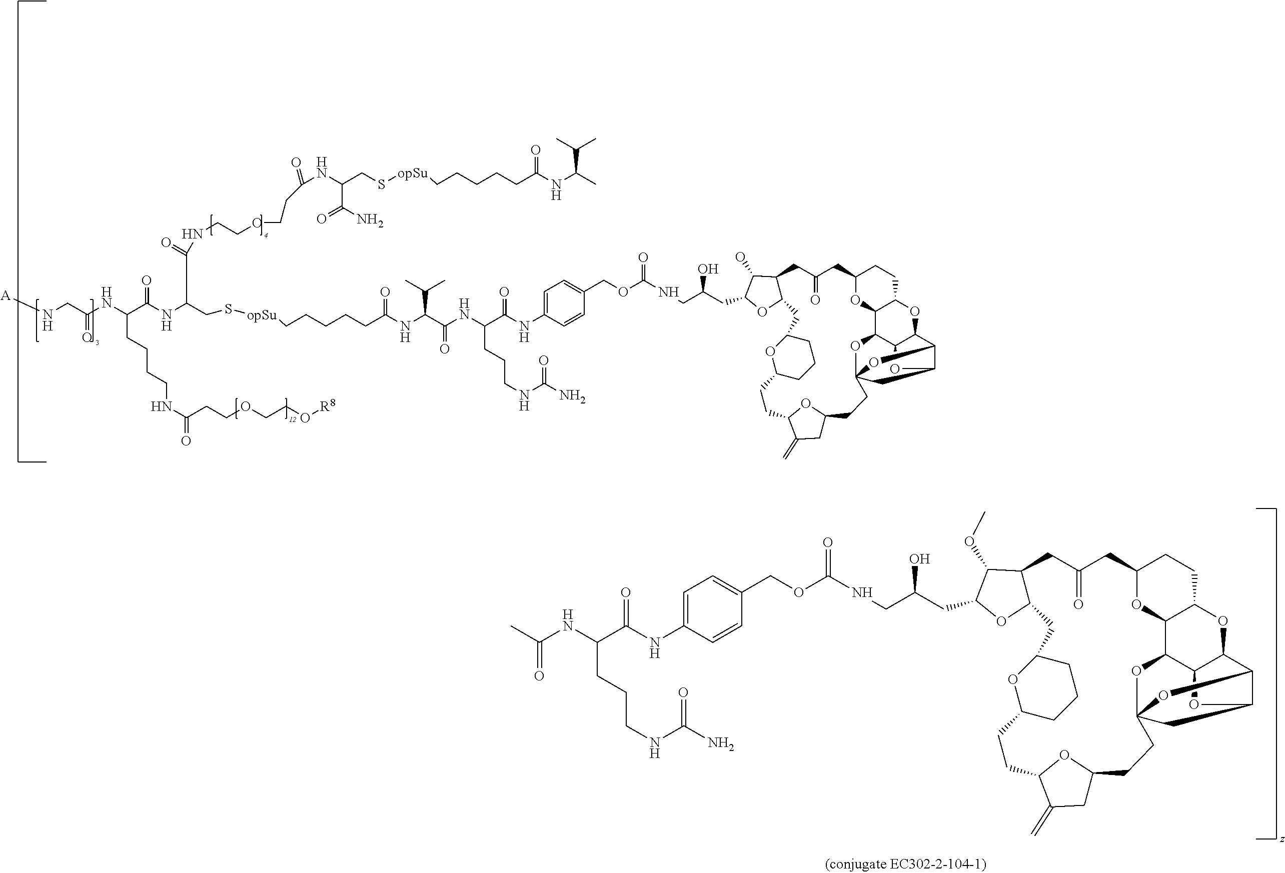

### Preparation of the Conjugate

The conjugates of the present disclosure can be prepared by any method known in the art. In some embodiments, the conjugate is prepared by the ligase-catalyzed site-specific conjugation of a targeting molecule and a compound of formula (II), wherein the targeting molecule is modified by a ligase recognition sequence. The method comprises step A and step B.

### Step A. Preparation of the Linker-Payload Compound

The compound of formula (II) of the present disclosure has defined structure, defined composition and high purity, so that when the conjugation reaction with an antibody is conducted, fewer impurities are introduced or no other impurities are introduced. When such a linker-payload compound is used for the ligase-catalyzed site-specific conjugation with a modified antibody containing a ligase recognition sequence, a homogeneous ADC with highly controllable quality is obtained.

### Step B. Linking the Targeting Molecule to Linker-Payload Compound

The targeting molecule of the present disclosure can be conjugated with the compound of formula (II) by any method known in the art.

The targeting molecule and the compound of formula (II) are linked to each other via the ligase-specific recognition sequences of the substrates. The recognition sequence depends on the particular ligase employed. In one embodiment, the targeting molecule is an antibody with recognition sequence-based terminal modifications introduced at the C-terminal of the light chain and/or the heavy chain, and the targeting molecule is conjugated with the compound of formula (II), under the catalysis of the wild type or optimized engineered ligase or any combination thereof, and under suitable catalytic reaction conditions.

In a specific embodiment, the ligase is Sortase A and the conjugation reaction can be represented by the following scheme:

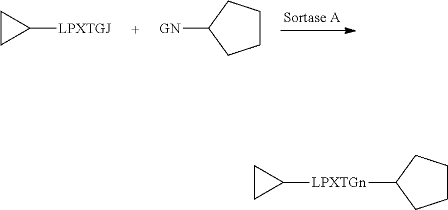

The triangle represents a portion of an antibody; and the pentagon represents a portion of a compound of formula (II). n, X and J are respectively as defined above. When conjugated with Gₙ, which is the corresponding recognition sequence of the acceptor substrate, the upstream peptide bond of the glycine in the LPXTGJ sequence is cleaved by Sortase A, and the resulting intermediate is linked to the free N-terminal of Gₙ to generate a new peptide bond. The resulting amino acid sequence is LPXTGₙ. The sequences Gₙ and LPXTGJ are as defined above.

### Metabolism of the Conjugate in a Physiological Environment

The linker-payload compound can be cleaved, especially at the linker structure. When a part or the whole linker is cleaved in tumor cells, the antitumor compound moiety is released to exhibit the antitumor effect of the antitumor compound. As the linker is cleaved at a connecting position to the drug, the antitumor compound is released in its intrinsic structure to exhibit its intrinsic antitumor effect.

In an embodiment, Cleavable sequence 1 (such as GGFG or Val-Cit-PABC) can be cleaved by lysosomal enzymes (such as cathepsin B and/or cathepsin L).

In one embodiment, the payload is a cytotoxin or a fragment thereof.

In one embodiment, the antibody-drug conjugate (numbered as ECx) is as shown in the following Table 2.

TABLE 2
Specific conjugates

| | Linker-Payload | | A (targeting |
|---|---|---|---|
| ADC | formula | Palyload | molecule) |
| EC301-1-1-1-1 | EB301-1-1-1 | formula (ii) | Ab0036 |
| EC301-1-1-1-2 | EB301-1-1-1 | formula (ii) | Ab0206 |
| EC301-1-1-1-3 | EB301-1-1-1 | formula (ii) | Ab0001 |
| EC301-2-1-1-1 | EB301-2-1-1 | formula (ii) | Ab0036 |
| EC301-2-1-1-2 | EB301-2-1-1 | formula (ii) | Ab0206 |
| EC301-2-1-1-3 | EB301-2-1-1 | formula (ii) | Ab0001 |
| EC301-3-1-1-1 | EB301-3-1-1 | formula (ii) | Ab0036 |
| EC301-3-1-1-2 | EB301-3-1-1 | formula (ii) | Ab0206 |
| EC301-3-1-1-3 | EB301-3-1-1 | formula (ii) | Ab0001 |
| EC301-4-1-1-1 | EB301-4-1-1 | formula (ii) | Ab0036 |
| EC301-4-1-1-2 | EB301-4-1-1 | formula (ii) | Ab0206 |
| EC301-4-1-1-3 | EB301-4-1-1 | formula (ii) | Ab0001 |
| EC301-5-1-1-1 | EB301-5-1-1 | formula (ii) | Ab0036 |
| EC301-5-1-1-2 | EB301-5-1-1 | formula (ii) | Ab0206 |
| EC301-5-1-1-3 | EB301-5-1-1 | formula (ii) | Ab0001 |
| EC302-1-1-1-1 | EB302-1-1-1 | formula (ii) | Ab0036 |
| EC302-1-1-1-2 | EB302-1-1-1 | formula (ii) | Ab0206 |
| EC302-1-1-1-3 | EB302-1-1-1 | formula (ii) | Ab0001 |
| EC302-1-4-1-1 | EB302-1-4-1 | formula (ii) | Ab0036 |
| EC302-1-4-1-2 | EB302-1-4-1 | formula (ii) | Ab0206 |
| EC302-1-4-1-3 | EB302-1-4-1 | formula (ii) | Ab0001 |
| EC302-2-1-1-1 | EB302-2-1-1 | formula (ii) | Ab0036 |
| EC302-2-1-1-2 | EB302-2-1-1 | formula (ii) | Ab0206 |
| EC302-2-1-1-3 | EB302-2-1-1 | formula (ii) | Ab0001 |
| EC302-2-4-1-1 | EB302-2-4-1 | formula (ii) | Ab0036 |
| EC302-2-4-1-2 | EB302-2-4-1 | formula (ii) | Ab0206 |
| EC302-2-4-1-3 | EB302-2-4-1 | formula (ii) | Ab0001 |
| EC302-3-1-1-1 | EB302-3-1-1 | formula (ii) | Ab0036 |
| EC302-3-1-1-2 | EB302-3-1-1 | formula (ii) | Ab0206 |
| EC302-3-1-1-3 | EB302-3-1-1 | formula (ii) | Ab0001 |
| EC302-3-4-1-1 | EB302-3-4-1 | formula (ii) | Ab0036 |
| EC302-3-4-1-2 | EB302-3-4-1 | formula (ii) | Ab0206 |
| EC302-3-4-1-3 | EB302-3-4-1 | formula (ii) | Ab0001 |
| EC302-4-1-1-1 | EB302-4-1-1 | formula (ii) | Ab0036 |
| EC302-4-1-1-2 | EB302-4-1-1 | formula (ii) | Ab0206 |
| EC302-4-1-1-3 | EB302-4-1-1 | formula (ii) | Ab0001 |
| EC302-4-4-1-1 | EB302-4-4-1 | formula (ii) | Ab0036 |
| EC302-4-4-1-2 | EB302-4-4-1 | formula (ii) | Ab0206 |
| EC302-4-4-1-3 | EB302-4-4-1 | formula (ii) | Ab0001 |
| EC301-1-101-1-1 | EB301-1-101-1 | formula (ii) | Ab0036 |
| EC301-1-101-1-2 | EB301-1-101-1 | formula (ii) | Ab0206 |
| EC301-1-101-1-3 | EB301-1-101-1 | formula (ii) | Ab0001 |
| EC301-2-101-1-1 | EB301-2-101-1 | formula (ii) | Ab0036 |
| EC301-2-101-1-2 | EB301-2-101-1 | formula (ii) | Ab0206 |
| EC301-2-101-1-3 | EB301-2-101-1 | formula (ii) | Ab0001 |
| EC301-3-101-1-1 | EB301-3-101-1 | formula (ii) | Ab0036 |
| EC301-3-101-1-2 | EB301-3-101-1 | formula (ii) | Ab0206 |
| EC301-3-101-1-3 | EB301-3-101-1 | formula (ii) | Ab0001 |
| EC301-4-101-1-1 | EB301-4-101-1 | formula (ii) | Ab0036 |
| EC301-4-101-1-2 | EB301-4-101-1 | formula (ii) | Ab0206 |
| EC301-4-101-1-3 | EB301-4-101-1 | formula (ii) | Ab0001 |
| EC301-5-101-1-1 | EB301-5-101-1 | formula (ii) | Ab0036 |
| EC301-5-101-1-2 | EB301-5-101-1 | formula (ii) | Ab0206 |
| EC301-5-101-1-3 | EB301-5-101-1 | formula (ii) | Ab0001 |
| EC302-1-101-1-1 | EB302-1-101-1 | formula (ii) | Ab0036 |
| EC302-1-101-1-2 | EB302-1-101-1 | formula (ii) | Ab0206 |
| EC302-1-101-1-3 | EB302-1-101-1 | formula (ii) | Ab0001 |
| EC302-1-104-1-1 | EB302-1-104-1 | formula (ii) | Ab0036 |
| EC302-1-104-1-2 | EB302-1-104-1 | formula (ii) | Ab0206 |
| EC302-1-104-1-3 | EB302-1-104-1 | formula (ii) | Ab0001 |
| EC302-2-101-1-1 | EB302-2-101-1 | formula (ii) | Ab0036 |
| EC302-2-101-1-2 | EB302-2-101-1 | formula (ii) | Ab0206 |
| EC302-2-101-1-3 | EB302-2-101-1 | formula (ii) | Ab0001 |
| EC302-2-104-1-1 | EB302-2-104-1 | formula (ii) | Ab0036 |
| EC302-2-104-1-2 | EB302-2-104-1 | formula (ii) | Ab0206 |
| EC302-2-104-1-3 | EB302-2-104-1 | formula (ii) | Ab0001 |
| EC302-3-101-1-1 | EB302-3-101-1 | formula (ii) | Ab0036 |
| EC302-3-101-1-2 | EB302-3-101-1 | formula (ii) | Ab0206 |
| EC302-3-101-1-3 | EB302-3-101-1 | formula (ii) | Ab0001 |
| EC302-3-104-1-1 | EB302-3-104-1 | formula (ii) | Ab0036 |
| EC302-3-104-1-2 | EB302-3-104-1 | formula (ii) | Ab0206 |
| EC302-3-104-1-3 | EB302-3-104-1 | formula (ii) | Ab0001 |
| EC302-4-101-1-1 | EB302-4-101-1 | formula (ii) | Ab0036 |
| EC302-4-101-1-2 | EB302-4-101-1 | formula (ii) | Ab0206 |
| EC302-4-101-1-3 | EB302-4-101-1 | formula (ii) | Ab0001 |
| EC302-4-104-1-1 | EB302-4-104-1 | formula (ii) | Ab0036 |
| EC302-4-104-1-2 | EB302-4-104-1 | formula (ii) | Ab0206 |
| EC302-4-104-1-3 | EB302-4-104-1 | formula (ii) | Ab0001 |

Note:
When the above listed ADC is formed by the corresponding above-listed linker-payload and the corresponding above-listed antibody (targeting molecule), the upstream peptide bond of GG in the LPETGG sequence in the antibody is cleaved by Sortase A, and the resulting intermediate is linked to the free N-terminal of G₃ in linker-payload to generate a new peptide bond, thereby forming the resulting amino acid sequence LPXTG₃.

### Pharmaceutical Composition and Pharmaceutical Preparation

Another object of the disclosure is to provide a pharmaceutical composition comprising a prophylactically or therapeutically effective amount of a conjugate of the present disclosure, and at least one pharmaceutically acceptable carrier.

The pharmaceutical composition of the present disclosure may be administered in any manner as long as it achieves the effect of preventing, alleviating, preventing or curing the symptoms of a human or animal. For example, various suitable dosage forms can be prepared according to the administration route, especially injections such as lyophilized powder for injection, injection, or sterile powder for injection.

The term “pharmaceutically acceptable” means that when contacted with tissues of the patient within the scope of normal medical judgment, no undue toxicity, irritation or allergic reaction, etc. shall arise, having reasonable advantage-disadvantage ratios and effective for the intended use.

The term pharmaceutically acceptable carrier refers to those carrier materials which are pharmaceutically acceptable and which do not interfere with the bioactivities and properties of the conjugate. Examples of aqueous carriers include but are not limited to buffered saline, and the like. The pharmaceutically acceptable carrier also includes carrier materials which brings the composition close to physiological conditions, such as pH adjusting agents, buffering agents, toxicity adjusting agents and the like, and sodium acetate, sodium chloride, potassium chloride, calcium chloride, sodium lactate, and the like.

In one embodiment, the pharmaceutical composition of the present disclosure has a drug to antibody ratio (DAR) of an integer or non-integer of about 1 to about 20, such as about 1 to about 10, about 1 to about 8, about 1 to about 6, about 2 to about 5, about 2 to about 4, or about 3 to about 4. In a particular embodiment, the conjugate of the present disclosure has a DAR of about 2, about 4, about 6 or about 8. In a particular embodiment, the conjugate EC302-2 has a DAR of about 2.8 to about 3.6; especially about 2.9 to about 3.1, about 3.1 to about 3.2, or about 3.3 to about 3.5. In a particular embodiment, the conjugate EC301-2 has a DAR of about 1.6 to about 2, preferably about 1.7 to about 2. In a particular embodiment, the conjugate EC301-2 has a DAR of about 1.7 to about 1.8, or about 1.8 to about 1.9.

### Treatment Method and Use

The conjugates of the present disclosure are useful for the treatment of tumors and/or autoimmune diseases. Tumors susceptible to conjugate treatment include those characterized by specific tumor-associated antigens or cell surface receptors, and those will be recognized by the targeting molecule in the conjugate and can be killed by the payload/cytotoxin in the conjugate.

Accordingly, in yet another aspect, also provided is use of a conjugate of the present disclosure or a pharmaceutical composition of the present disclosure in the manufacture of a medicament for treating a disease, disorder or condition selected from a tumor or an autoimmune disease.

In another aspect, provided is a conjugate of the present disclosure or a pharmaceutical composition of the present disclosure for use in the treatment of a tumor or an autoimmune disease.

In a further aspect, provided is a method of treating a tumor or an autoimmune disease, the method comprising administering to an individual in need thereof an effective amount of a conjugate of the present disclosure or a pharmaceutical composition of the present disclosure.

In a preferred embodiment, the conjugate of the present disclosure formed by conjugation of the anti-HER2 antibody and the small molecule cytotoxin can specifically bind to HER2 on the surface of the tumor cell and selectively kill the HER2-expressing tumor cells. In another preferred embodiment, the anti-HER2 antibody is an anti-human HER2 antibody. In another preferred embodiment, provided is use of a conjugate of the present disclosure or a pharmaceutical composition of the present disclosure in the manufacture of a medicament for treating a disease, disorder or condition selected from HER2-positive tumors. In a more preferred embodiment, the disease, disorder or condition is selected from the group consisting of breast cancer, gastric cancer, lung cancer, ovarian cancer, urothelial cancer, and the like.

In a preferred embodiment, the conjugate of the present disclosure formed by conjugation of the anti-FGFR3 antibody and the small molecule cytotoxin can specifically bind to FGFR3 on the surface of the tumor cell and selectively kill the FGFR3-expressing tumor cells. In another preferred embodiment, the anti-FGFR3 antibody is an anti-human FGFR3 antibody. In another preferred embodiment, provided is use of a conjugate of the present disclosure or a pharmaceutical composition of the present disclosure in the manufacture of a medicament for treating a disease, disorder or condition selected from FGFR3-positive tumors. In a more preferred embodiment, the disease, disorder or condition is selected from the group consisting of multiple myeloma, bladder cancer, urothelial cancer, glioblastoma, and the like.

The dosage of the conjugate administered to the subject can be adjusted to a considerable extent. The dosage can vary according to the particular route of administration and the needs of the subject, and can be subjected to the judgment of the health care professional.

### Beneficial Effects

The antibody-drug conjugate of the present invention uses specially designed linker-payload, and is stable and can achieve great efficacy with suitable DAR, and therefore can reduce side effects and increase the therapeutic index.

The present disclosure utilizes a linker-payload compound with unique structure and uses a ligase to catalyze the conjugation of the targeting molecule and the linker-payload compound. The conjugate of the present disclosure has good homogeneity, high activity and high selectivity. Furthermore, the toxicity of the linker-payload compound is much lower than that of the free payload, and thus the manufacture process of the drug is less detrimental, which is advantageous for industrial production.

The conjugate of the present disclosure achieves at least one of the following technical effects:
(1) High inhibitory activity against target cells, and strong bystander killing effect.
(2) Good physicochemical properties (e.g., solubility, physical and/or chemical stability).
(3) Good pharmacokinetic properties (e.g., good stability in plasma, appropriate half-life and duration of action).
(4) Good safety (low toxicity on non-target normal cells or tissues, and/or fewer side effects, wider treatment window), etc.
(5) Highly modular design, simple assembly of multiple drugs.

### EXAMPLES

### Preparation Example

In order to more clearly illustrate the objects and technical solutions, the present disclosure is further described below with reference to specific examples. It is to be understood that the examples are not intended to limit the scope of the disclosure. The specific experimental methods which were not mentioned in the following examples were carried out according to conventional experimental method.

### Instruments, Materials and Reagents

Unless otherwise stated, the instruments and reagents used in the examples are commercially available. The reagents can be used directly without further purification.
MS: Thermo Fisher Q Exactive Plus, Water2795-Quattro micro triple quadrupole mass spectrometer
HPLC: Waters 2695, Agilent 1100, Agilent 1200
Semi-preparative HPLC: Lisure HP plus 50D
Flow Cytometry: CytoFLEX S
HIC-HPLC: Butyl-HIC; mobile phase A: 25 mM PB, 2M (NH₄)₂SO₄, pH 7.0; mobile phase B: 25 mM PB, pH 7.0; flow rate: 0.8 ml/min; acquisition time: 25 min; injection amount: 20 μg; column temperature: 25° C.; detection wavelength: 280 nm; sample chamber temperature: 8° C.
SEC-HPLC: column: TSK-gel G3000 SWXL, TOSOH 7.8 mm ID×300 mm, 5 μm; mobile phase: 0.2 M KH₂PO₄, 0.25 M KCl, pH 6.2; flow rate: 0.5 ml/min; acquisition time: 30 min; injection volume: 50 μl; column temperature: 25° C.; detection wavelength; 280 nm; sample tray temperature: 8° C.

CHO was obtained from Thermo Fisher Scientific; pcDNA 3.3 was obtained from Life Technology; HEK293F was obtained from Prejin; PEIMAX transfection reagent was obtained from Polyscience; MabSelect Sure ProA was obtained from GE; Capto S ImpAct was obtained from GE; Rink-amide-MBHA-resin and dichloro resin were obtained from Nankai synthesis; HCC1954 was obtained from ATCC CAT #CRL-2338; SK-BR-3 was obtained from ATCC CAT #HTB-30; BT-474 was obtained from ATCC CAT #HTB-20; NCI-N87 cells was obtained from ATCC CAT #CRL-5822; MCF-7 was obtained from ATCC CAT #HTB-22; MDA-MB-231 was obtained from ATCC CAT #HTB-26; MDA-MB-468 was obtained from ATCC CAT #HTB-132; RT112/84 was a gift of WuXI AppTec, obtained from EECACC, CODE #85061106; RT4 was obtained from ATCC CAT #HTB-2; CFPAC-1 was obtained from ATCC CAT #CRL-1918; NCI-H2110 was obtained from ATCC CAT #CRL-5924; JIMT-1 was obtained from Wuxi Apptech; Capan-1 was obtained from ATCC CAT #CRL-1573; antibody pertuzumab is prepared according to the known sequence; optimized recombinant enzyme Sortase A derived from Staphylococcus aureus is prepared in E. coli.

### Example 1 Construction of Antibody Expression Vector, Antibody Expression, Purification and Identification

### 1.1 Production of the Modified Anti-Human HER2 Antibody Ab0036

The expression plasmids for antibody Ab0036 (light chain SEQ ID NO: 9, heavy chain: SEQ ID NO: 10) were constructed as follows. The sequence of the antibody Ab0036: based on the amino acid sequence of Pertuzumab, and GALPETGG was introduced at the C-terminal of the light chain, wherein LPETGG is the recognition sequence of the ligase donor substrate, and GA is a spacer sequence. The plasmids were transfected into CHO cells and the cell population was established and screened for a highly expressed cell population, which was cultured with reference to the culture process of Pertuzumab in a 5-10 L reactor, and supernatant was collected.

### 1.2 the Purification of Antibody Ab0036

The purification of Ab0036 was carried out in a standard process using the combination of MabSelect affinity chromatography, the purified products were dissolved in the original Pertuzumab drug buffer (20 mM L-histidine acetate (pH 6.0), 120 mM sucrose and 0.02% polysorbate 20), and frozen in small aliquots.

### 1.3 the Quality Control of Antibody Ab0036

The purity of the above purified antibody Ab0036 is ≥95% by SDS-PAGE; the content of high molecular weight polymer of the sample is less than 5% by SEC-HPLC; endotoxin content is less than 1 EU/mg.

### 1.4 Preparation of Other Modified Anti-Human Antibodies

According to a similar method, a modified anti-human FGFR3 antibody Ab0206 (light chain SEQ ID NO: 19, heavy chain: SEQ ID NO: 20) is prepared. Ab0206 includes GALPETGG at the C-terminal of the light chain, wherein LPETGG is the recognition sequence of the ligase donor substrate, and GA is a spacer sequence.

According to a similar method, a modified anti-human HER2 antibody Ab0001 (light chain SEQ ID NO: 29, heavy chain: SEQ ID NO: 30) is prepared. Ab0001 includes GALPETGG at the C-terminal of the light chain, wherein LPETGG is the recognition sequence of the ligase donor substrate, and GA is a spacer sequence.

The specific sequences can be found in the attached Sequence Listing.

### Example 2-1 Preparation of Linker-Payload Intermediate EB302-2-4-1

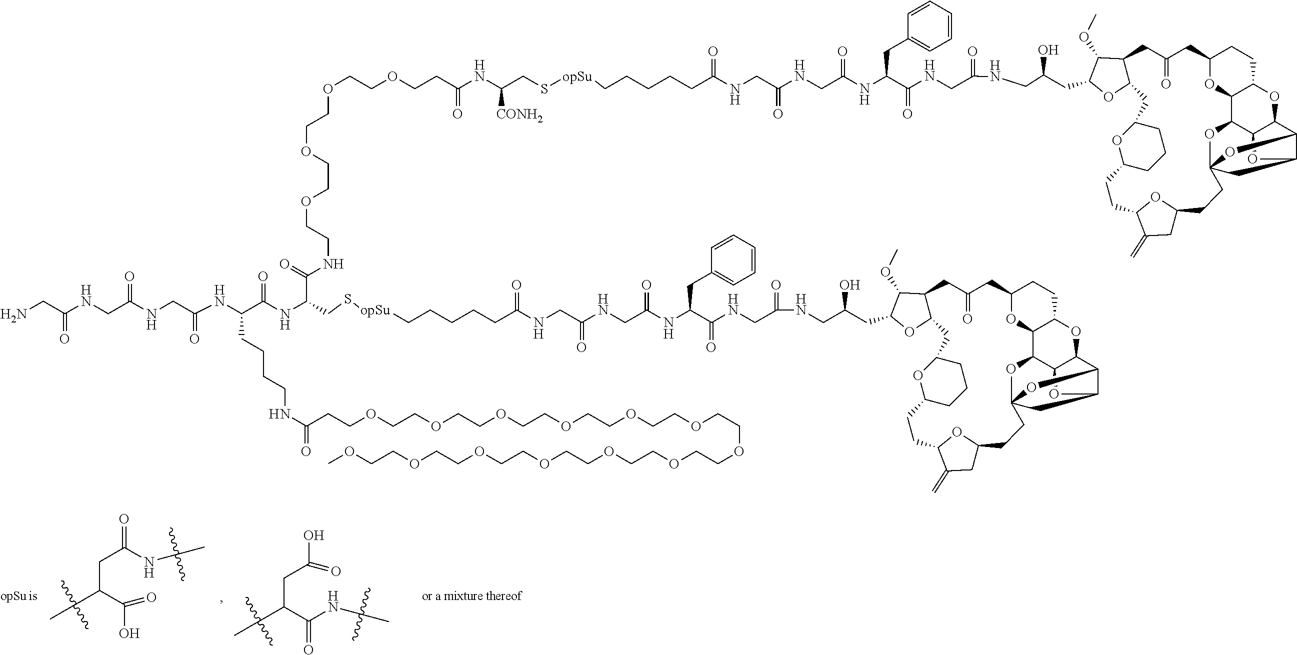

### 2-1.1 Preparation of Intermediate Mc-GGFG-Eribulin

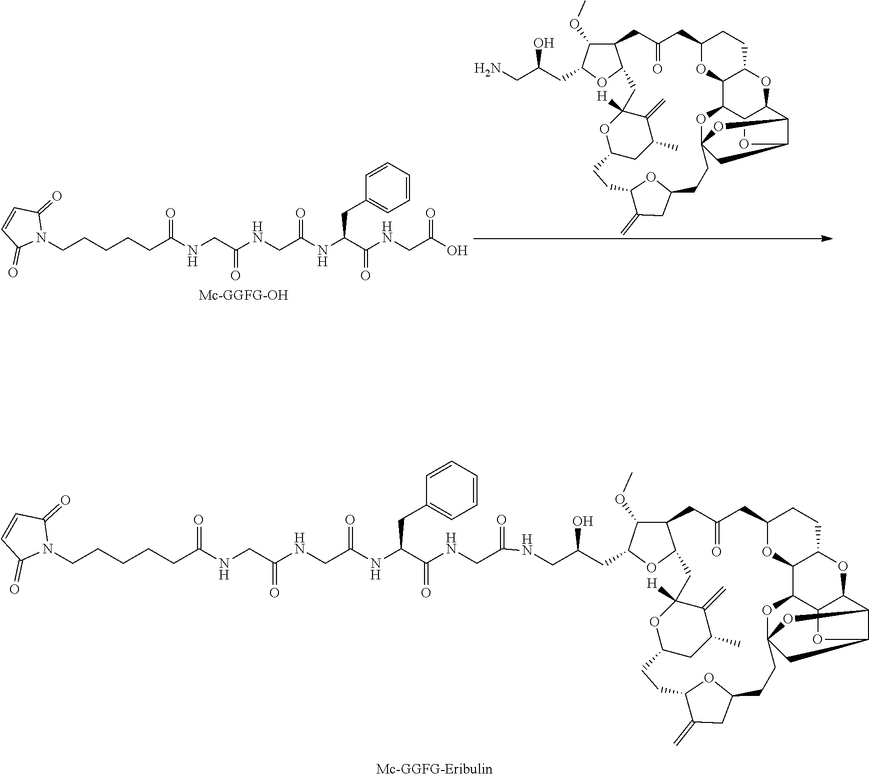 [SMILES: O=C(O)CNC(=O)[C@H](Cc1ccccc1)NC(=O)CNC(=O)CNC(=O)CCCCCN1C(=O)C=CC1=O ; C=C1C[C@@H]2CC[C@@]34C[C@H]5OC6[C@@H](O[C@H]7CC[C@H](CC(=O)C[C@@H]8[C@@H](OC)[C@@H](C[C@H](O)CN)O[C@H]8C[C@H]8O[C@@H](CC[C@@H]1O2)C[C@@H](C)C8=C)O[C@@H]7[C@@H]6O3)[C@H]5O4 ; C=C1C[C@@H]2CC[C@@]34C[C@H]5OC6[C@@H](O[C@H]7CC[C@H](CC(=O)C[C@@H]8[C@@H](OC)[C@@H](C[C@H](O)CNC(=O)CNC(=O)[C@H](Cc9ccccc9)NC(=O)CNC(=O)CNC(=O)CCCCCN9C(=O)C=CC9=O)O[C@H]8C[C@H]8O[C@@H](CC[C@@H]1O2)C[C@@H](C)C8=C)O[C@@H]7[C@@H]6O3)[C@H]5O4]

The compound Mc-GGFG-OH is commercially available.

The compound Mc-GGFG-OH (35 mg), Eribulin (50 mg) and DIPEA (45 mg) were dissolved in DMF (1 mL), and HATU (35 mg) was added to the solution. The reaction was stirred at room temperature for 2 h. After the reaction was completed, the reaction mixture was subjected to prep-HPLC (Mobile phase: A: water (10 mM ammonium acetate), B: acetonitrile; gradient elution), and the eluent was then lyophilized to give Mc-GGFG-Eribulin (33 mg, 44% yield). Calcd [M+H]⁺: 1241.6, measured: [M+H]⁺=1241.6. This compound is used to prepare the Linker-Payload intermediate (formula (IV) compound), and is also used to directly connect to the (optionally modified) antibody to prepare reference ADCs.

### 2-1.2 Preparation of Linker HX

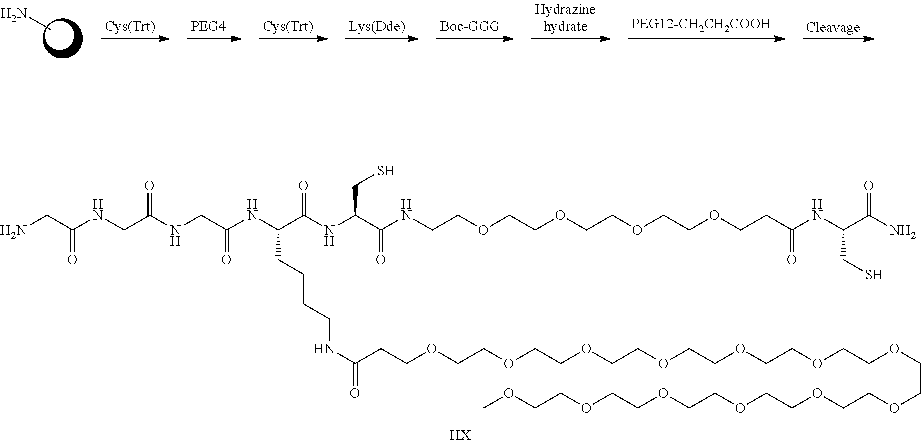

The linker HX synthesized by a conventional solid phase polypeptide synthesis using Rink-amide-MBHA-resin. Fmoc was used to protect the amino acid in the linker. The conjugation reagent was selected from HOBT, HOAt/DIC, DCC, EDCI or HATU. After synthesis, the resin was cleaved using trifluoroacetic acid. The product was purified by HPLC, lyophilized and stored for use. Theoretical Mass: 1383.70, measured: [M−H]⁻=1382.6.

### 2-1.3 Preparation of Linker-Payload Intermediate EB302-2-4-1

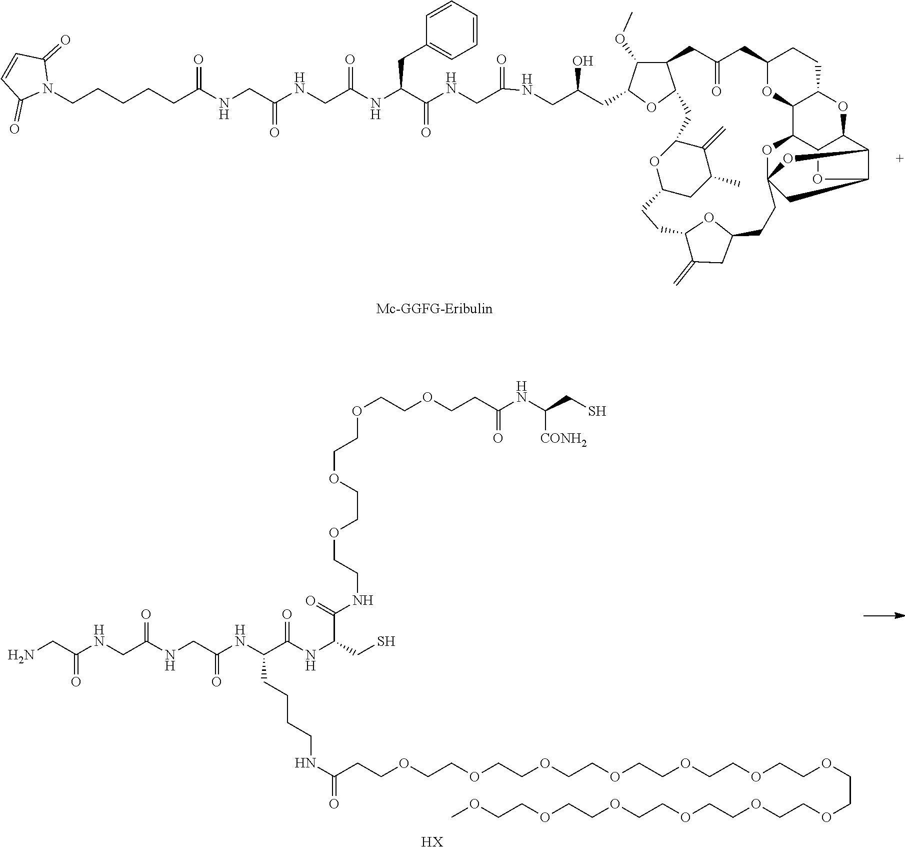

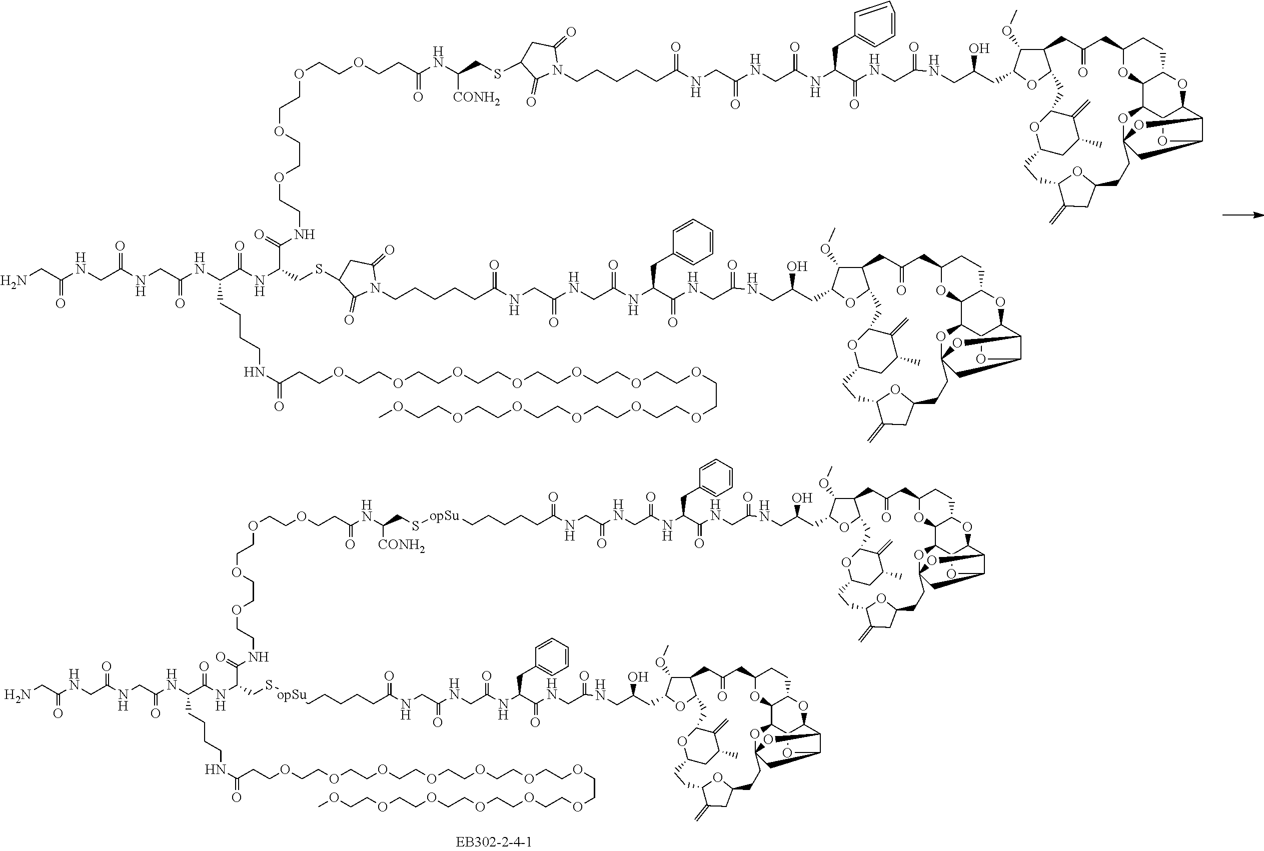

The linker HX (20 mg) and intermediate Mc-GGFG-Eribulin (33 mg) were dissolved in water (0.5 mL) and DMF (0.5 mL), respectively, and then thoroughly mixed to give a mixture, which was reacted at room temperature for 16 h. Once the reaction was completed, the reaction mixture was analyzed by LCMS (Calcd [(M+3H+H₂O)/3]⁺: 1295.3, measured: [(M+3H+H₂O)/3]⁺=1295.4). The reaction mixture was cooled to 0° C. with an ice bath, and 0.1% lithium hydroxide aqueous solution was added slowly dropwise. After the pH of the reaction system was adjusted to 12-13, and kept at 0° C. for 0.5 h. After the raw materials disappeared, the reaction mixture was neutralized with 30% acetic acid aqueous solution, and purified by pre-HPLC (mobile phase: A: water (10 mM ammonium acetate), B: acetonitrile; gradient elution) and lyophilized to obtain 21 mg of linker-payload compound EB302-2-4-1. Two-step yield is 40%. Calcd [(M+3H+H₂O)/3]⁺: 1307.3, measured: [(M+3H+H₂O)/3]⁺=1307.3.

### Example 2-2 Preparation of EB301-2-1-1

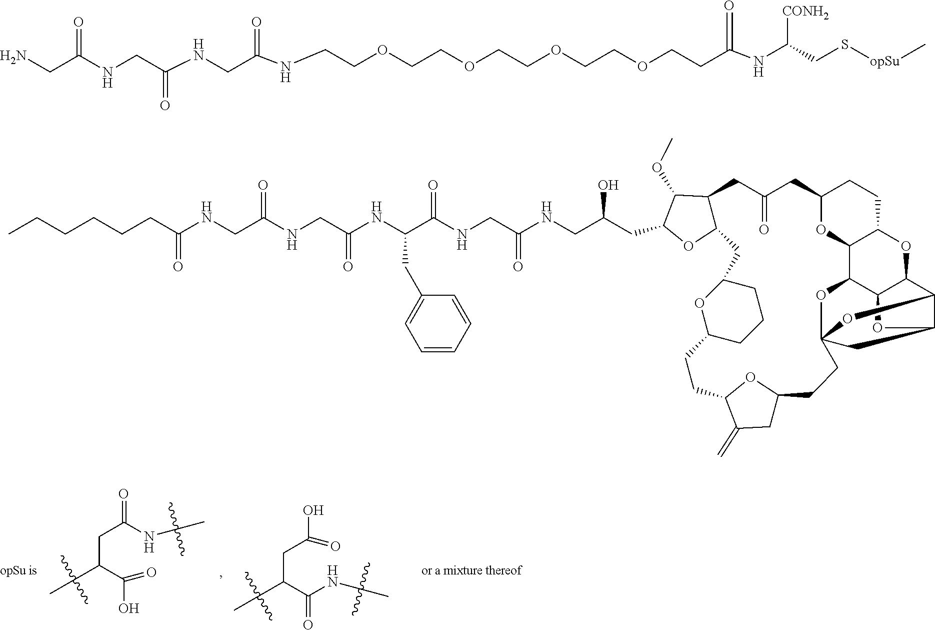

### Preparation Procedure

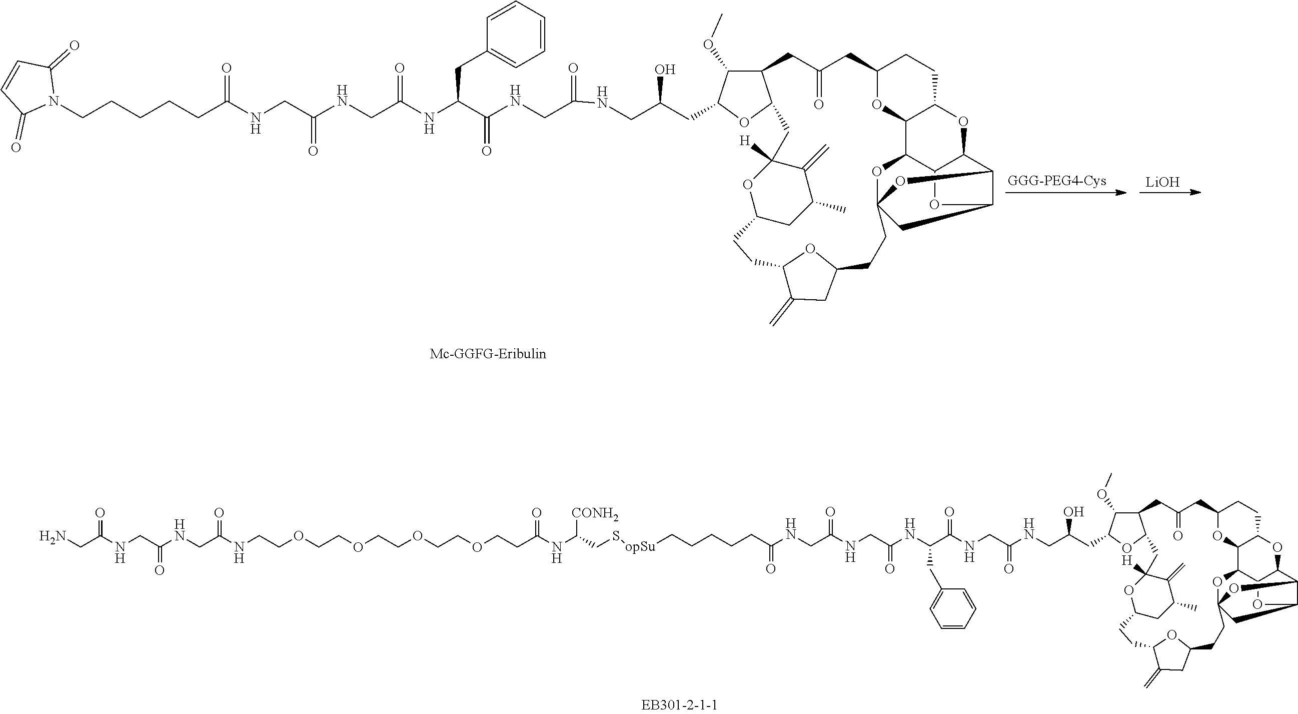

A solution of 20 mg Mc-GGFG-Eribulin (0.016 mmol) and 10.4 mg GGG-PEG4-Cys-NH₂ (0.019 mmol) in 1 mL DMF-H₂O (DMF:H₂O=1:1) was stirred at room temperature for 16 h. The reaction was monitored by HPLC. After the reaction was complete, the reaction solution was cooled to 0-5° C., and 0.1% LiOH solution was added dropwise. The pH of the reaction solution was adjusted to 12-13. The reaction solution was allowed to warm to room temperature and then stirred for 30 min, and the reaction was monitored by HPLC until the reaction was complete. The reaction solution was neutralized with 10% acetic acid, and then purified by prep-HPLC. The fractions were pooled for lyophilization, and thus afforded compound EB301-2-1-1 as a white solid (16.2 mg, yield 56%). MS (ESI) Calcd for C₈₅H₁₃₀N₁₂O₂₈S [(M+2H)/2]⁺: 899.5, found: 900.0.

### Example 2-3 Preparation of EB301-2-101-1

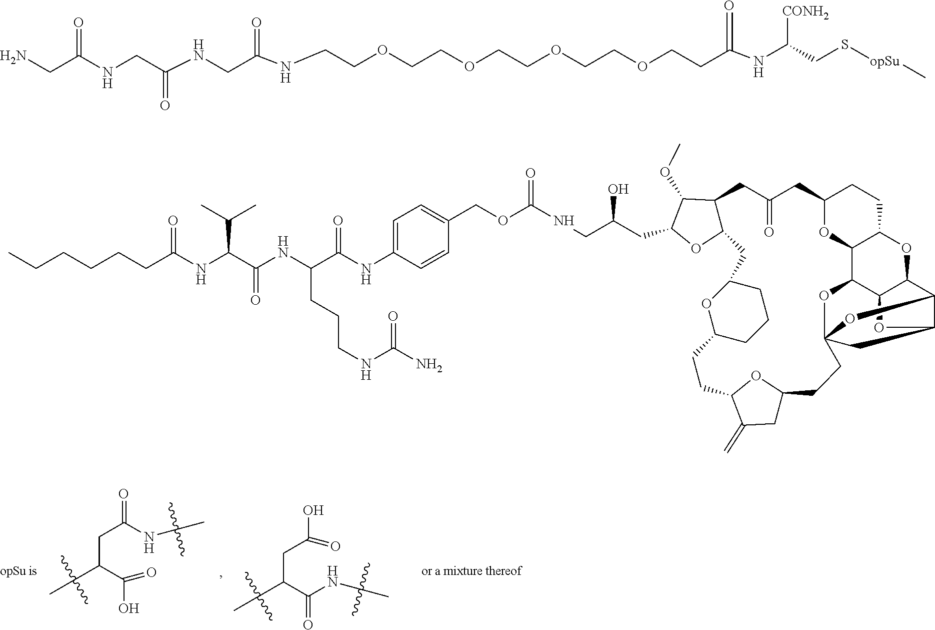

### 2-3.1 Preparation of Intermediate Mc-Val-Cit-PABC-Eribulin

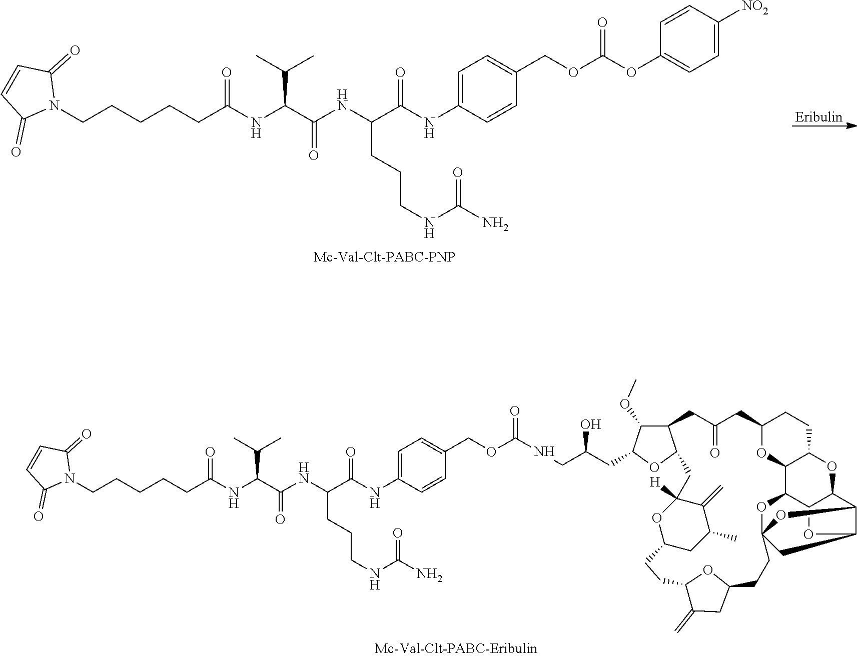 [SMILES: CC(C)[C@H](NC(=O)CCCCCN1C(=O)C=CC1=O)C(=O)NC(CCCNC(N)=O)C(=O)Nc1ccc(COC(=O)Oc2ccc([N+](=O)[O-])cc2)cc1 ; C=C1C[C@@H]2CC[C@@]34C[C@H]5OC6[C@@H](O[C@H]7CC[C@H](CC(=O)C[C@@H]8[C@@H](OC)[C@@H](C[C@H](O)CNC(=O)OCc9ccc(NC(=O)C(CCCNC(N)=O)NC(=O)[C@@H](NC(=O)CCCCCN%10C(=O)C=CC%10=O)C(C)C)cc9)O[C@H]8C[C@H]8O[C@@H](CC[C@@H]1O2)C[C@@H](C)C8=C)O[C@@H]7[C@@H]6O3)[C@H]5O4]

To a solution of 20 mg Eribulin (0.024 mmol) in 1 mL DMF was added 6.2 mg DIPEA (0.048 mmol). Then 17.8 mg Mc-Val-Cit-PABC-PNP was added in two portions. The reaction solution was stirred at room temperature for 2 h. The reaction was monitored by HPLC. After the reaction was complete, the reaction solution was purified by prep-HPLC. The fractions were pooled for lyophilization, and thus afforded compound Mc-Val-Cit-PABC-Eribulin as a white solid (25 mg, yield 78%). MS (ESI) Calcd for C₆₉H₉₉N₇O₁₉ [M+H]⁺: 1328.6, found: 1328.3.

### 2-3.2 Preparation of EB301-2-101-1

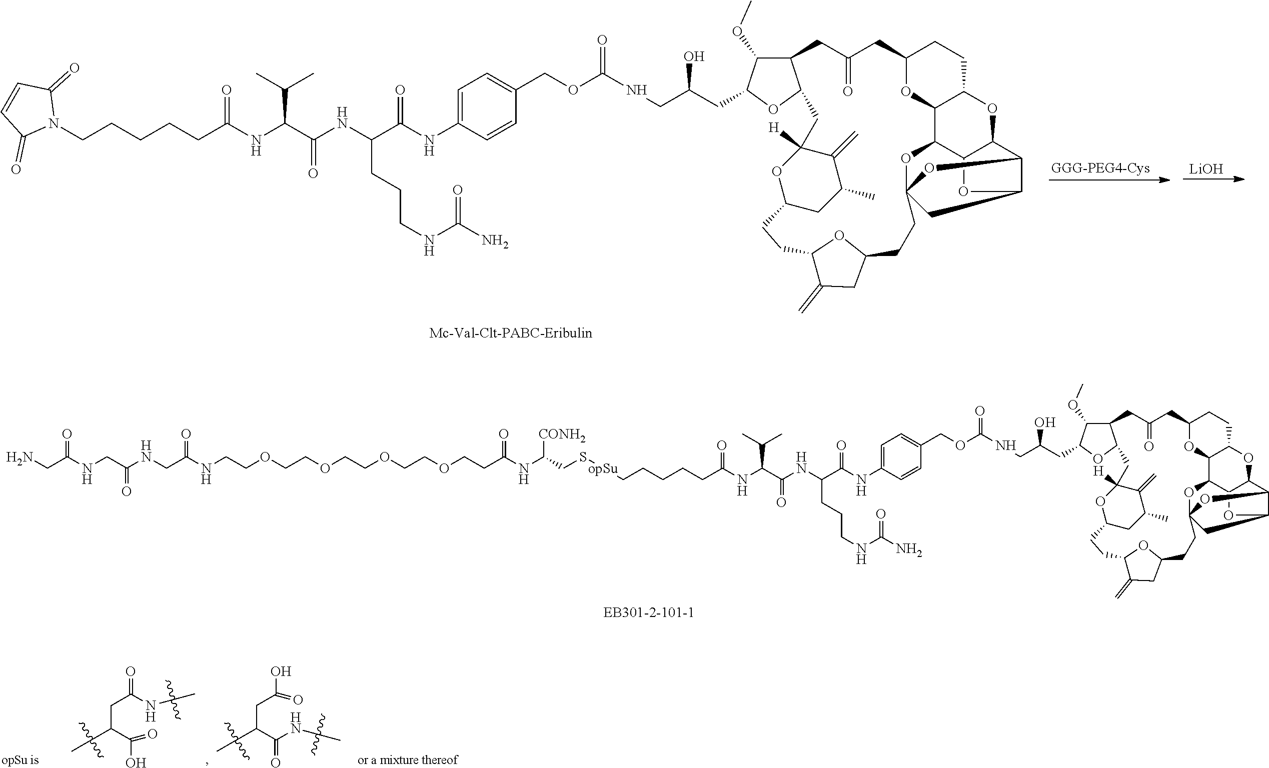

A solution of 17.7 mg of Mc-Val-Cit-PABC-Eribulin (0.013 mmol) and 10.8 mg of GGG-PEG4-Cys-NH₂ (0.02 mmol) in 1 mL DMF-H₂O (DMF:H₂O=1:1) was stirred at room temperature for 16 h. The reaction was monitored by HPLC. After the reaction was complete, the reaction solution was cooled to 0-5° C., and 0.1% LiOH solution was added dropwise. The pH of the reaction solution was adjusted to 12-13. The reaction solution was allowed to warm to room temperature and then stirred for 30 min, and the reaction was monitored by HPLC until the reaction was complete. The reaction solution was neutralized with 10% acetic acid, and then purified by prep-HPLC. The fractions were pooled for lyophilization, and thus afforded compound EB301-2-101-1 as a white solid (18 mg, yield 75%). MS (ESI) Calcd for C₈₉H₁₃₈N₁₃O₂₉S [M+H]⁺: 1884.9, found: 1885.2.

### Example 2-3 Preparation of EB302-2-104-1

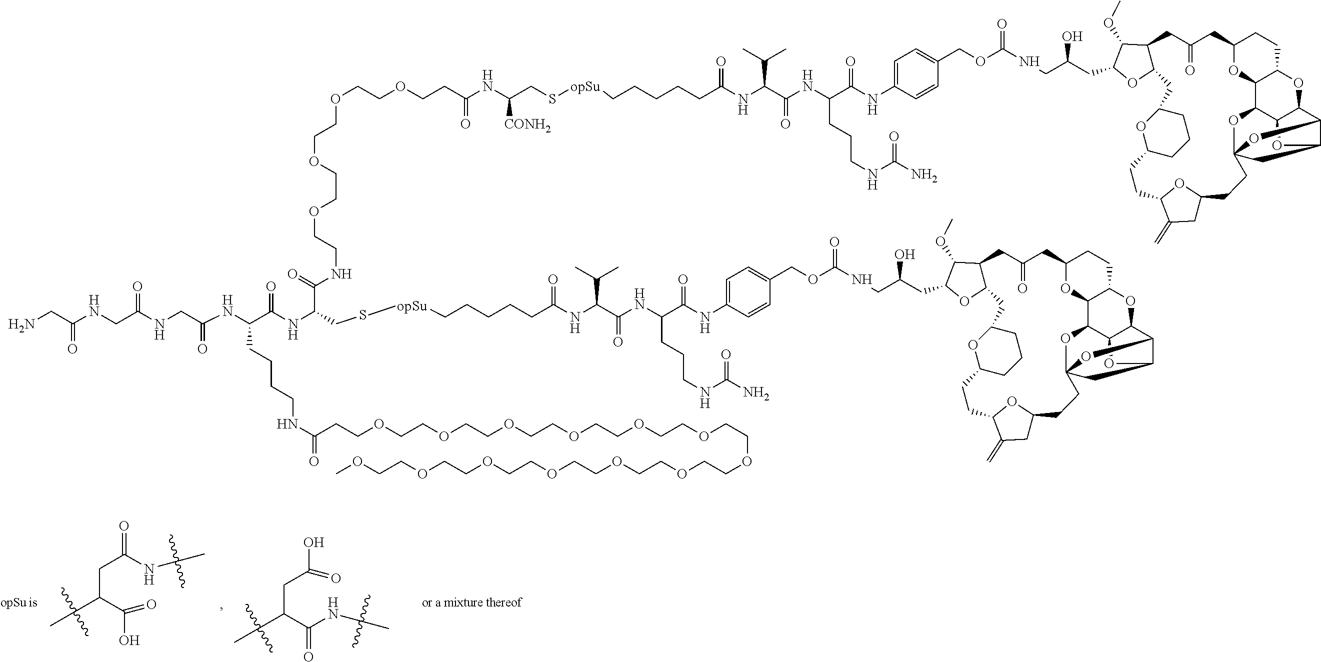

### Preparation Procedure

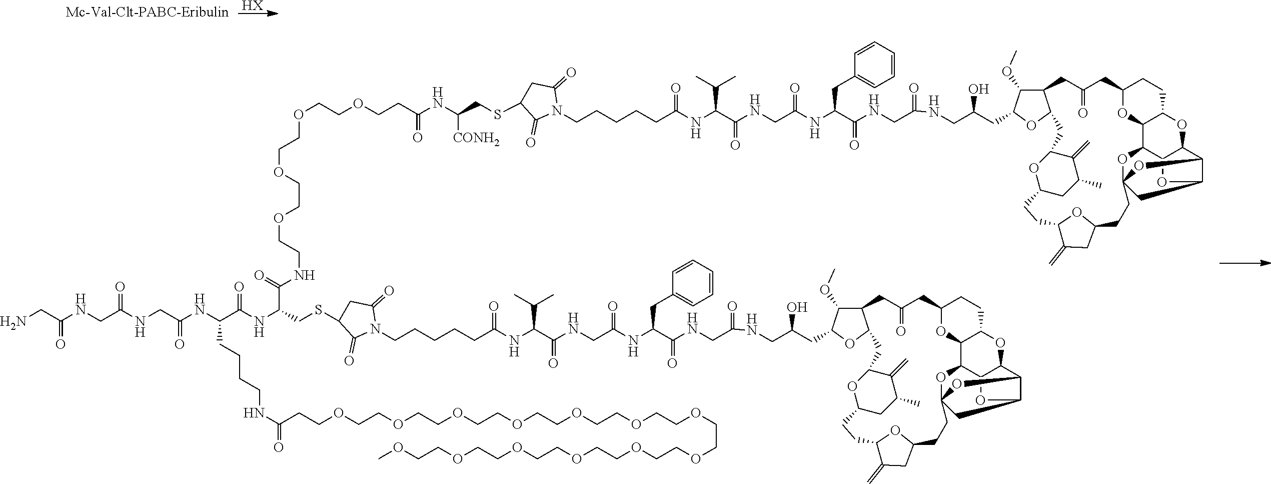

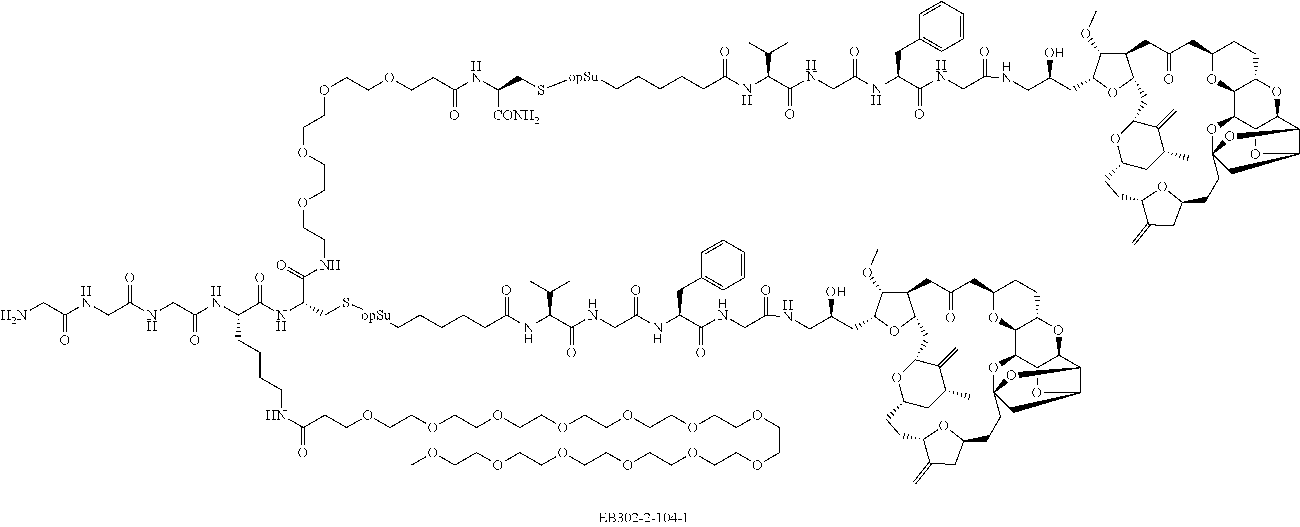

A solution of 18.1 mg Mc-Val-Cit-PABC-Eribulin (0.0136 mmol) and 10.5 mg linker HX (0.007 mmol, structure is depicted above) in 1 mL DMF-H₂O (DMF:H₂O=1:1) was stirred at room temperature for 16 h. The reaction was monitored by HPLC. After Mc-Val-Cit-PABC-Eribulin completely disappeared, the reaction solution was cooled to 0-5° C., and 0.1% LiOH solution was added dropwise. The pH of the reaction solution was adjusted to 12-13. The reaction solution was allowed to warm to room temperature and then stirred for 30 min, and the reaction was monitored by HPLC until the reaction was complete. The reaction solution was neutralized with 10% acetic acid, and then purified by prep-HPLC. The fractions were pooled for lyophilization, and thus afforded compound EB302-2-104-1 as a white solid (9.4 mg, yield 31%). MS (ESI) Calcd for C₁₉₄H₁₄₃N₁₄O₂₉S [(M+2H+NH₄)/3]⁺: 1361.0, found: 1360.2.

### Example 3 Preparation of Targeting Molecule-Pharmaceutical Conjugates

### 3.1 Preparation of ADCs

The Linker-payload intermediate EB302-2-4-1, EB301-2-1-1, EB301-2-101-1, EB302-2-104-1, were conjugated to an antibody in a site-specific manner by a ligase to form ADCs. The method for conjugation reaction can be found in WO2015165413A1. The resulting ADCs are as listed in the following Table 3.

TABLE 3
Prepared ADCs

| Name of ADC | Linker-Payload | Antibody |
|---|---|---|
| EC302-2-4-1-1 | EB302-2-4-1 | Ab0036 |
| EC302-2-4-1-2 | EB302-2-4-1 | Ab0206 |
| EC301-2-1-1-1 | EB301-2-1-1 | Ab0036 |
| EC302-2-4-1-3 | EB302-2-4-1 | Ab0001 |
| EC301-2-1-1-3 | EB301-2-1-1 | Ab0001 |
| EC301-2-101-1-3 | EB301-2-101-1 | Ab0001 |
| EC302-2-104-1-3 | EB302-2-104-1 | Ab0001 |

Note:
In preparation process of ADC, the upstream peptide bond of GG in the LPETGG sequence in the antibody is cleaved by Sortase A, and the resulting intermediate is linked to the free N-terminal of G₃ in linker-payload to generate a new peptide bond, thereby forming the resulting amino acid sequence LPXTG₃.

### 3.2 HIC-HPLC Detection and Analysis

The DAR distribution of the conjugates prepared in above example 3.1 was analyzed by HIC-HPLC. The antibody Ab0036 without cytotoxin was less than 2%; the coupled product mainly contains EC302-2-4-1-1 with DAR of 4, and the DAR of EC302-2-4-1-1 was calculated to be about 3.44. The antibody Ab0206 without cytotoxin was less than 2%; the coupled product mainly contains EC302-2-4-1-2 with DAR of 4, and the DAR of EC302-2-4-1-2 was calculated to be about 3.35. DAR values of EC301-2-1-1-1, EC301-2-1-1-3, EC302-2-4-1-3, EC301-2-101-1-3, EC302-2-104-1-3 were calculated to be about 1.88, about 1.81 about 3.44, about 1.72, about 2.94, respectively.

### 3.3 SEC-HPLC Detection and Analysis for EC302-2-4-1-1 and EC302-2-4-1-2

The degree of high molecular weight aggregation of the conjugates prepared in above 3.1 was analyzed by SEC-HPLC. The conjugates EC302-2-4-1-1 and EC302-2-4-1-2 each has a purity of 98.78% and 94.99%, respectively. No high molecular weight polymer was detected in the conjugates, indicating that the coupling reaction conditions were mild and did not cause damage to the antibody structure.

The free payload eribulin was not detected in the samples of the conjugates EC302-2-4-1-1 and EC302-2-4-1-2.

### Effect Example 1 Effect of Conjugates Targeting HER2 on Cell Proliferation

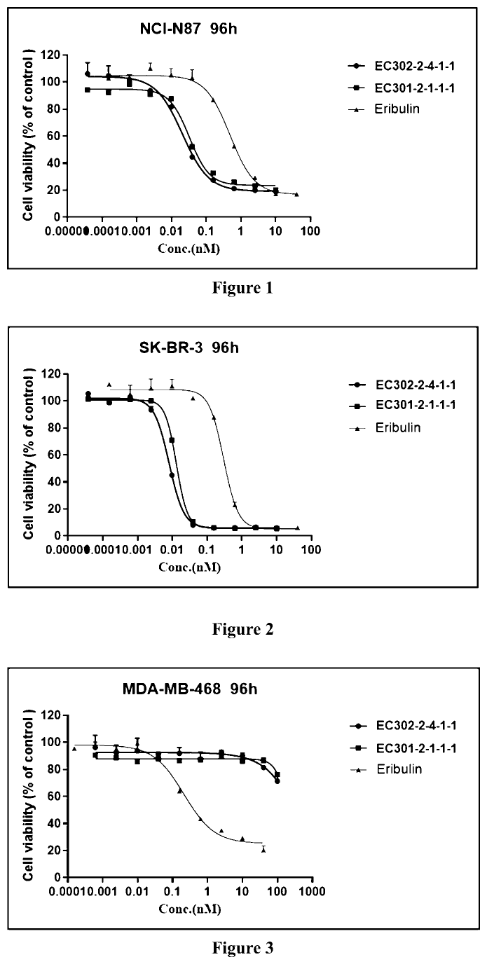

Cytotoxicity assays were performed using HER2 high-expressing cancer cells NCI-N87 (FIG. 1) and SK-BR-3 (FIG. 2), as well as a HER2 negative cell line MDA-MB-468 (FIG. 3) to analyze the effect of conjugates on tumor cell proliferation. The tested articles included conjugates EC302-2-4-1-1 and EC301-2-1-1-1, and a small molecule eribulin (structure is depicted above). In brief, 5000 cells were plated in 96-well plates, and cells were able to attach overnight. Cells were treated with indicated drugs with various concentrations for 96 h. Cell viabilities were examined by CellTiter-Glo® Luminescent Cell Viability Assay, and percentage of cell viability was calculated.

All the NCI-N87, SK-BR-3 and MDA-MB-468 cell lines were sensitive to Eribulin. In HER2 high-expressing NCI-N87 and SK-BR-3, all drugs showed good cell proliferation-inhibitory effects superior than cribulin. EC302-2-4-1-1 was more potent than EC301-2-1-1-1. This difference of potency may be caused by the different number of the payload. No obvious cytotoxicity was observed in HER2 negative cells, demonstrating the stability of the ADCs, with reduced off-target release of the payload and thereby good safety. These indicate that EC302-2-4-1-1 and EC301-2-1-1-1 exert HER2-dependent cytotoxic activity in a dose dependent manner, exhibiting good targeting ability, outstanding efficacy and good safety.

TABLE 4
IC₅₀ values according to cell proliferation-inhibitory tests

IC₅₀ (nM)

| Sample | N87 | SK-BR-3 | MDA-MB-468 |
|---|---|---|---|
| EC302-2-4-1-1 | 0.02034 | 0.008051 | N/A |
| EC301-2-1-1-1 | 0.03245 | 0.01304 | N/A |
| Eribulin | 0.4479 | 0.2980 | 0.2106 |

### Effect Example 2 Effect of Conjugates Targeting HER2 on Cell Proliferation

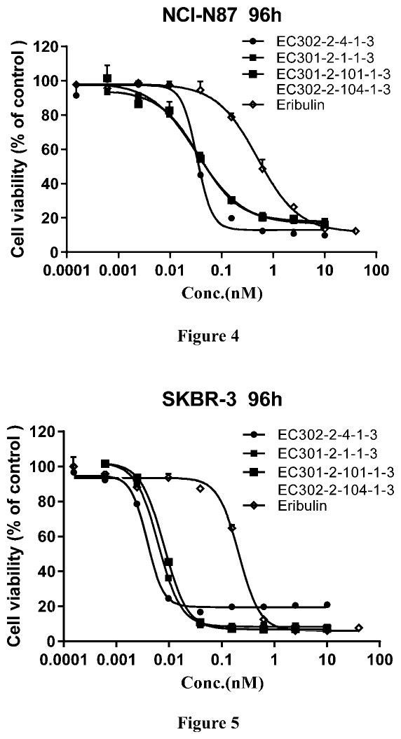

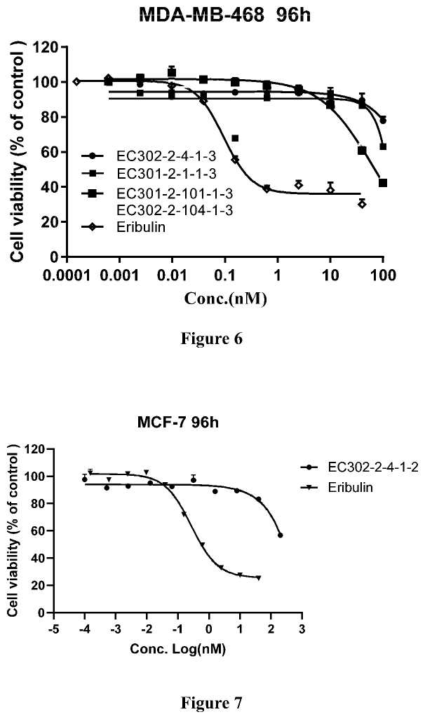

Cytotoxicity assays were performed using HER2 high-expressing cancer cells NCI-N87 (FIG. 4) and SK-BR-3 (FIG. 5), as well as a HER2 negative cell line MDA-MB-468 (FIG. 6) to analyze the effect of conjugates on tumor cell proliferation. The tested articles included conjugates EC302-2-4-1-3, EC301-2-1-1-3, EC301-2-101-1-3 and EC302-2-104-1-3, and the small molecule eribulin (structure is depicted above). In brief, 5000 cells were plated in 96-well plates, and cells were able to attach overnight. Cells were treated with indicated drugs with various concentrations for 96 h. Cell viabilities were examined by CellTiter-Glo® Luminescent Cell Viability Assay, and percentage of cell viability was calculated.

In HER2 high-expressing NCI-N87 and SK-BR-3, all drugs showed good cell proliferation-inhibitory effects superior than eribulin. The cell proliferation-inhibitory effects of EC302-2-104-1-3 and EC301-2-101-1-3 were similar. EC302-2-104-1-3 was more potent than EC301-2-101-1-3. This difference of potency may be caused by different numbers of the payload attached. The cell proliferation-inhibitory effects of EC301-2-1-1-3 and EC301-2-101-1-3 were similar. No obvious cytotoxicity was observed in HER2 negative cells, demonstrating the stability of the ADCs, with reduced off-target release of the payload and thereby good safety.

TABLE 5
IC₅₀ values according to cell proliferation-inhibitory tests

IC₅₀ (nM)

| Sample | N87 | SK-BR-3 | MDA-MB-468 |
|---|---|---|---|
| EC302-2-4-1-3 | 0.0333 | 0.003793 | NA |
| EC301-2-1-1-3 | 0.0372 | 0.006292 | NA |
| EC301-2-101-1-3 | 0.03351 | 0.007985 | NA |
| EC302-2-104-1-3 | 0.02470 | 0.00439 | NA |
| Eribulin | 0.5013 | 0.2114 | 0.09399 |

### Effect Example 3 Effect of Conjugates Targeting FGFR3 on Cell Proliferation

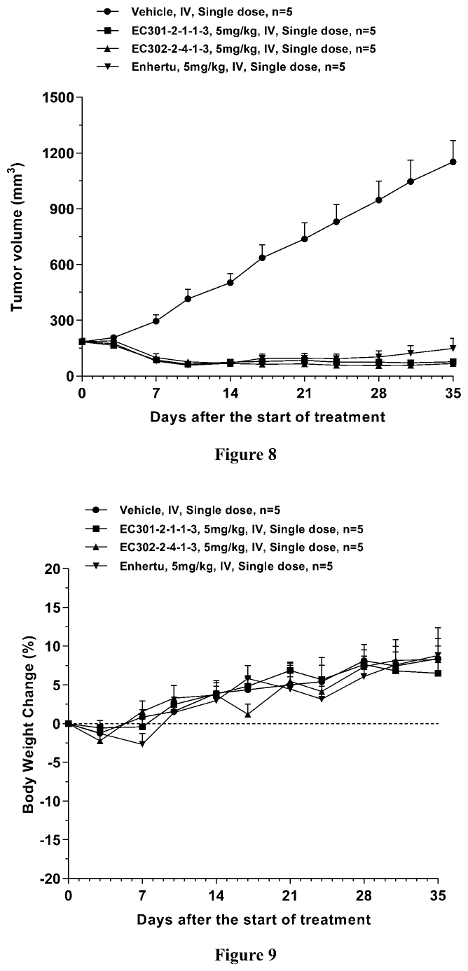

Cytotoxicity assays were performed using FGFR3 high-expressing cancer cells RT112/84 and RT4, FGFR3 negative cancer cell MCF-7 (FIG. 7) to analyze the effect of conjugates on tumor cell proliferation. The tested articles included conjugate EC302-2-4-1-2, and a small molecule eribulin (structure is depicted above). In brief, 2000 cells (RT112/84) or 5000 cells (RT4 or MCF-7) were plated in 96-well plates, and cells were able to attach overnight. Cells were treated with indicated drugs with various concentrations for 96 h. Cell viabilities were examined by CellTiter-Glo® Luminescent Cell Viability Assay, and percentage of cell viability was calculated.

All the RT112/84, RT4 and MCF-7 cell lines were sensitive to eribulin. EC302-2-4-1-2 inhibited the cell proliferation of only FGFR3-positive RT112/84 and RT4 cells. No cytotoxicity was observed in FGFR3 negative cell MCF-7, demonstrating the stability of EC302-2-4-1-2, with reduced off-target release of the payload and thereby good safety.

### Effect Example 4 In Vivo Efficacy in NCI-N87 CDX Model

NCI-N87 human gastric cancer cells were implanted into the right flank of female BALB/c nude mice. The animals were randomly divided into 4 groups with 5 tumor-bearing mice in each group on day 6 after tumor inoculation. Meanwhile, on the day of grouping, tumor-bearing mice were treated with 5 mg/kg dose of EC301-2-1-1-3, EC302-2-4-1-3 or Enhertu respectively. Tumor volume was measured twice weekly in two dimensions using a caliper, and the volume was expressed in mm³ using the formula: V=0.5 a×b² where a and b are the long and short diameters of the tumor, respectively. The body weight of the mice were measured twice a week, and abnormal behavior was observed daily such as mobility, food and water consumption.

The T/C (%) value is calculated for each group using the formula: T/C %=TRTV/CRTV×100% (TRTV: treatment group RTV; CRTV: control group RTV; RTV: relative tumor volume). The RTV=Vt/V0, V0 is the average tumor volume on the first day of treatment, VT is the average tumor volume on a given day, the data of TRTV is from the same day as CRTV.

TGI is calculated for each group using the formula: TGI (%)=[1−(Ti−T0)/(Vi−V0)]×100; Ti is the average tumor volume of a treatment group on a given day, T0 is the average tumor volume of the treatment group on the first day of treatment, Vi is the average tumor volume of the vehicle control group on the same day with Ti, and V0 is the average tumor volume of the vehicle group on the first day of treatment.

The results on tumor volumes in different treatment groups at different time points after treatment start are shown in the Table 6, FIG. 8 and FIG. 9. In this model, EC301-2-1-1-3, EC302-2-4-1-3 and Enhertu all showed excellent tumor inhibition effect. And EC301-2-1-1-3 and EC302-2-4-1-3 at the same concentration had slightly better tumor inhibition than Enhertu, despite having half the DAR of Enhertu. Animal body weight was monitored regularly as an indirect indication of toxicity. None of the treatment groups showed significant average body weight loss.

TABLE 6
Tumor growth inhibition in the NCI-N87 xenograft model
calculated based on tumor volume measurements at PG-D35

| | | Tumor volume | | | |
|---|---|---|---|---|---|
| | | (mm³) | T/C | TGI | |
| Treatment | N | on day 35 | (%) | (%) | p value |
| Vehicle | 5 | 1,153 ± 114 | — | — | — |
| EC301-2-1-1-3 | 5 | 76 ± 13 | 6.63 | 111.13 | 0.004 |
| EC302-2-4-1-3 | 5 | 68 ± 12 | 5.89 | 112.01 | 0.004 |
| Enhertu | 5 | 148 ± 55 | 12.81 | 103.79 | 0.002 |

### Effect Example 5 In Vivo Efficacy in Capan-1 CDX Model

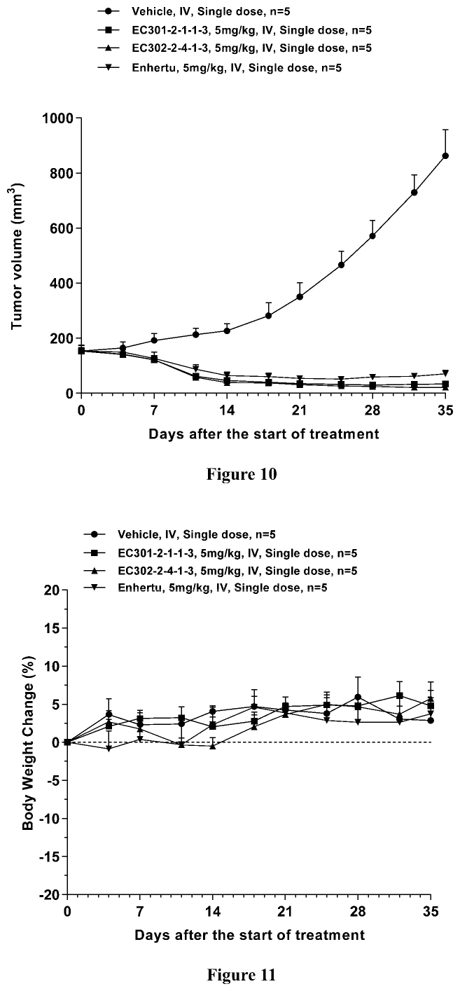

Capan-1 CDX model refers to NCI-N87 CDX model in vivo efficacy evaluation experimental method. The results on tumor volumes in different treatment groups at different time points after treatment start are shown in the Table 7, FIG. 10 and FIG. 11. In the Capan-1 CDX model, EC301-2-1-1-3, EC302-2-4-1-3 and Enhertu all showed obviously tumor inhibition effect. And EC301-2-1-1-3 and EC302-2-4-1-3 at the same concentration had slightly better tumor inhibition than Enhertu, despite having half the DAR of Enhertu. Each drug was well tolerated in all groups. None of the treatment groups showed significant average body weight loss.

TABLE 7
Tumor growth inhibition in the Capan-1 xenograft model
calculated based on tumor volume measurements on PG-D35

| | | Tumor volume | | | |
|---|---|---|---|---|---|
| | | (mm³) | T/C | TGI | |
| Treatment | N | on day 35 | (%) | (%) | p value |
| Vehicle | 5 | 862 ± 95 | — | — | — |
| EC301-2-1-1-3 | 5 | 33 ± 6 | 3.87 | 116.83 | 0.005 |
| EC302-2-4-1-3 | 5 | 22 ± 2 | 2.53 | 118.54 | 0.005 |
| Enhertu | 5 | 71 ± 11 | 8.23 | 111.58 | 0.006 |

Sequence Listing

SEQ ID NO: 1: Ab0036 HCDR1
DYTMD
SEQ ID NO: 2: Ab0036 HCDR2
DVNPNSGGSIYNQRFKG
SEQ ID NO: 3: Ab0036 HCDR3
NLGPSFYFDY
SEQ ID NO: 4: Ab0036 LCDR1
KASQDVSIGVA
SEQ ID NO: 5: Ab0036 LCDR2
SASYRYT
SEQ ID NO: 6: Ab0036 LCDR3
QQYYIYPYT
SEQ ID NO: 7: Ab0036 Light chain variable region:
DIQMTQSPSSLSASVGDRVTITCKASQDVSIGVAWYQQKPGKAPKLLIYSASYRYTGV
PSRFSGSGSGTDFTLTISSLQPEDFATYYCQQYYIYPYTFGQGTKVEIKRTV
SEQ ID NO: 8: Ab0036 Heavy chain variable region:
EVQLVESGGGLVQPGGSLRLSCAASGFTFTDYTMDWVRQAPGKGLEWVADVNPNSG
GSIYNQRFKGRFTLSVDRSKNTLYLQMNSLRAEDTAVYYCARNLGPSFYFDYWGQGT
LVTVSS
SEQ ID NO: 9: Ab0036 Light chain full length:
DIQMTQSPSSLSASVGDRVTITCKASQDVSIGVAWYQQKPGKAPKLLIYSASYRYTGV
PSRFSGSGSGTDFTLTISSLQPEDFATYYCQQYYIYPYTFGQGTKVEIKRTVAAPSVFIFP
PSDEQLKSGTASVVCLLNNFYPREAKVQWKVDNALQSGNSQESVTEQDSKDSTYSLS
STLTLSKADYEKHKVYACEVTHQGLSSPVTKSFNRGECGALPETGG
SEQ ID NO: 10: Ab0036 Heavy chain full length:
EVQLVESGGGLVQPGGSLRLSCAASGFTFTDYTMDWVRQAPGKGLEWVADVNPNSG
GSIYNQRFKGRFTLSVDRSKNTLYLQMNSLRAEDTAVYYCARNLGPSFYFDYWGQGT
LVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 11: Ab0206 HCDR1
SYGIS
SEQ ID NO: 12: Ab0206 HCDR2
WVSTYNGDTNYAQKFQG
SEQ ID NO: 13: Ab0206 HCDR3
VLGYYDSIDGYYYGMDV
SEQ ID NO: 14: Ab0206 LCDR1
GGNNIGDKSVH
SEQ ID NO: 15: Ab0206 LCDR2
LDTERPS
SEQ ID NO: 16: Ab0206 LCDR3
QVWDSGSDHVV
SEQ ID NO: 17: Ab0206 Light chain variable region:
QSVLTQPPSLSVAPGKTATFTCGGNNIGDKSVHWYRQKPGQAPVLVMYLDTERPSGIP
ERMSGSNFGNTATLTITRVEAEDEADYYCQVWDSGSDHVVFGGGTKLTVLGQP
SEQ ID NO: 18: Ab0206 Heavy chain variable region:
EVQLVQSGAEVKKPGASVKVSCKASGYMFTSYGISWVRQAPGQGLEWMGWVSTYN
GDTNYAQKFQGRVTVTTDTSTSTAYMELRSLRSEDTAVYYCARVLGYYDSIDGYYYG
MDVWGQGTTVTVSS
SEQ ID NO: 19: Ab0206 Light chain full length:
QSVLTQPPSLSVAPGKTATFTCGGNNIGDKSVHWYRQKPGQAPVLVMYLDTERPSGIP
ERMSGSNFGNTATLTITRVEAEDEADYYCQVWDSGSDHVVFGGGTKLTVLGQPKAAP
SVTLFPPSSEELQANKATLVCLISDFYPGAVTVAWKADSSPVKAGVETTTPSKQSNNK
YAASSYLSLTPEQWKSHRSYSCQVTHEGSTVEKTVAPAECSGALPETGG
SEQ ID NO: 20: Ab0206 Heavy chain full length:
EVQLVQSGAEVKKPGASVKVSCKASGYMFTSYGISWVRQAPGQGLEWMGWVSTYN
GDTNYAQKFQGRVTVTTDTSTSTAYMELRSLRSEDTAVYYCARVLGYYDSIDGYYYG
MDVWGQGTTVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRVEPKS
CDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 21: Ab0001 HCDR1
DTYIH
SEQ ID NO: 22: Ab0001 HCDR2
RIYPTNGYTRYADSVKG
SEQ ID NO: 23: Ab0001 HCDR3
WGGDGFYAMDY
SEQ ID NO: 24: Ab0001 LCDR1
RASQDVNTAVA
SEQ ID NO: 25: Ab0001 LCDR2
SASFLYS
SEQ ID NO: 26: Ab0001 LCDR3
QQHYTTPPT
SEQ ID NO: 27: Ab0001 Light chain variable region:
DIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIKRTV
SEQ ID NO: 28: Ab0001 Heavy chain variable region:
EVQLVESGGGLVQPGGSLRLSCAASGFNIKDTYIHWVRQAPGKGLEWVARIYPTNGY
TRYADSVKGRFTISADTSKNTAYLQMNSLRAEDTAVYYCSRWGGDGFYAMDYWGQ
GTLVTVSS
SEQ ID NO: 29: Ab0001 Light chain full length:
DIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIKRTVAAPSVFIFP
PSDEQLKSGTASVVCLLNNFYPREAKVQWKVDNALQSGNSQESVTEQDSKDSTYSLS
STLTLSKADYEKHKVYACEVTHQGLSSPVTKSFNRGECGALPETGG
SEQ ID NO: 30: Ab0001 Heavy chain full length:
EVQLVESGGGLVQPGGSLRLSCAASGFNIKDTYIHWVRQAPGKGLEWVARIYPTNGY
TRYADSVKGRFTISADTSKNTAYLQMNSLRAEDTAVYYCSRWGGDGFYAMDYWGQ
GTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

1. A compound having the structure of formula (II) 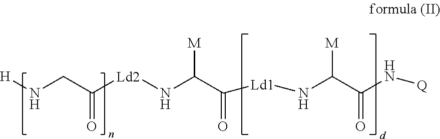
wherein,
Q is hydrogen or LKb-P;
M is hydrogen or LKa-LKb-P;
provided that Q and M are not simultaneously hydrogen;
each LKa is independently selected from 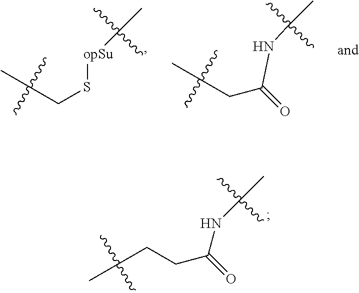
opSu is 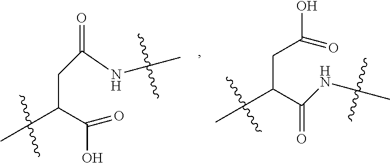 [SMILES: *NC(=O)CC(*)C(=O)O ; *NC(=O)C(*)CC(=O)O]
or a mixture thereof;
each LKb is independently L²-L¹;
P is a payload which is linked to the L¹ moiety;
L¹ is Cleavable sequence 1 comprising an amino acid sequence which can be cleaved by enzyme, and Cleavable sequence 1 comprises 1-10 amino acids;
L² is a bond; or a C₂₋₂₀ alkylene wherein one or more —CH₂— structures in the alkylene is optionally replaced by —CR¹R²—, —O—, —(CO)—, —S(═O)₂—, —NR³—, —N^{⊕}R⁴R⁵—, C₄₋₁₀ cycloalkylene, C₄₋₁₀ heterocyclylene, phenylene; wherein the cycloalkylene, heterocyclylene and phenylene are each independently unsubstituted or substituted with at least one substituent selected from halogen, —C₁₋₁₀ alkyl, —C₁₋₁₀ haloalkyl, —C₁₋₁₀ alkylene-NH—R⁶ and —C₁₋₁₀ alkylene-O—R⁷;
Ld2 and each Ld1 are independently a bond; or selected from —NH—C₁₋₂₀ alkylene-(CO)—, —NH—(PEG); —(CO)—, or is a natural amino acid or oligomeric natural amino acids having a degree of polymerization of 2-10 independently unsubstituted or substituted with -(PEG)ⱼ-R⁸ on the side chain;
-(PEG)ᵢ- and -(PEG)ⱼ- are each a PEG fragment, which comprises the denoted number of consecutive —(O—C₂H₄)— structure units or consecutive —(C₂H₄—O)— structure units, with an optional additional C₁₋₁₀ alkylene at one terminal;
R¹, R², R³, R⁴, R⁵, R⁶, R⁷ are each independently selected from hydrogen, halogen, —C₁₋₁₀ alkyl, —C₁₋₁₀ haloalkyl, C₄₋₁₀ cycloalkylene;
R⁸ is C₁₋₁₀ alkyl;
n is any integer of 2 to 20;
d is 0, or is any integer of 1 to 6;
each i is independently an integer of 1-100, preferably 1 to 20; preferably each i is independently an integer of 1 to 12; more preferably 2 to 8; particularly 4;
each j is independently an integer of 1-100, preferably 1 to 20; preferably each j is independently an integer of 1 to 12; more preferably 8 to 12; particularly 8 or 12.

2. The compound of claim 1, wherein P has the structure of formula (i) 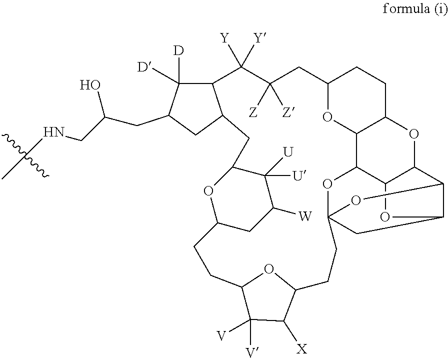
wherein,
each of D and D′ is independently selected from R⁹ and OR⁹, wherein R⁹ is H, C₁₋₃ alkyl, or C₁₋₃ haloalkyl;
each of U and U′ is independently H, C₁₋₃ alkoxy, or C₁₋₃ alkyl; or U and U′ taken together are ═CH₂;
W is C₁₋₃ alkyl;
each of V and V′ is independently H, C₁₋₃ alkoxy, or C₁₋₃ alkyl; or U and U′ taken together are ═CH₂;
X is H or C₁₋₆ alkoxy;
each of Y and Y′ is independently H or C₁₋₃ alkoxy; or Y and Y′ taken together are ═O, ═CH₂; and
each of Z and Z′ is independently H or C₁₋₃ alkoxy; or Z and Z′ taken together are ═O, ═CH₂;
or a pharmaceutically acceptable salt thereof;
preferably, P has the structure of formula (i-1) 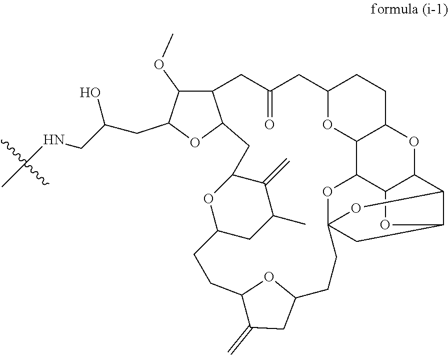 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]
further preferably, P has the structure of formula (ii) 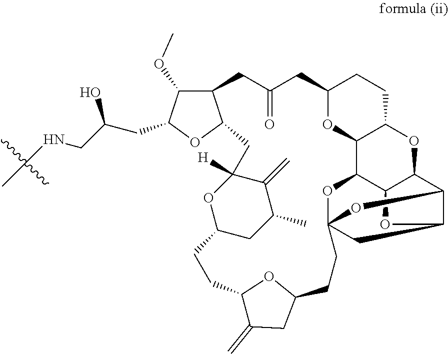 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]
wherein the wavy bond shows the connection site for connection to the L¹ moiety.

3. The compound of claim 1, having the structure of formula (II-i)

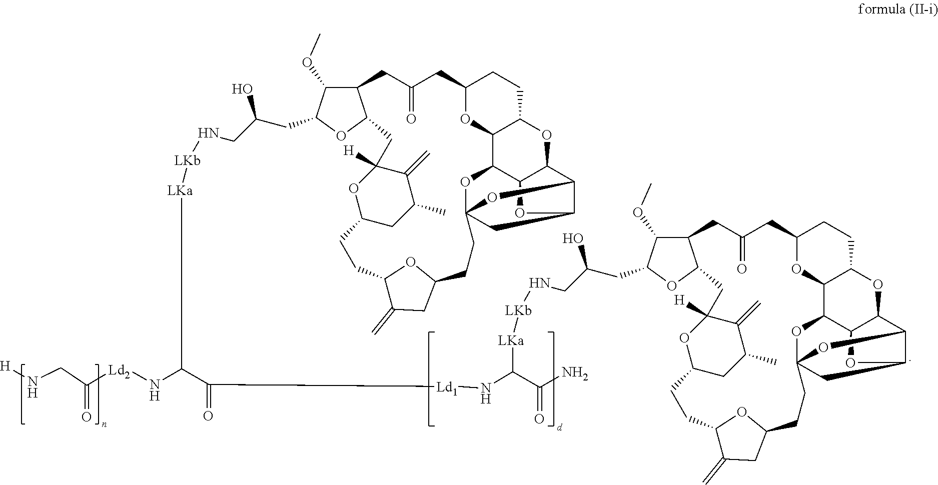

4. The compound of claim 1, selected from

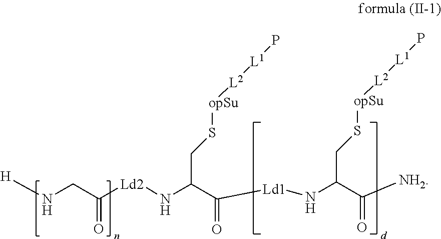

5. The compound of claim 1, selected from 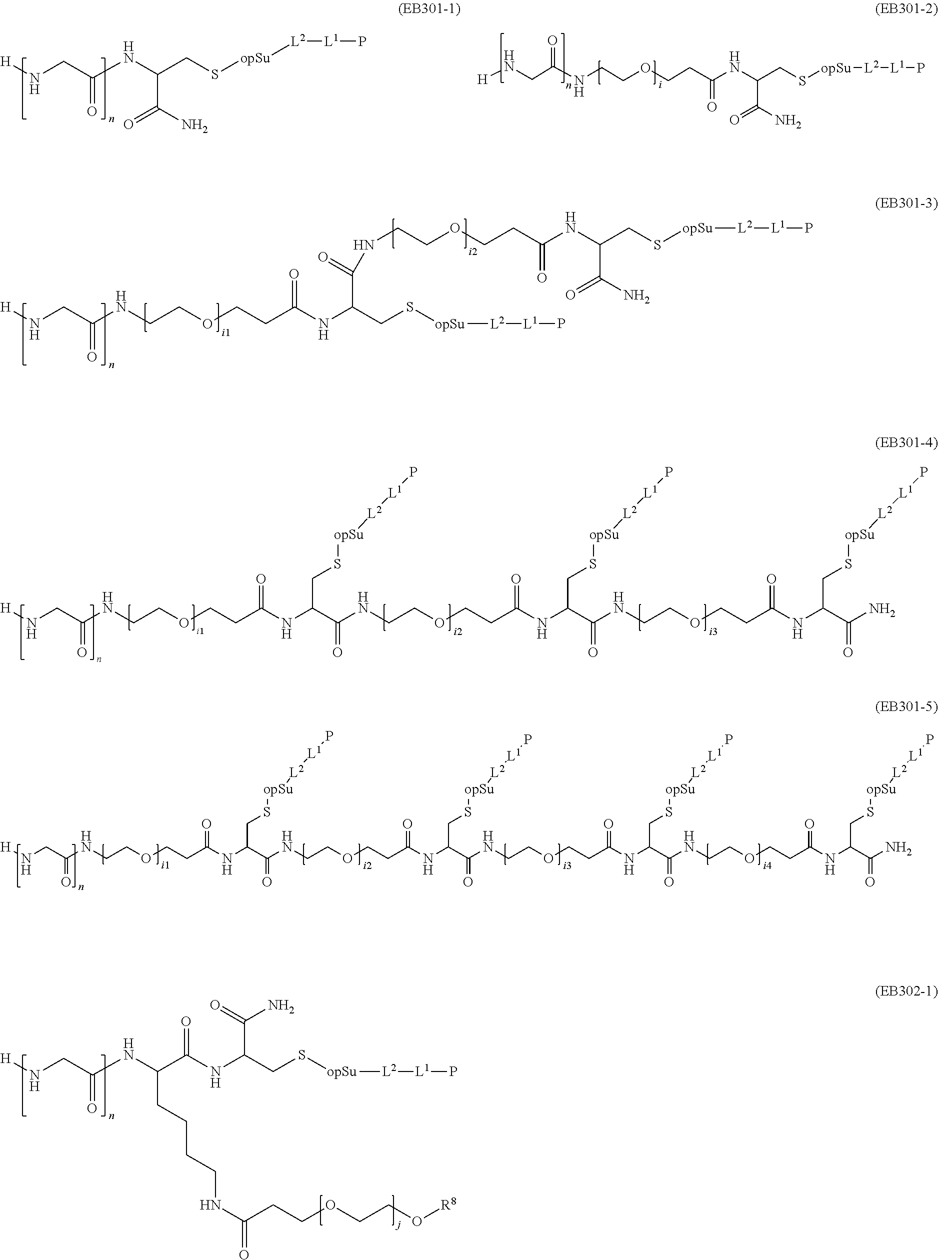 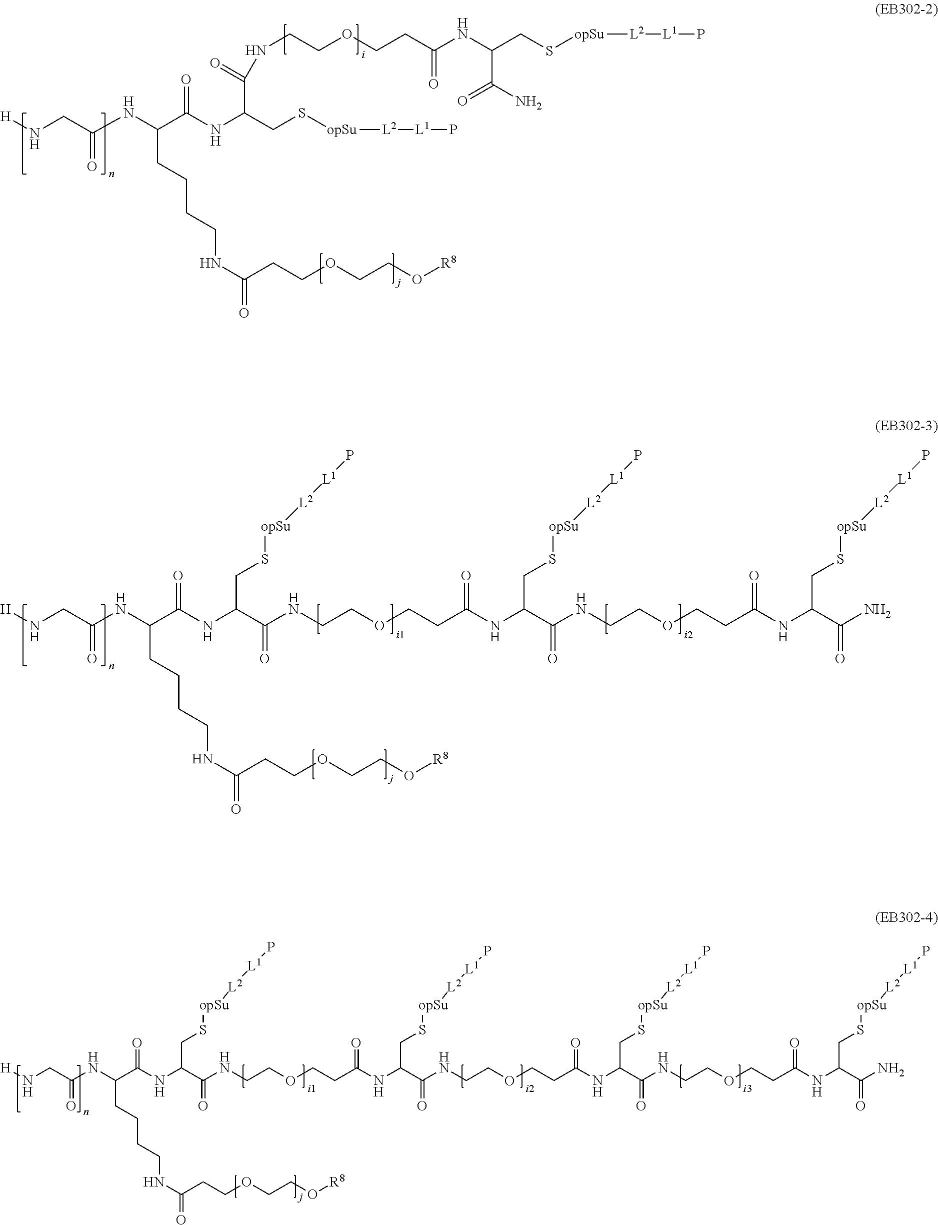
wherein, each i, i1, i2, i3, i4 is independently an integer of 1-100, preferably 1 to 20; preferably each i, i1, i2, i3, i4 is independently an integer of 1 to 12; more preferably 2 to 8; particularly 4.

6. The compound of claim 1, wherein Ld2 and each Ld1 are independently a bond or 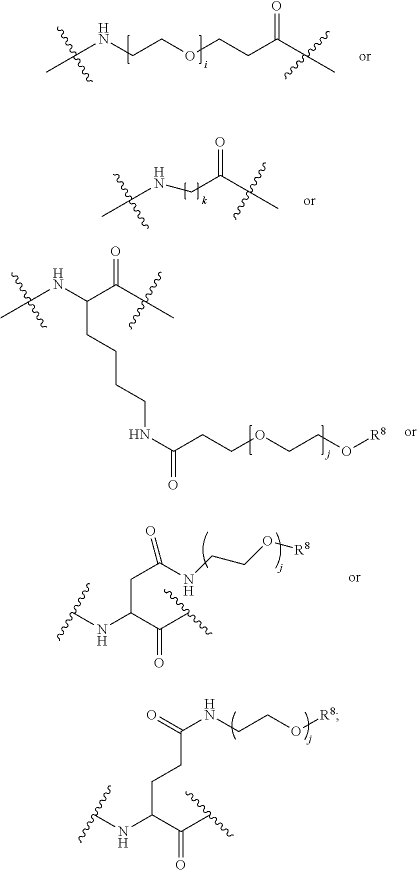
each k is independently an integer of 1-100, preferably 1 to 20; preferably each k is independently an integer of 1 to 12; more preferably 1 to 7; particularly 1, or 3 or 5.

7. The compound of claim 1, wherein
Cleavable sequence 1 is selected from GLy-GLy-Phe-GLy, Phe-Lys, Val-Cit, Val-Lys, GLy-Phe-Leu-Gly, Ala-Leu-Ala-Leu, Ala-Ala-Ala, Val-Cit-PABC and the combination thereof; preferably, Cleavable sequence 1 is GLy-GLy-Phe-Gly or Val-Cit-PABC.

8. The compound of claim 1, wherein
Q is hydrogen; and/or
R⁸ is C₁₋₆ alkyl, preferably methyl; and/or
n is an integer of 2 to 5, especially 3; and/or
d is 0, or is any integer of 1 to 4; preferably 0, 1, 2 or 3.

9. The compound of claim 1, selected 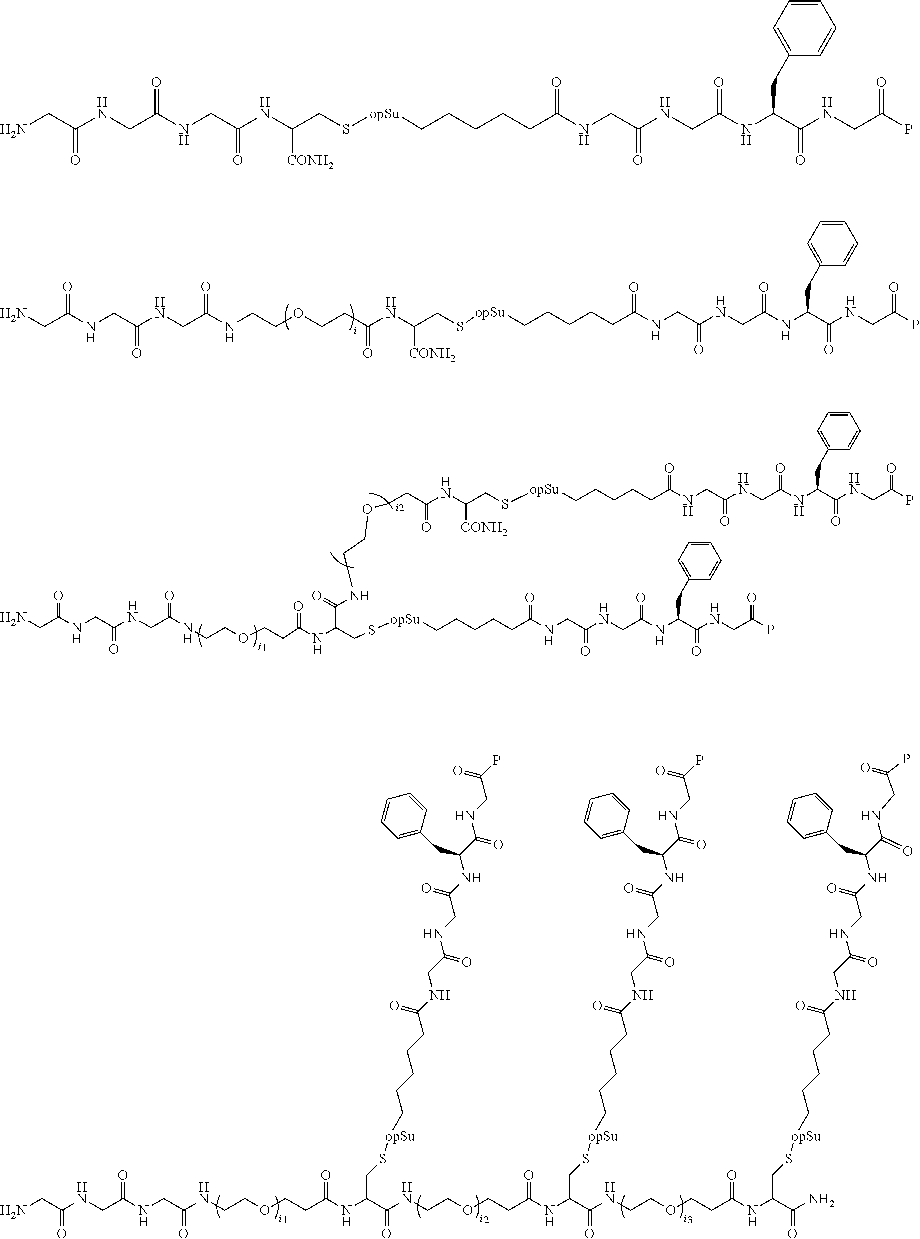 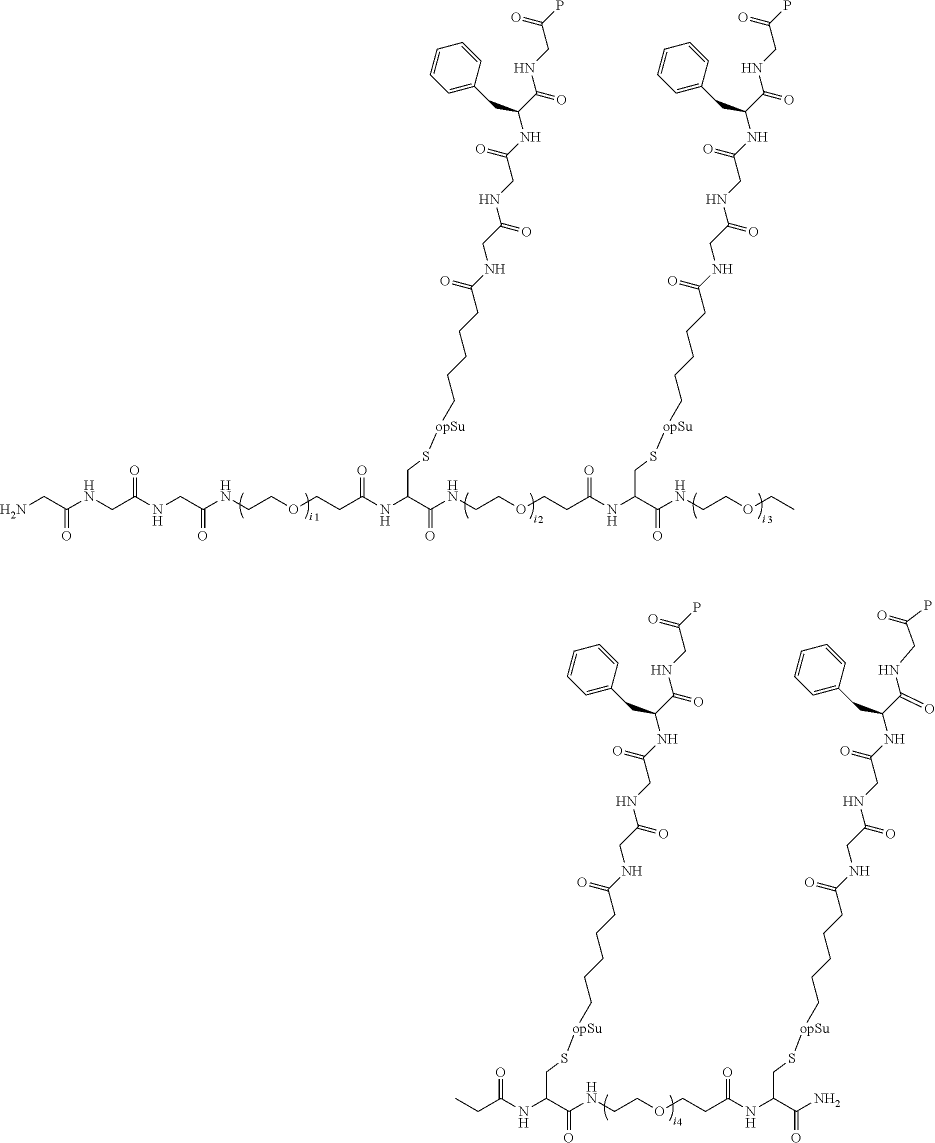 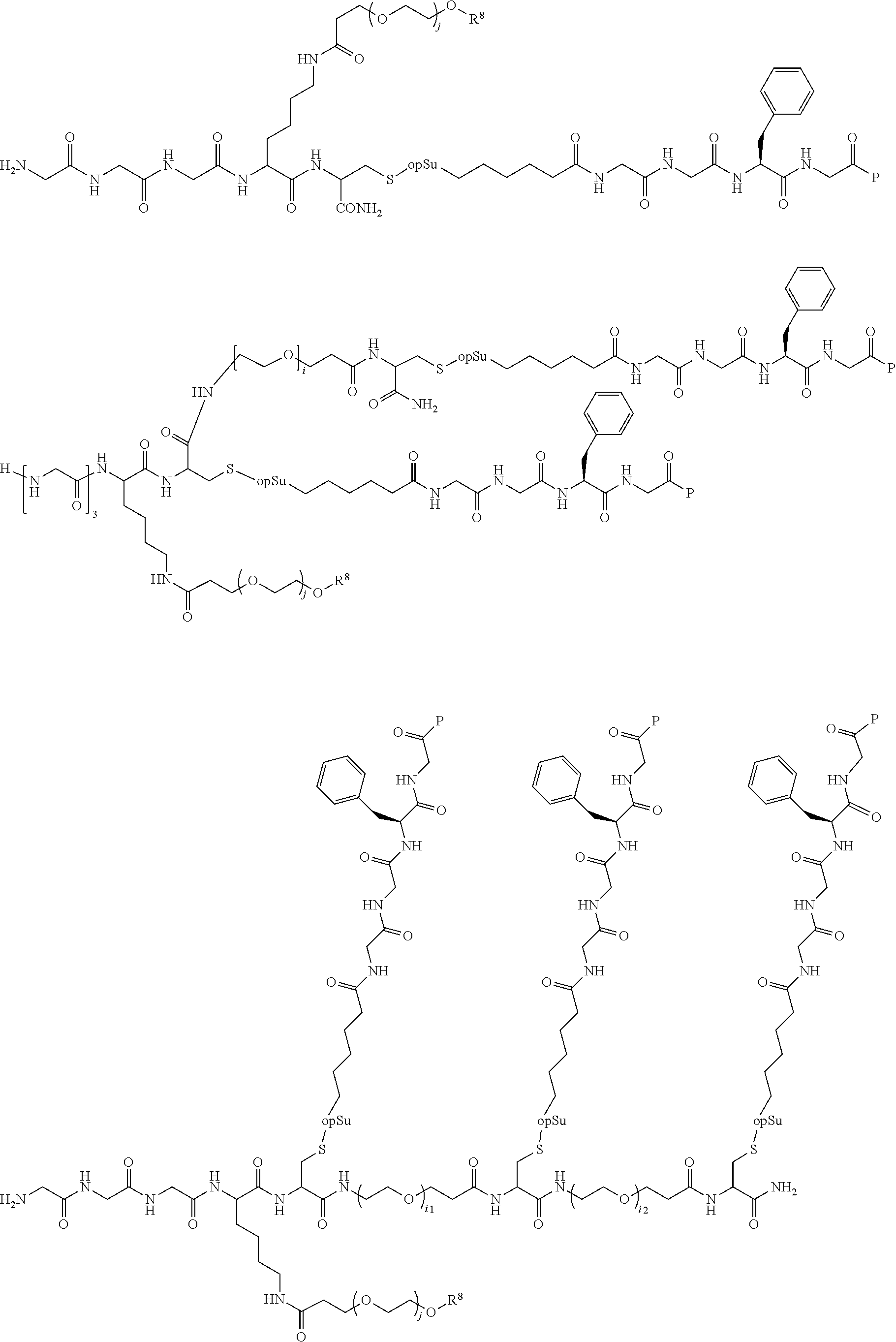 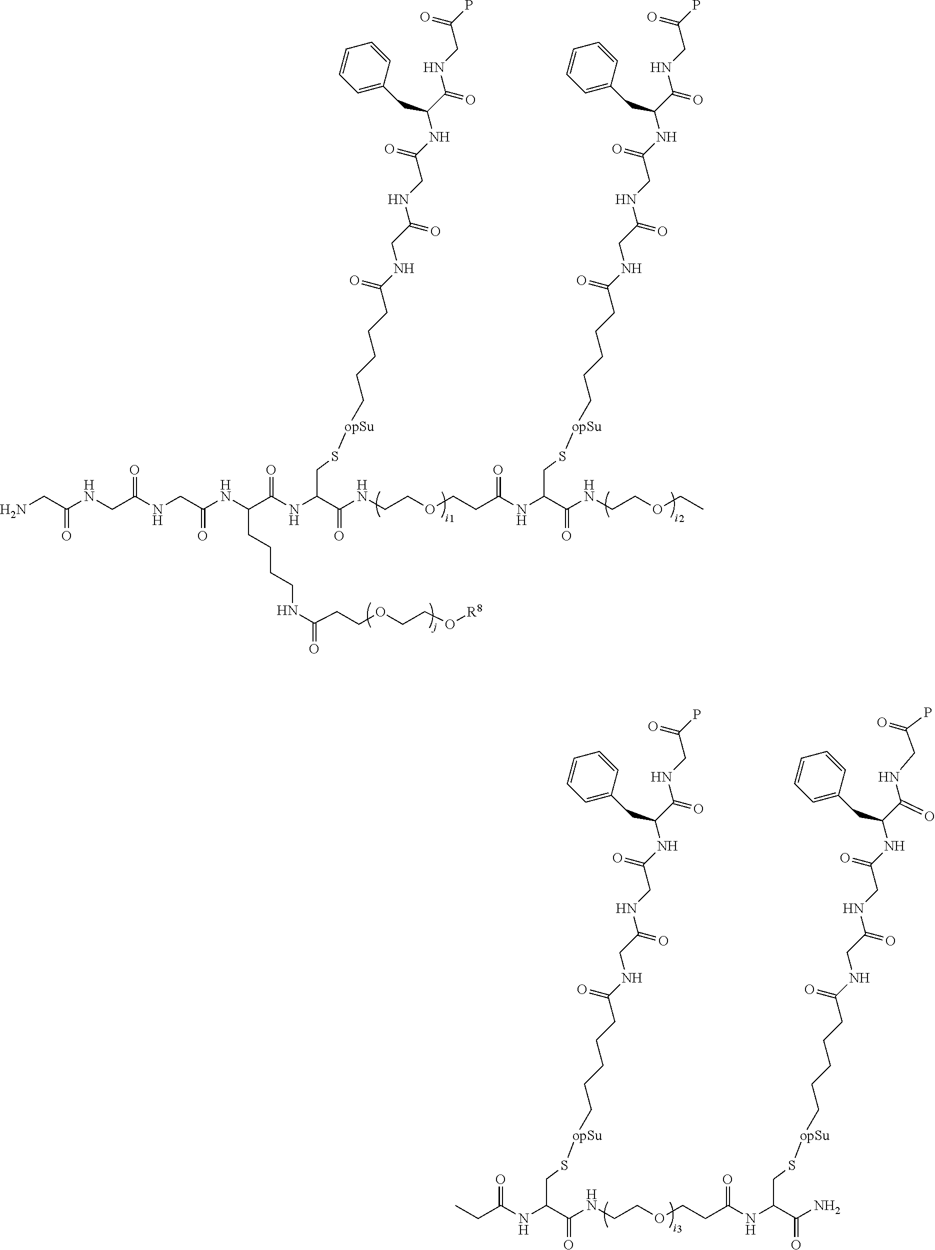 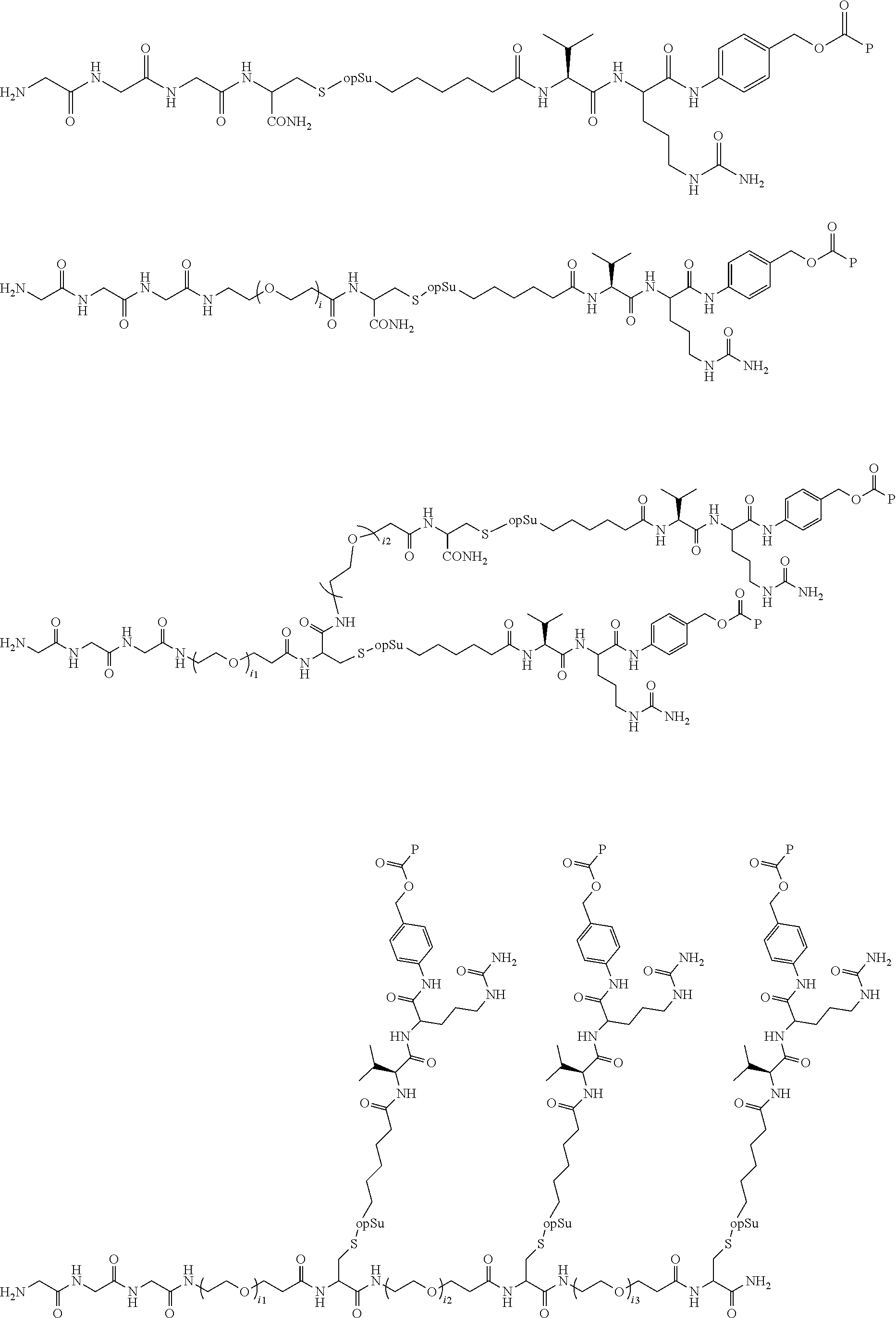 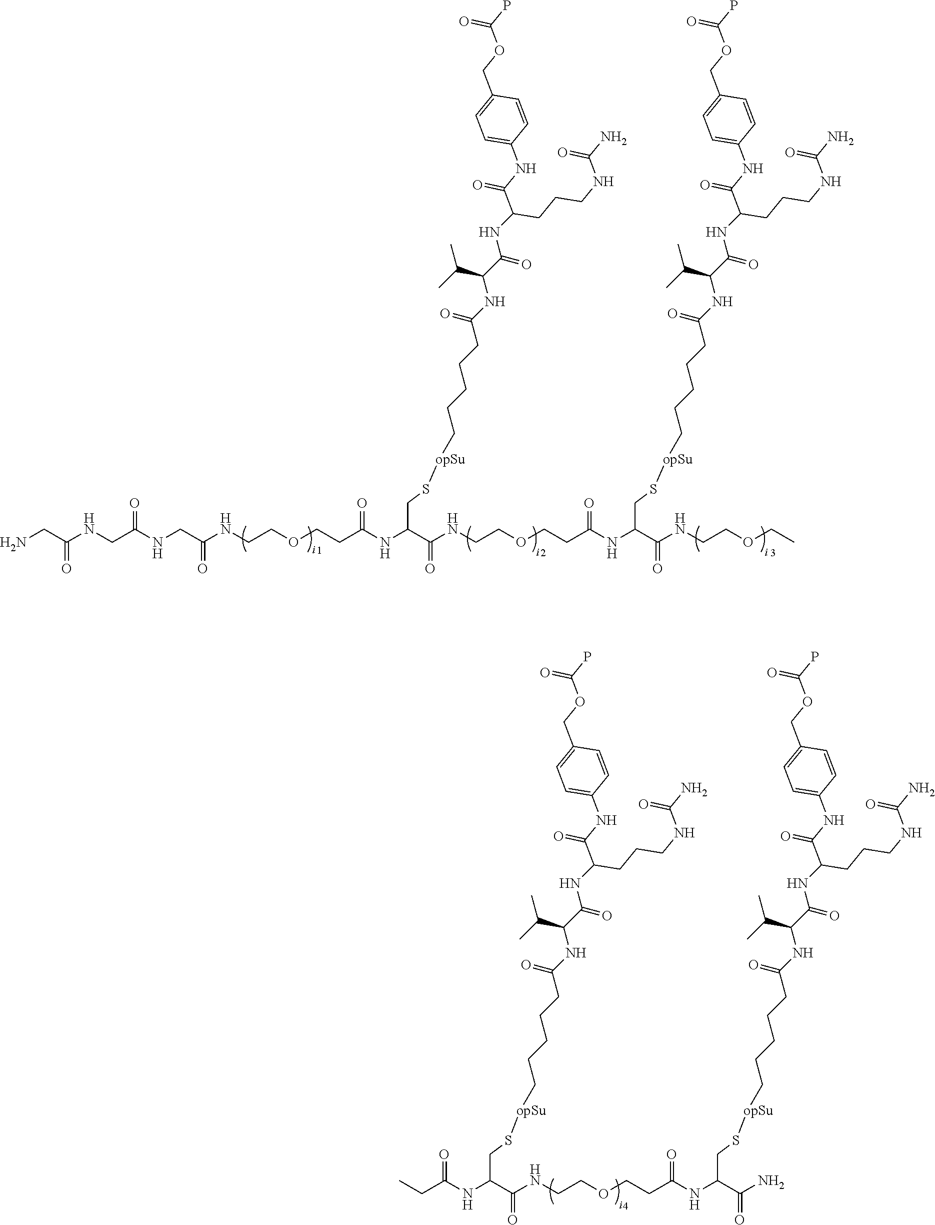 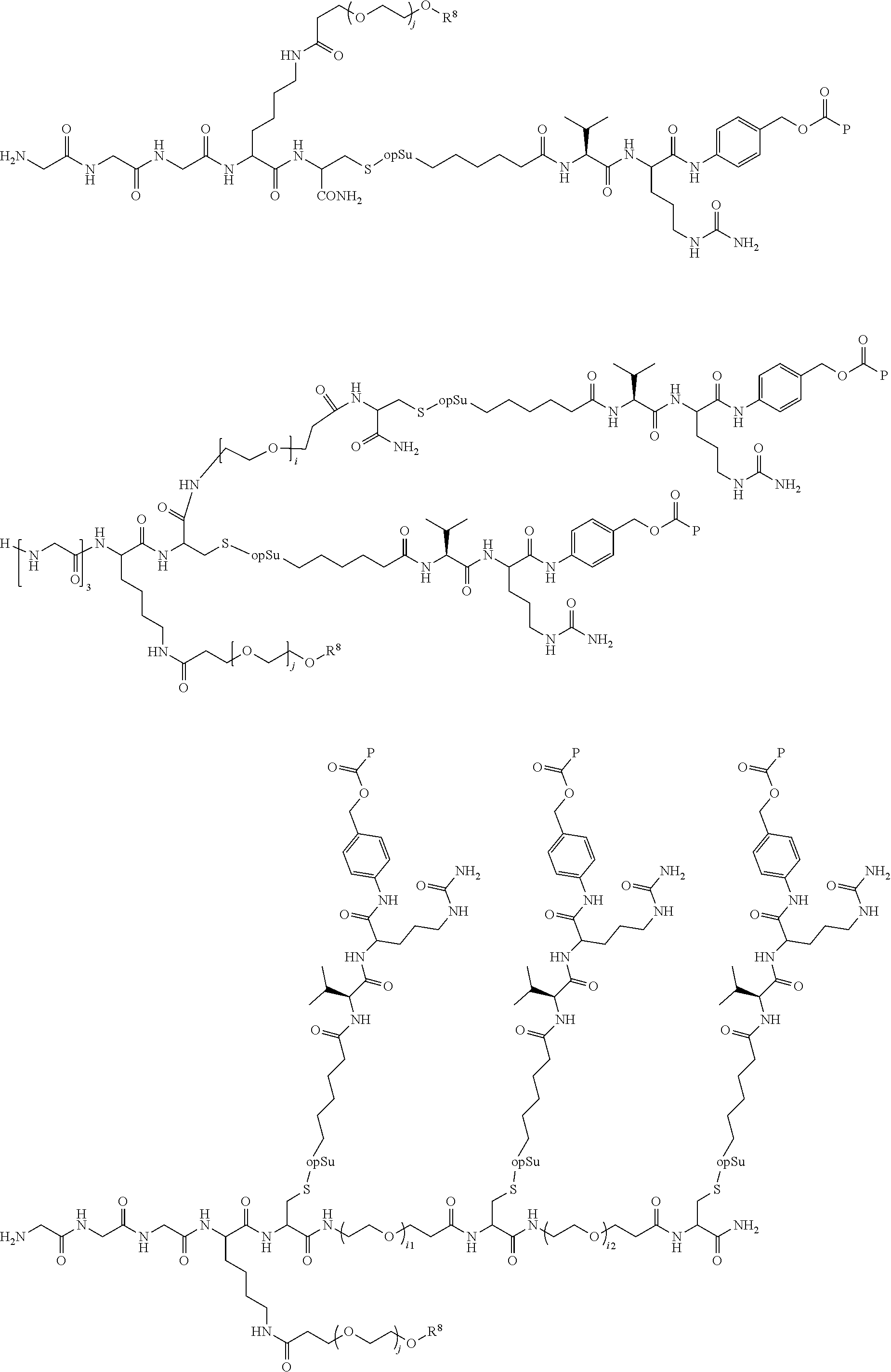 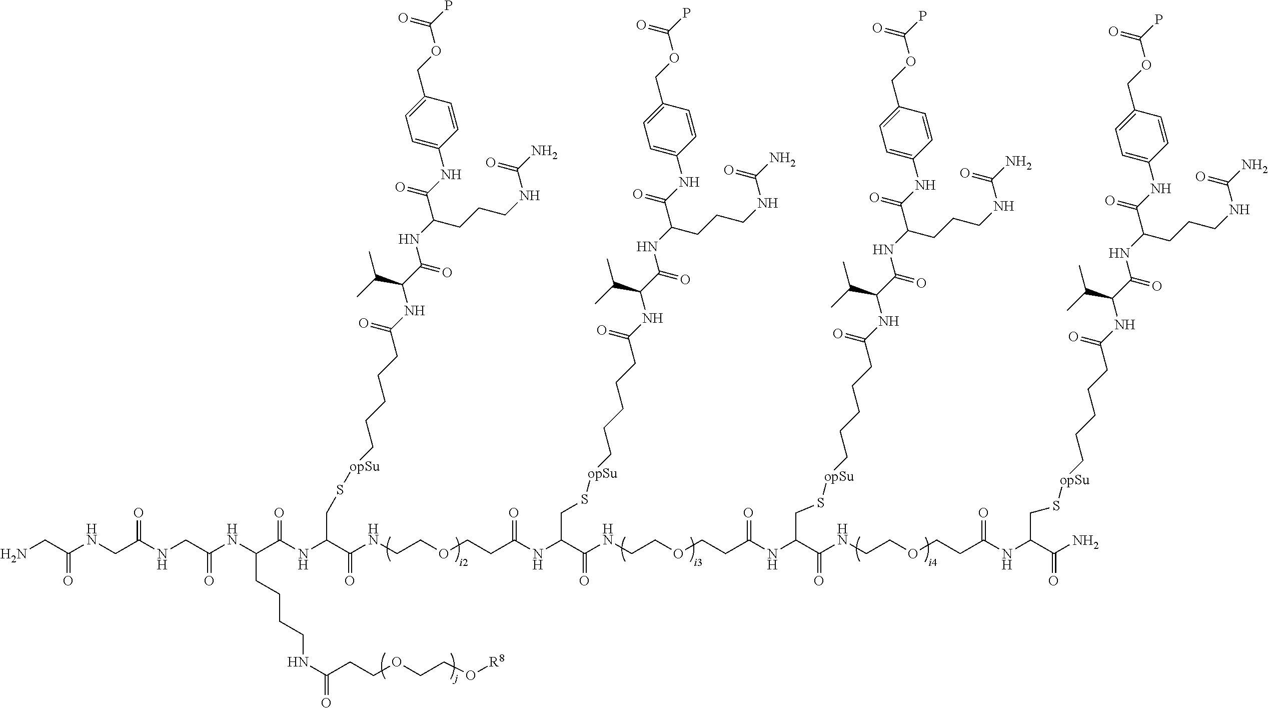
each i, i1, i2, i3, i4 is independently an integer of 1-100, preferably 1 to 20; preferably each i, i1, i2, i3, i4 is independently an integer of 1 to 12; more preferably 2 to 8; particularly 4.

10. A conjugate having the structure of formula (III): 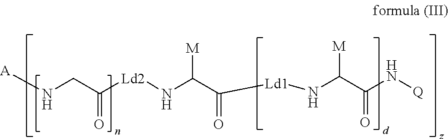
wherein,
Q is hydrogen or LKb-P;
M is hydrogen or LKa-LKb-P;
provided that Q and M are not simultaneously hydrogen;
A is a targeting molecule which is linked to the Gₙ moiety of the compound of formula (II); G is glycine; 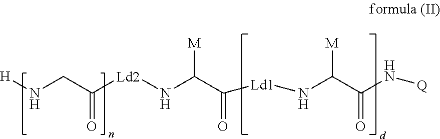
z is an integer of 1 to 20;
each LKa is independently selected from 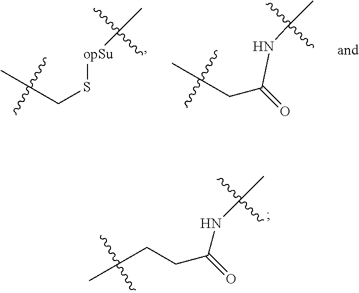
opSu is 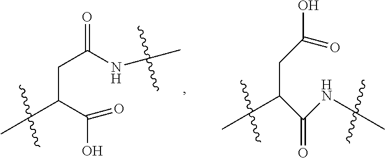 [SMILES: *NC(=O)CC(*)C(=O)O ; *NC(=O)C(*)CC(=O)O]
or a mixture thereof;
each LKb is independently L²-L¹;
P is a payload which is linked to the L¹ moiety;
L¹ is Cleavable sequence 1 comprising an amino acid sequence which can be cleaved by enzyme, and Cleavable sequence 1 comprises 1-10 amino acids;
L² is a bond; or a C₂₋₂₀ alkylene wherein one or more —CH₂— structures in the alkylene is optionally replaced by —CR¹R²—, —O—, —(CO)—, —S(═O)₂—, —NR³, —N^{⊕}R⁴R⁵, C₄₋₁₀ cycloalkylene, C₄₋₁₀ heterocyclylene, phenylene; wherein the cycloalkylene, heterocyclylene and phenylene are each independently unsubstituted or substituted with at least one substituent selected from halogen, —C₁₋₁₀ alkyl, —C₁₋₁₀ haloalkyl, —C₁₋₁₀ alkylene-NH—R⁶ and —C₁₋₁₀ alkylene-O—R⁷;
Ld2 and each Ld1 are independently a bond; or selected from —NH—C₁₋₂₀ alkylene-(CO)—, —NH—(PEG); —(CO)—, or is a natural amino acid or oligomeric natural amino acids having a degree of polymerization of 2-10 independently unsubstituted or substituted with -(PEG)ⱼ-R⁸ on the side chain;
-(PEG)ᵢ- and —(PEG)ⱼ- are each a PEG fragment, which comprises the denoted number of consecutive —(O—C₂H₄)— structure units or consecutive —(C₂H₄—O)— structure units, with an optional additional C₁₋₁₀ alkylene at one terminal;
R¹, R², R³, R⁴, R⁵, R⁶, R⁷ are each independently selected from hydrogen, halogen, —C₁₋₁₀ alkyl, —C₁₋₁₀ haloalkyl, C₄₋₁₀ cycloalkylene;
R⁸ is C₁₋₁₀ alkyl;
n is any integer of 2 to 20;
d is 0, or is any integer of 1 to 6;
each i is independently an integer of 1-100, preferably 1 to 20; preferably each i is independently an integer of 1 to 12; more preferably 2 to 8; particularly 4;
each j is independently an integer of 1-100, preferably 1 to 20; preferably each j is independently an integer of 1 to 12; more preferably 8 to 12; particularly 8 or 12.

11. The conjugate of claim 10, wherein the payload is a cytotoxin or a fragment thereof, with an optional derivatization in order to connect to the L¹ moiety;
preferably, the payload is selected from the group consisting of the following compounds or a fragment thereof:
taxanes, maytansinoids, auristatins, epothilones, combretastatin A-4 phosphate, combretastatin A-4 and derivatives thereof, indol-sulfonamides, vinblastines such as vinblastine, vincristine, vindesine, vinorelbine, vinflunine, vinglycinate, anhy-drovinblastine, dolastatin 10 and analogues, halichondrin B, cribulin, indole-3-oxoacetamide, podophyllotoxins, 7-diethylamino-3-(2′-benzoxazolyl)-coumarin (DBC), discodermolide, laulimalide, camptothecins and derivatives thereof, mitoxantrone, mitoguazone, nitrogen mustards, nitrosoureasm, aziridines, benzodopa, carboquone, meturedepa, uredepa, dynemicin, esperamicin, neocarzinostatin, aclacinomycin, actinomycin, antramycin, bleomycins, actinomycin C, carabicin, carminomycin, cardinophyllin, carminomycin, actinomycin D, daunorubicin, detorubicin, adriamycin, epirubicin, esorubicin, idarubicin, marcellomycin, mitomycins, nogalamycin, olivomycin, peplomycin, porfiromycin, puromycin, ferric adriamycin, rodorubicin, rufocromomycin, streptozocin, zinostatin, zorubicin, trichothecene, T-2 toxin, verracurin A, bacillocporin A, anguidine, ubenimex, azaserine, 6-diazo-5-oxo-L-norleucine, dimethyl folic acid, methotrexate, pteropterin, trimetrexate, edatrexate, fludarabine, 6-mercaptopurine, tiamiprine, thioguanine, ancitabine, gemcitabine, enocitabine, azacitidine, 6-azauridine, carmofur, cytarabine, dideoxyuridine, doxifluridine, floxuridine, calusterone, dromostanolone propionate, epitiostanol, mepitiostane, testolactone, aminoglutethimide, mitotane, trilostane, flutamide, nilutamide, bicalutamide, leuprorelin acetate, protein kinase inhibitors and a proteasome inhibitors; preferably eribulin; and/or
selected from vinblastines, colchicines, taxanes, auristatins, maytansinoids, calicheamicin, doxonubicin, duocarmucin, SN-38, cryptophycin analogue, deruxtecan, duocarmazine, calicheamicin, centanamycin, dolastansine, pyrrolobenzodiazepine, exatecan and derivatives thereof; and/or
selected from auristatins, especially MMAE, MMAF or MMAD; and/or
selected from exatecan and derivatives thereof, such as DX8951f; and/or
selected from DXd-(1) and DXd-(2); preferably DXd-(1).

12. The conjugate of claim 10, wherein P has the structure of formula (i) 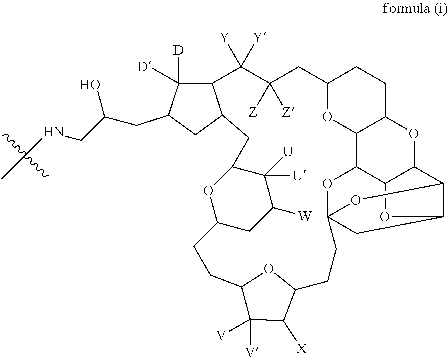
wherein,
each of D and D′ is independently selected from R⁹ and OR⁹, wherein R⁹ is H, C₁₋₃ alkyl, or C₁₋₃ haloalkyl;
each of U and U′ is independently H, C₁₋₃ alkoxy, or C₁₋₃ alkyl; or U and U′ taken together are ═CH₂;
W is C₁₋₃ alkyl;
each of V and V′ is independently H, C₁₋₃ alkoxy, or C₁₋₃ alkyl; or U and U′ taken together are ═CH₂;
X is H or C₁₋₆ alkoxy;
each of Y and Y′ is independently H or C₁₋₃ alkoxy; or Y and Y′ taken together are ═O, ═CH₂; and
each of Z and Z′ is independently H or C₁₋₃ alkoxy; or Z and Z′ taken together are ═O, ═CH₂;
or a pharmaceutically acceptable salt thereof;
preferably, P has the structure of formula (i-1) 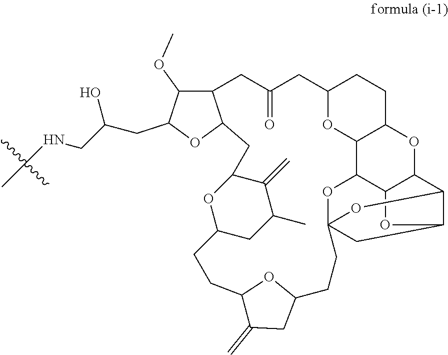 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]
further preferably, P has the structure of formula (ii) 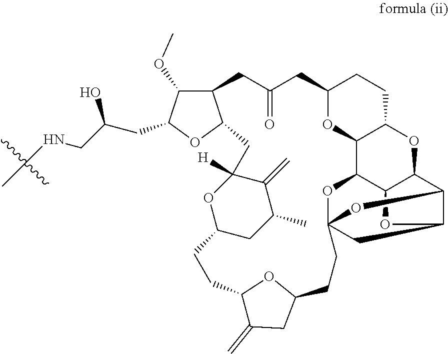 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]
wherein the wavy bond shows the connection site for connection to the L¹ moiety;
and/or
(ii) the targeting molecule is antibody or antigen binding fragment thereof; preferably, antibody is anti-CD19 antibody, anti-CD20 antibody, anti-CD22 antibody, anti-CD25 antibody, anti-CD30/TNFRSF8 antibody, anti-CD33 antibody, anti-CD37 antibody, anti-CD44v6 antibody, anti-CD56 antibody, anti-CD70 antibody, anti-CD71 antibody, anti-CD74 antibody, anti-CD79b antibody, anti-CD117/KITk antibody, anti-CD123 antibody, anti-CD138 antibody, anti-CD142 antibody, anti-CD174 antibody, anti-CD227/MUC1 antibody, anti-CD352 antibody, anti-CLDN18.2 antibody, anti-DLL3 antibody, anti-ErbB2/HER2 antibody, anti-CN33 antibody, anti-GPNMB antibody, anti-ENPP3 antibody, anti-Nectin-4 antibody, anti-EGFRvIII antibody, anti-SLC44A4/AGS-5 antibody, anti-CEACAM5 antibody, anti-PSMA antibody, anti-TIM1 antibody, anti-LY6E antibody, anti-LIV1 antibody, anti-Nectin4 antibody, anti-SLITRK6 antibody, anti-HGFR/cMet antibody, anti-SLAMF7/CS1 antibody, anti-EGFR antibody, anti-BCMA antibody, anti-AXL antibody, anti-NaPi2B antibody, anti-GCC antibody, anti-STEAP1 antibody, anti-MUC16 antibody, anti-Mesothelin antibody, anti-ETBR antibody, anti-EphA2 antibody, anti-5T4 antibody, anti-FOLR1 antibody, anti-LAMP1 antibody, anti-Cadherin 6 antibody, anti-FGFR2 antibody, anti-FGFR3 antibody, anti-CA6 antibody, anti-CanAg antibody, anti-integrin αV antibody, anti-TDGF1 antibody, anti-Ephrin A4 antibody, anti-TROP2 antibody, anti-PTK7 antibody, anti-NOTCH3 antibody, anti-C4.4A antibody, anti-FLT3 antibody, anti-B7H3/4 antibody, anti-Tissue Factor antibody, or anti-ROR1/2 antibody; more preferably, anti-HER2 antibody, anti-FGFR3 antibody, anti-Trop2 antibody, anti-HER3 antibody or anti-FRα antibody; more preferably, the antibody is anti-FGFR3 antibody, anti-Trop2 antibody, anti-HER3 antibody or anti-FRα antibody;
further preferably, the antibody is an anti-human FGFR3 antibody, preferably Ab0206; or an anti-human HER2 antibody, preferably Ab0036 or Ab0001.

13. The conjugate of claim 10, having the structure of formula (III-i)

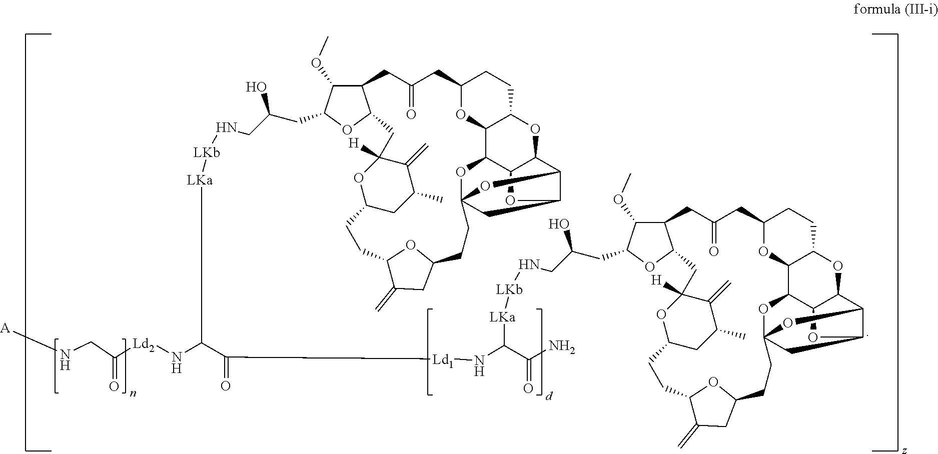

14. The conjugate of claim 10, wherein
the conjugate has the structure of the following formula (III-1):

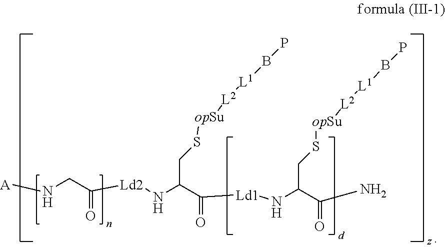

15. The conjugate of claim 10, wherein
the conjugate has the structure of the following: 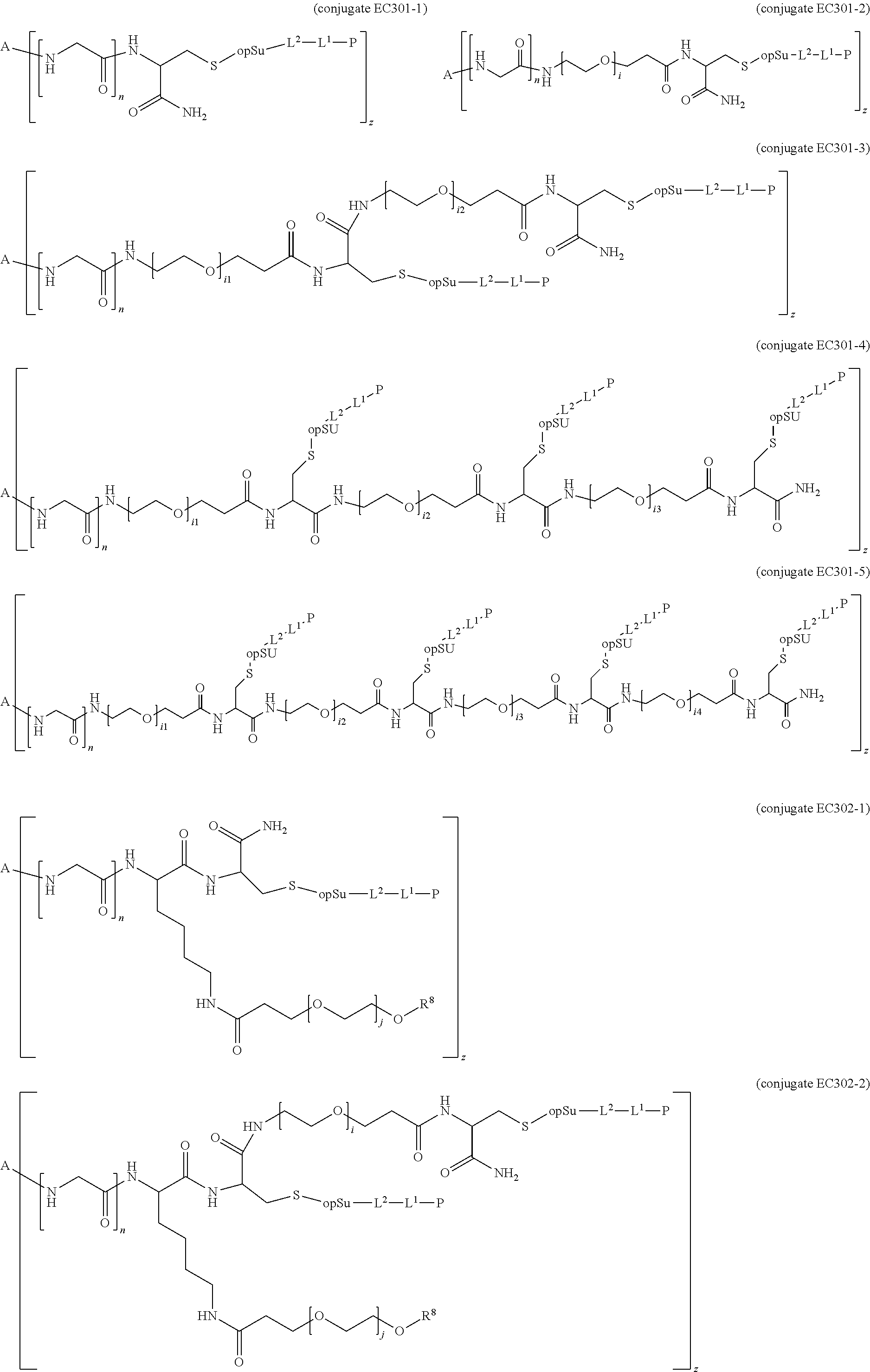 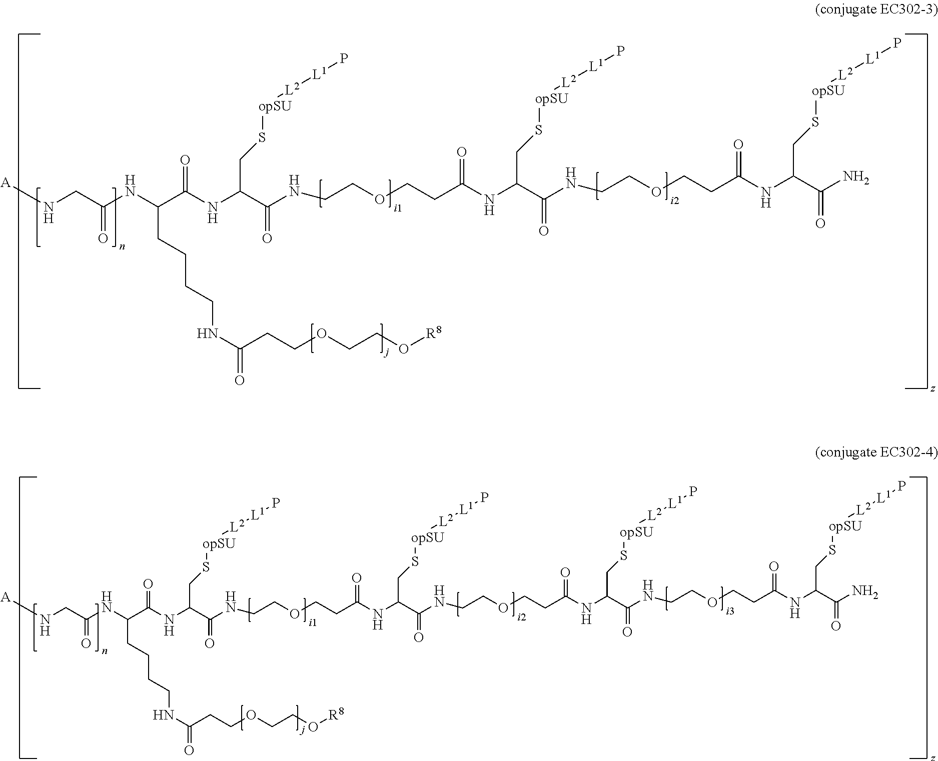
preferably, z is 1 to 4; preferably 2;
each i, i1, i2, i3, i4 is independently an integer of 1-100, preferably 1 to 20; preferably each i, i1, i2, i3, i4 is independently an integer of 1 to 12; more preferably 2 to 8; particularly 4;
each j is independently an integer of 1-100, preferably 1 to 20; preferably each j is independently an integer of 1 to 12; more preferably 8 to 12; particularly 8 or 12;
preferably, n is 3, L² is —(CH₂)ₚ−(CH₂)₂(CO)—, p is 3, L¹ is GGFG or Val-Cit-PABC.

16. The conjugate of claim 10, wherein
the targeting molecule is an antibody or an antigen binding fragment thereof; the antibody or antigen binding fragment is preferably modified to connect with the Gₙ moiety in the compound of formula (II);
preferably,
(i) the antibody is an anti-human HER2 antibody or anti-human FGFR3 antibody; and/or
(ii) the Gₙ moiety in the compound of formula (II) is the recognition sequence of the ligase acceptor substrate of a ligase; and
the antibody is modified to comprises the recognition sequence of the ligase donor substrate in order to connect with the Gₙ moiety in the compound of formula (II);
preferably,
(a) the ligase is a Sortase selected from Sortase A, Sortase B, Sortase C, Sortase D and Sortase L. plantarum; preferably Sortase A from Staphylococcus aureus; and/or
(b) the recognition sequence of the ligase donor substrate is LPXTGJ, J is absent or is Gₘ, wherein G is glycine, m is an integer of 1 to 10; preferably, the recognition sequence of the ligase donor substrate is LPXTG or LPETGG; and/or
the connection of LPXTGJ with Gₙ results in LPXTGₙ.

17. The conjugate of claim 10, wherein

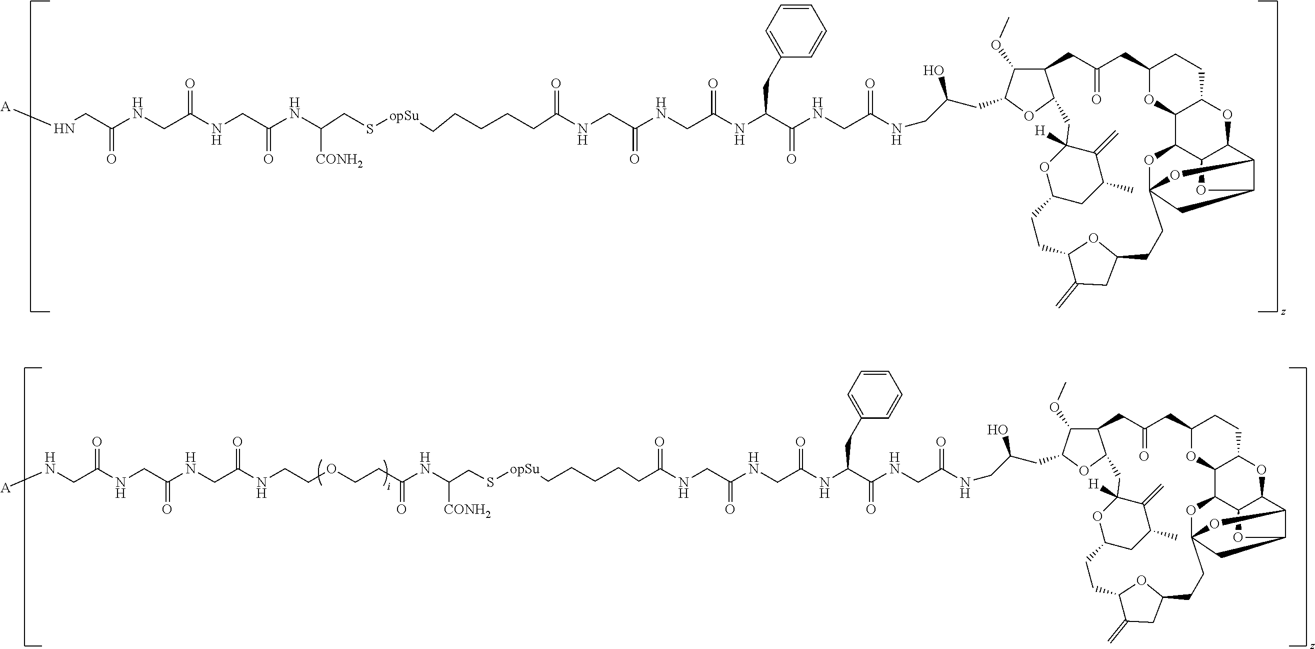

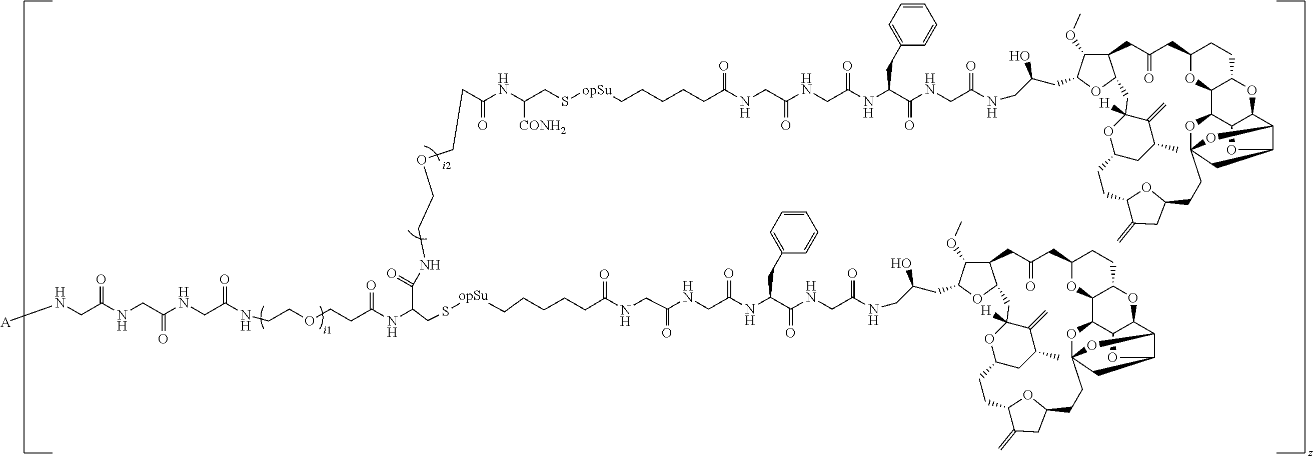

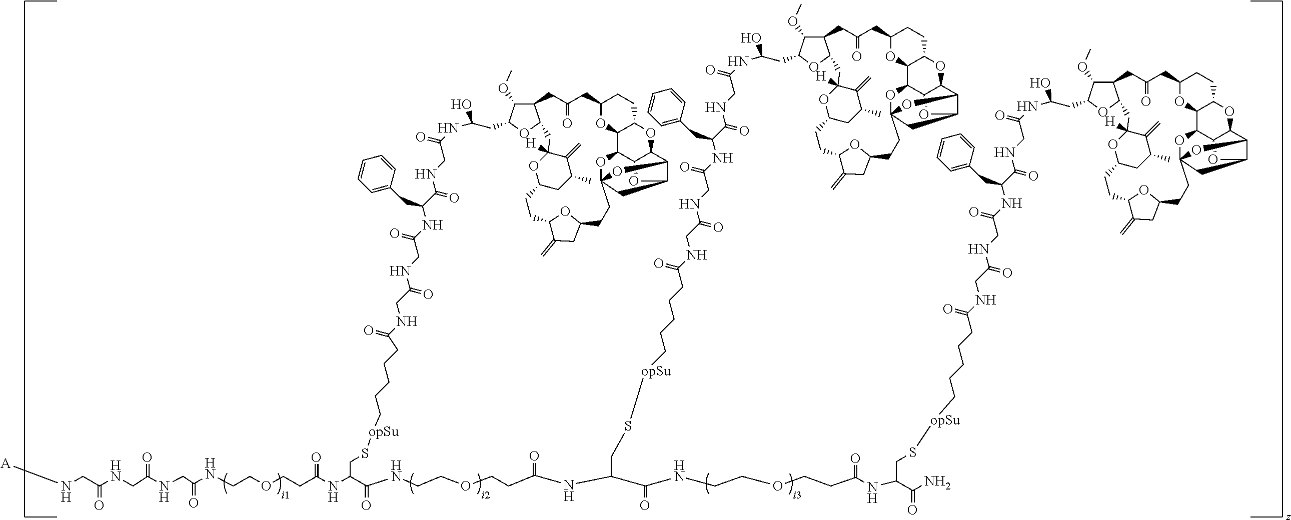

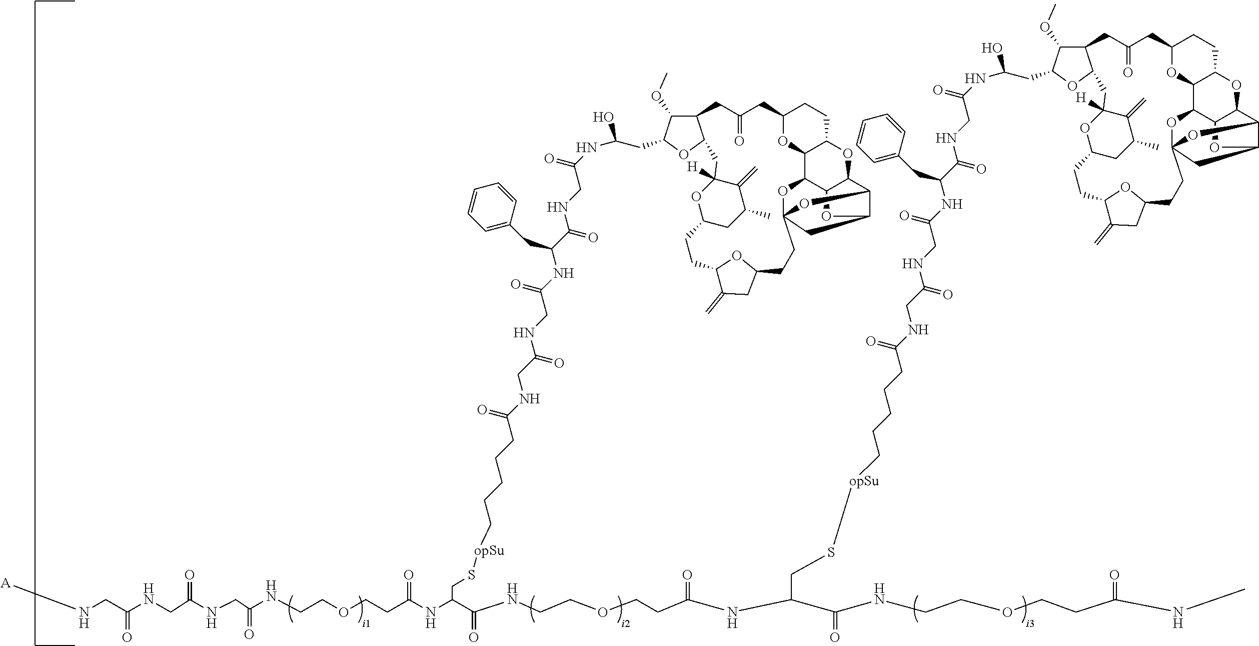

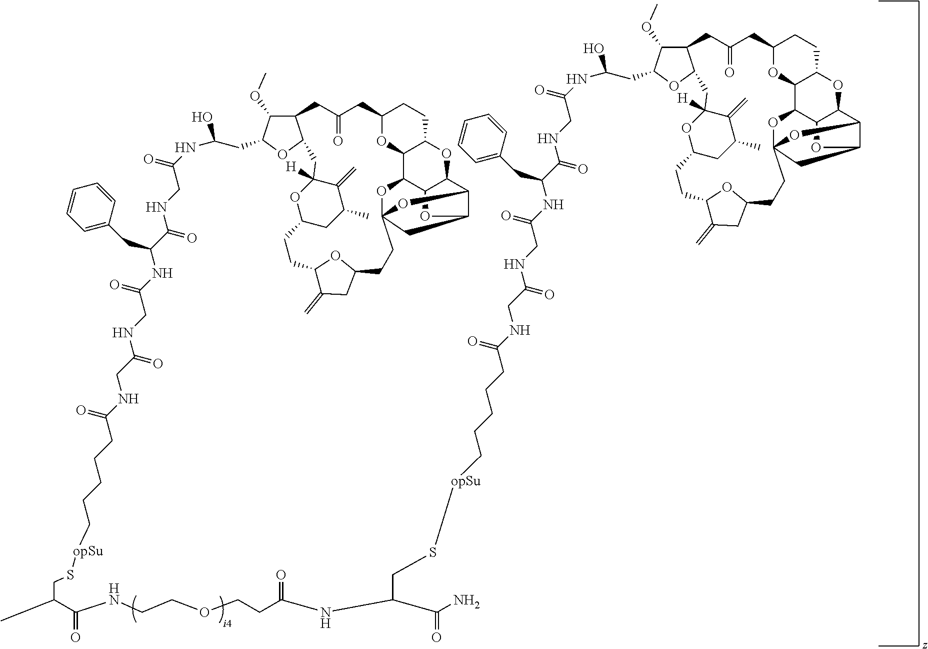

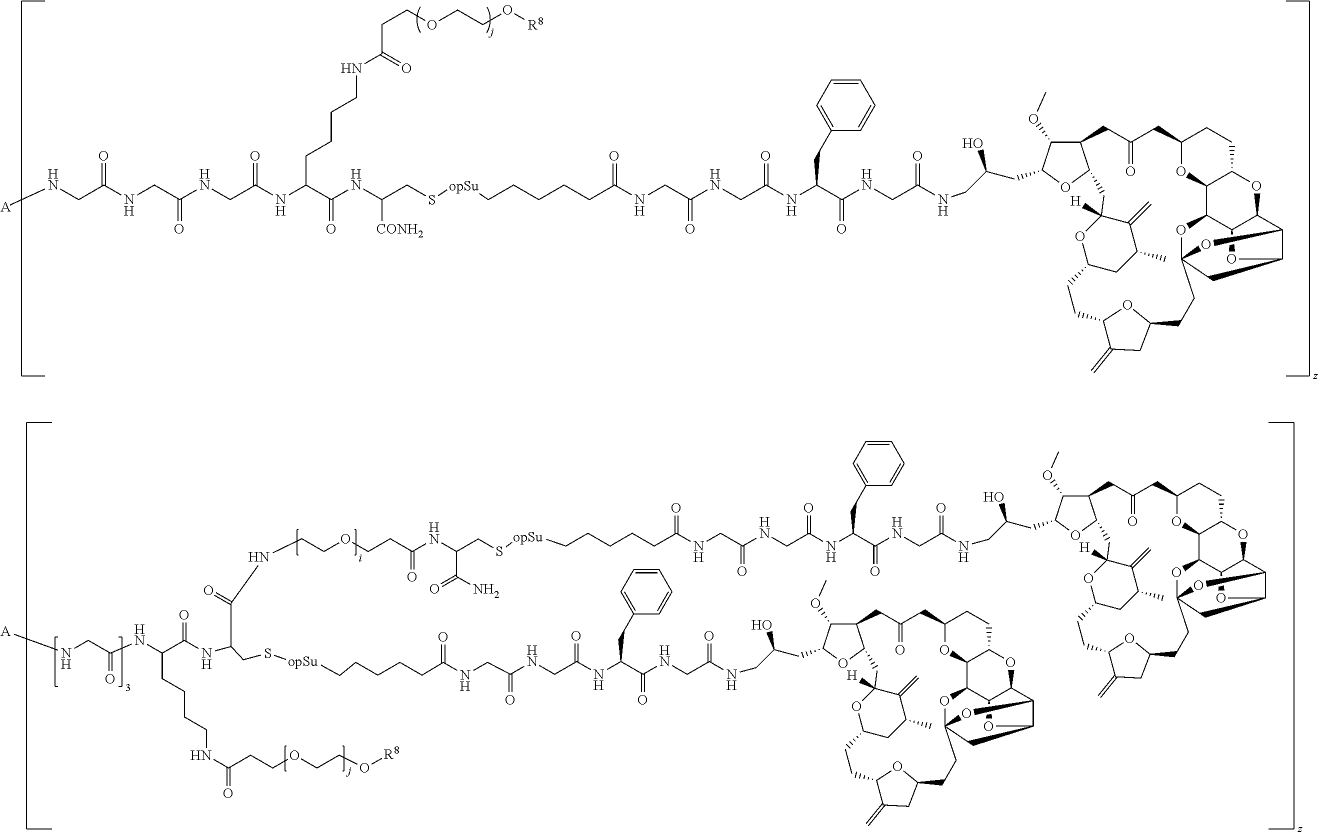

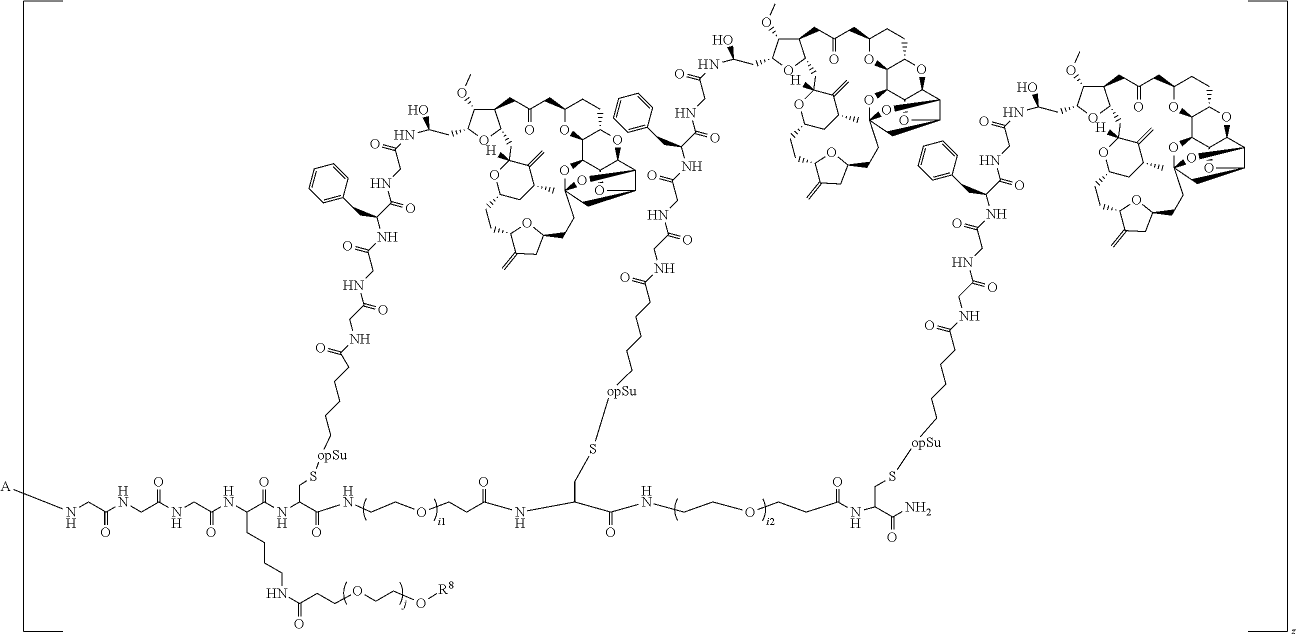

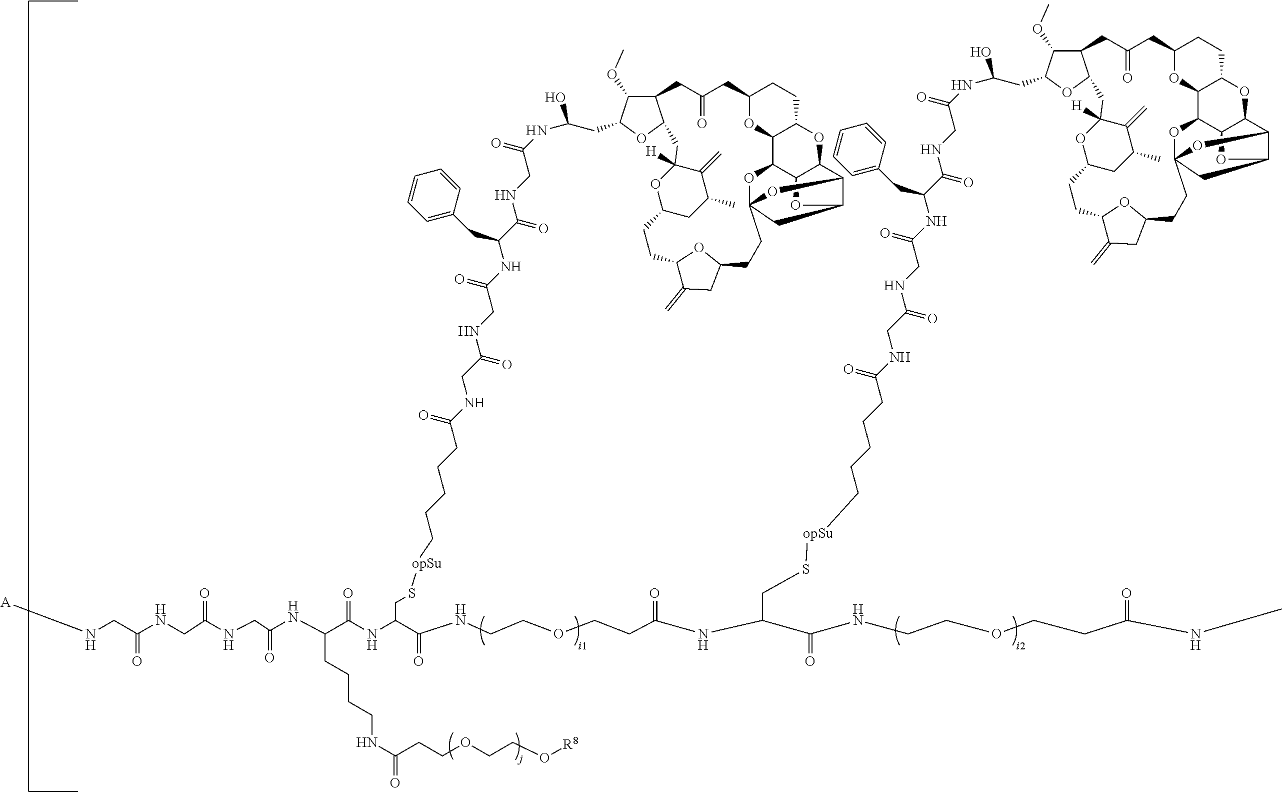

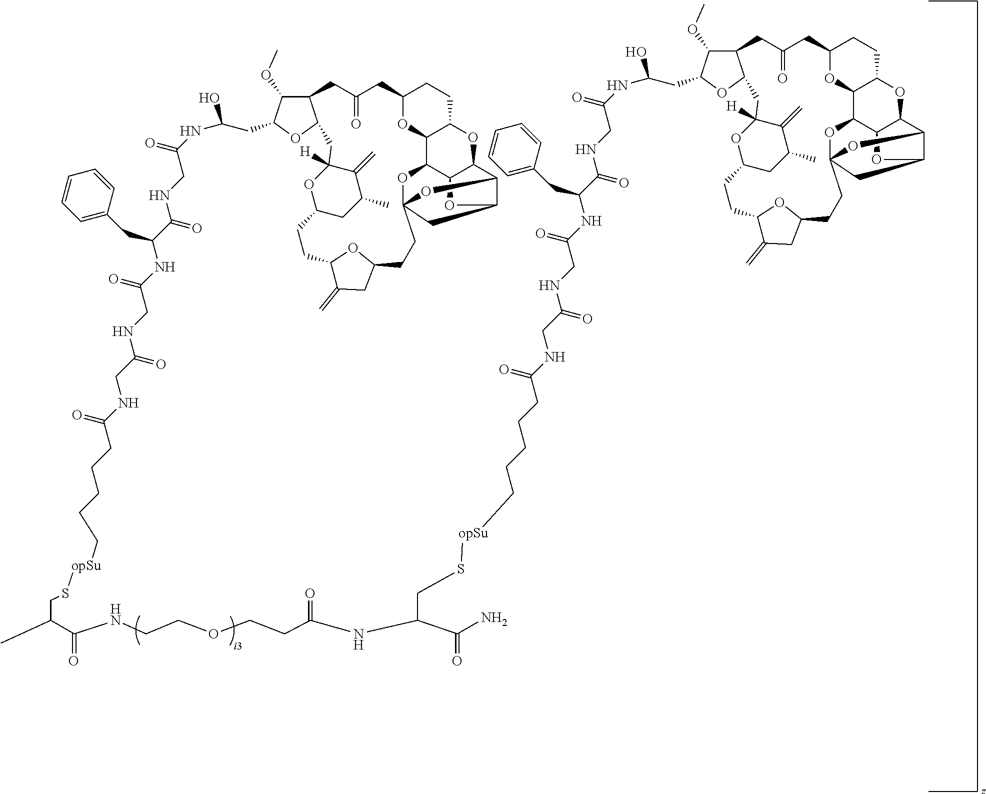

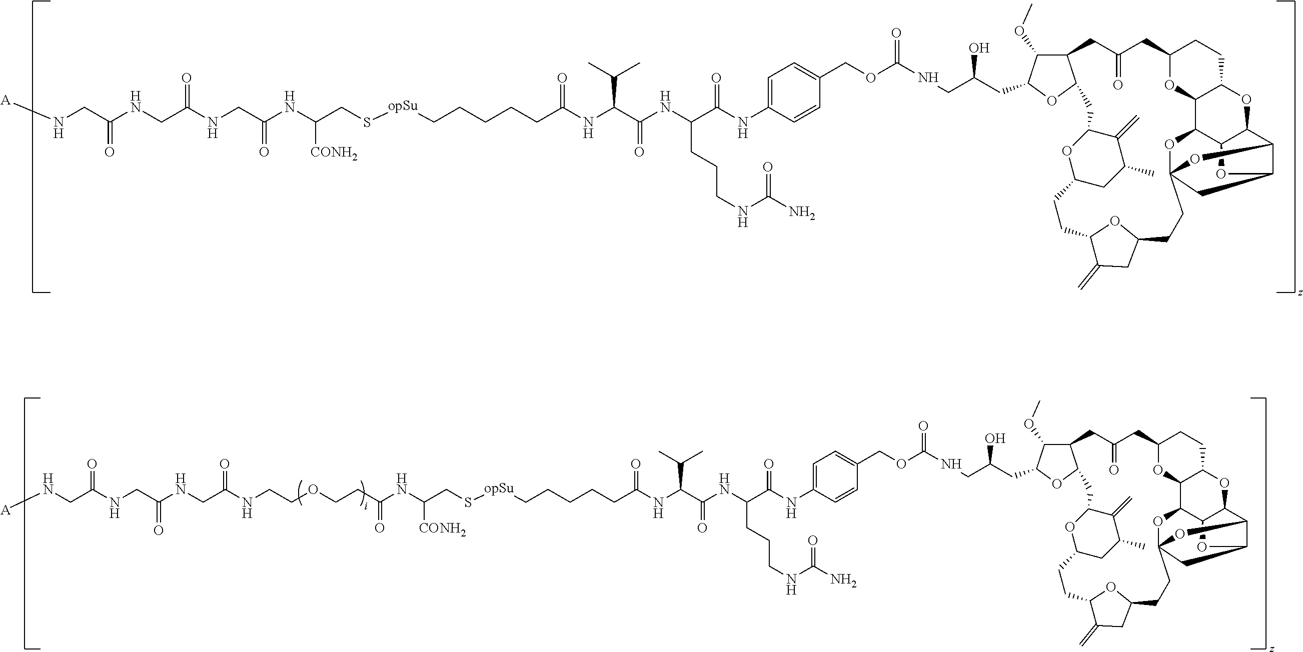

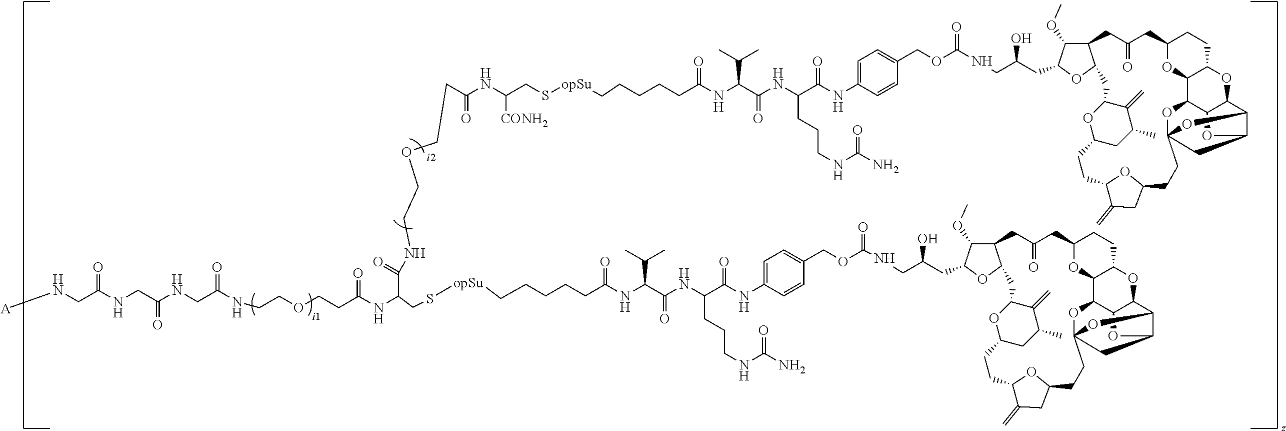

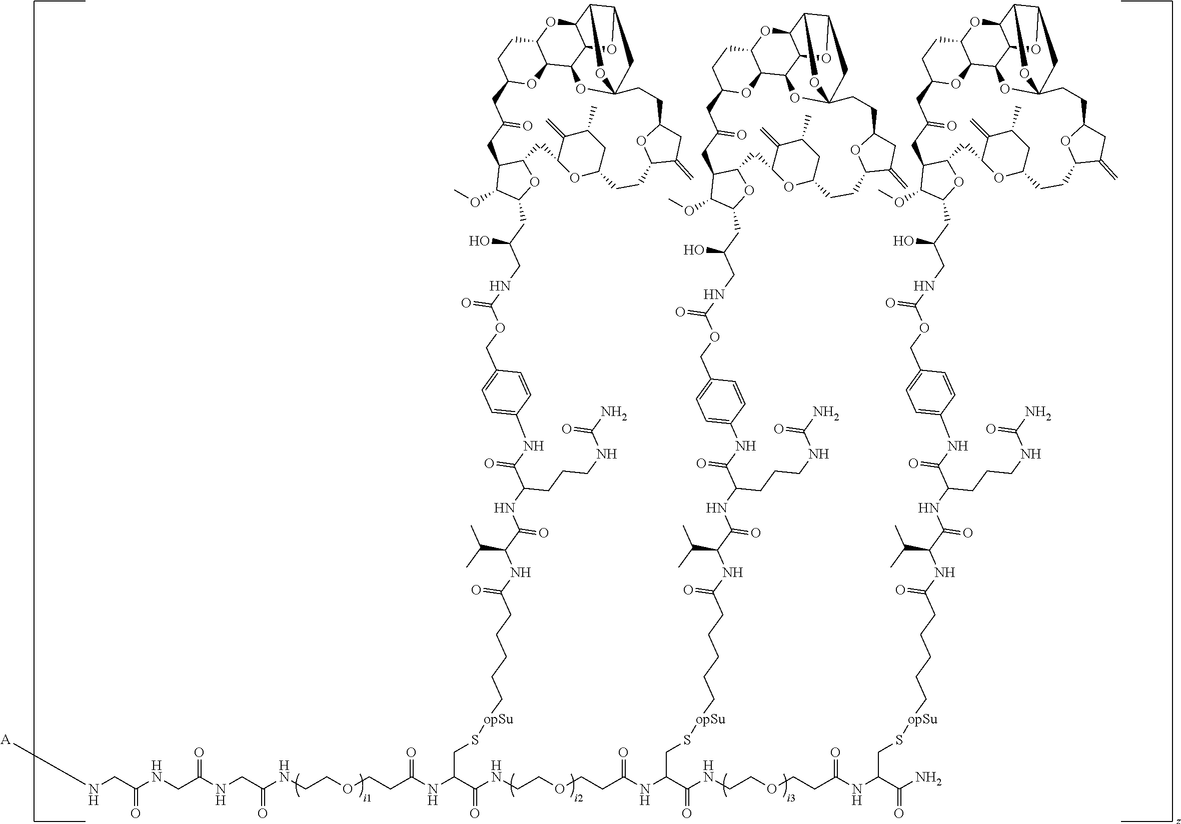

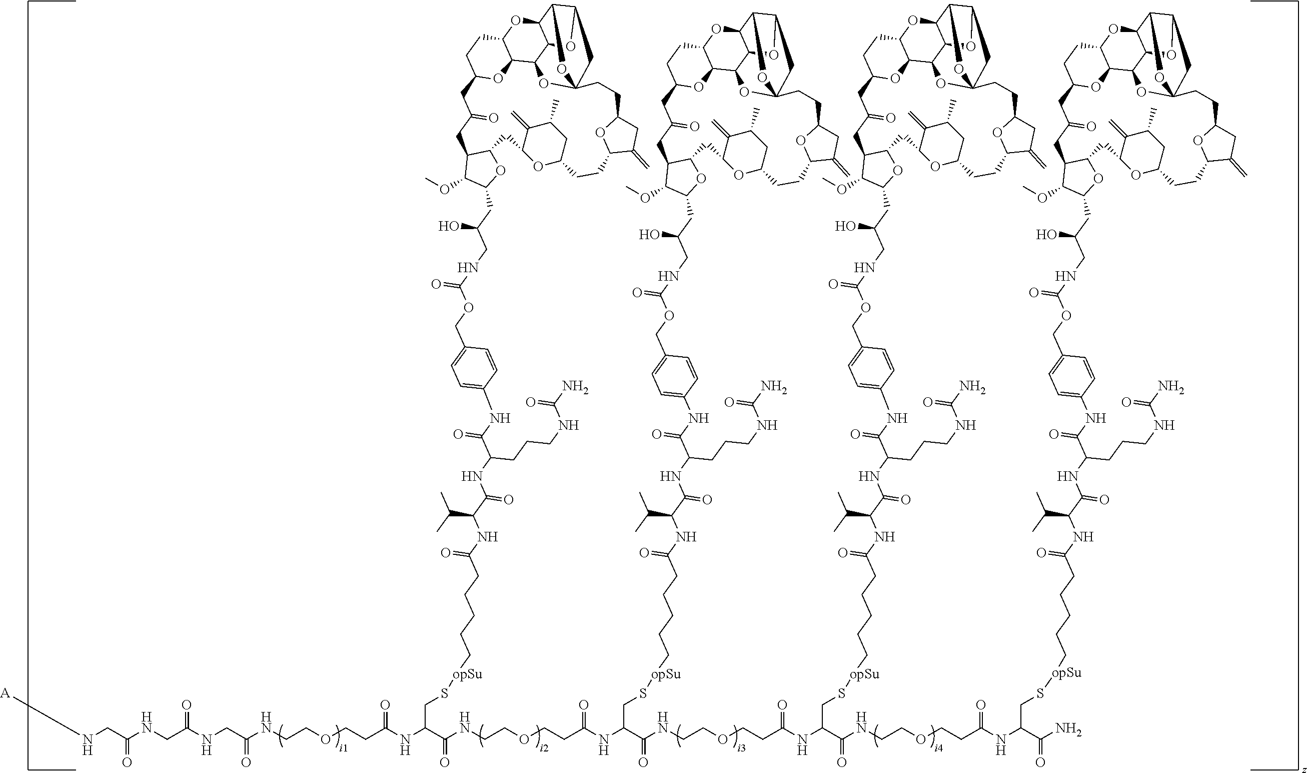

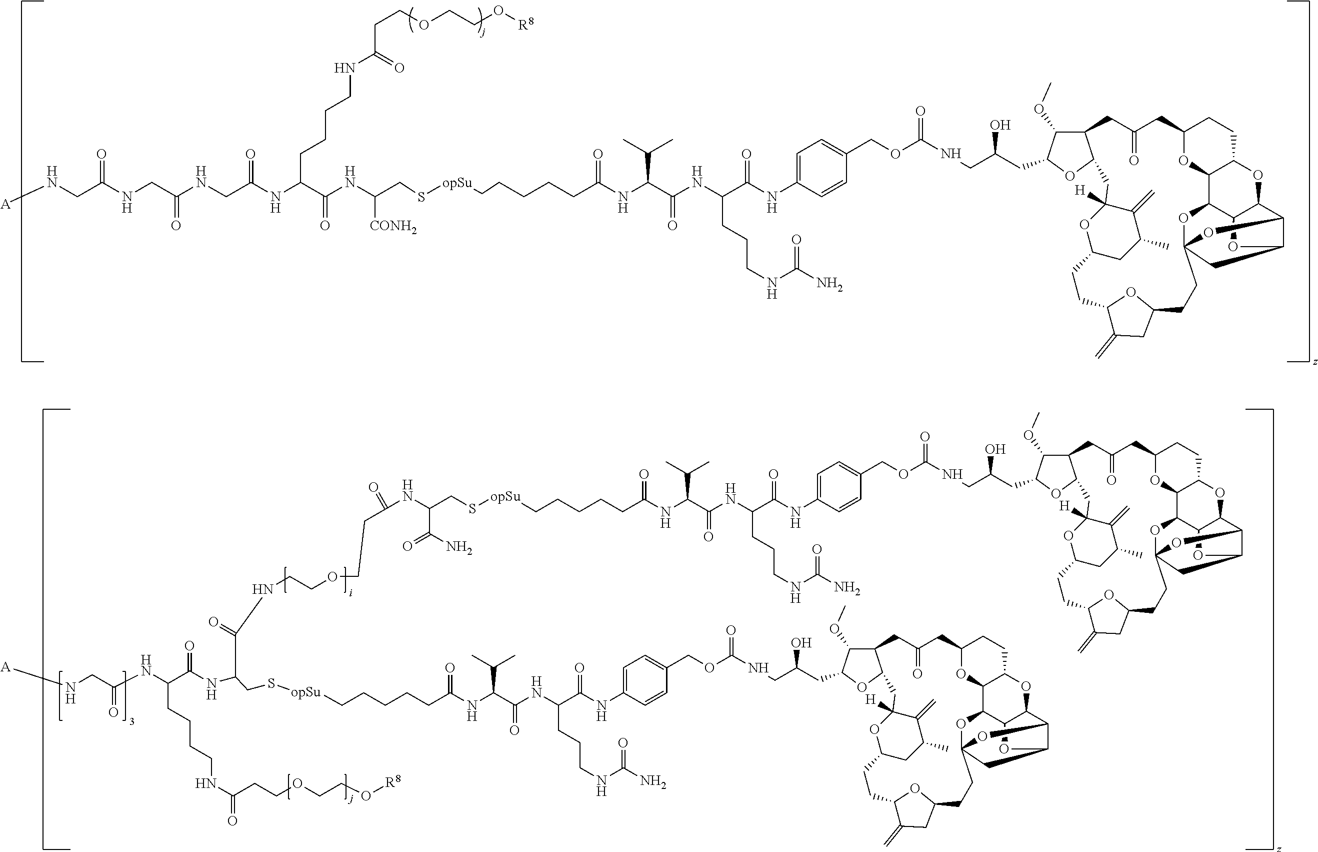

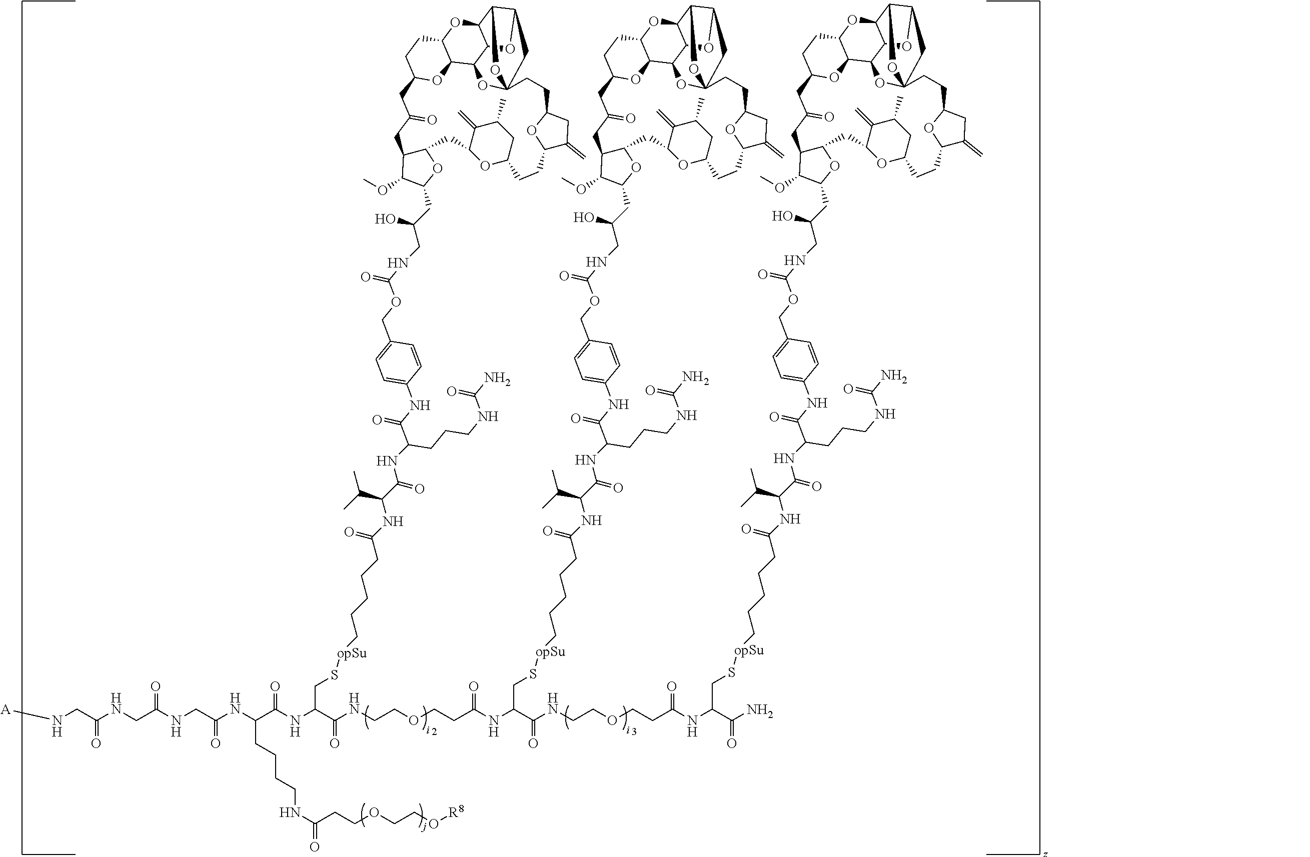

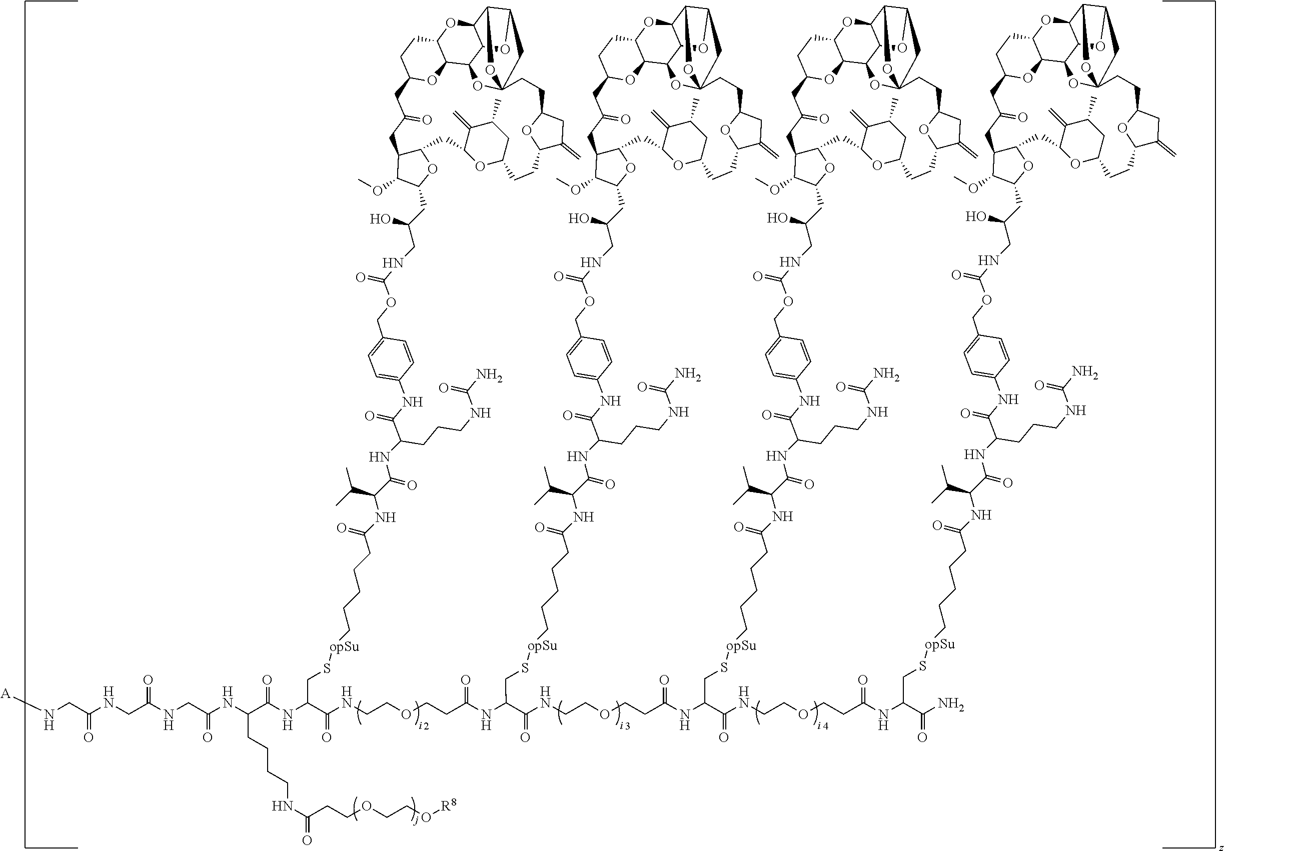

18. A pharmaceutical composition comprising a prophylactically or therapeutically effective amount of a conjugate of claim 10, and at least one pharmaceutically acceptable carrier.

19. A method for treating a disease, comprising administering the pharmaceutical composition of claim 18 to a subject in need thereof; wherein the disease is a tumor or an autoimmune disease;
preferably the disease is a HER2-positive or FGFR3-positive tumor;
preferably, the HER2-positive tumor is selected from breast cancer, gastric cancer, lung cancer, ovarian cancer, and urothelial cancer;
preferably, the FGFR3-positive tumor is selected from multiple myeloma, bladder cancer, urothelial cancer, and glioblastoma.


SEQUENCE LISTING (30 sequences)

SEQ ID NO: 1 - protein, 5 aa, synthetic construct
  REGION 1..5: note = Ab0036 HCDR1
DYTMD

SEQ ID NO: 2 - protein, 17 aa, synthetic construct
  REGION 1..17: note = Ab0036 HCDR2
DVNPNSGGSIYNQRFKG

SEQ ID NO: 3 - protein, 10 aa, synthetic construct
  REGION 1..10: note = Ab0036 HCDR3
NLGPSFYFDY

SEQ ID NO: 4 - protein, 11 aa, synthetic construct
  REGION 1..11: note = Ab0036 LCDR1
KASQDVSIGVA

SEQ ID NO: 5 - protein, 7 aa, synthetic construct
  REGION 1..7: note = Ab0036 LCDR2
SASYRYT

SEQ ID NO: 6 - protein, 9 aa, synthetic construct
  REGION 1..9: note = Ab0036 LCDR3
QQYYIYPYT

SEQ ID NO: 7 - protein, 110 aa, synthetic construct
  REGION 1..110: note = Ab0036 Light chain variable region
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTV

SEQ ID NO: 8 - protein, 119 aa, synthetic construct [sequence omitted]

SEQ ID NO: 9 - protein, 222 aa, synthetic construct
  REGION 1..222: note = Ab0036 Light chain full length
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 10 - protein, 449 aa, synthetic construct
  REGION 1..449: note = Ab0036 Heavy chain full length
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 5 aa, synthetic construct
  REGION 1..5: note = Ab0206 HCDR1
SYGIS

SEQ ID NO: 12 - protein, 17 aa, synthetic construct
  REGION 1..17: note = Ab0206 HCDR2
WVSTYNGDTNYAQKFQG

SEQ ID NO: 13 - protein, 17 aa, synthetic construct
  REGION 1..17: note = Ab0206 HCDR3
VLGYYDSIDGYYYGMDV

SEQ ID NO: 14 - protein, 11 aa, synthetic construct
  REGION 1..11: note = Ab0206 LCDR1
GGNNIGDKSVH

SEQ ID NO: 15 - protein, 7 aa, synthetic construct
  REGION 1..7: note = Ab0206 LCDR2
LDTERPS

SEQ ID NO: 16 - protein, 11 aa, synthetic construct
  REGION 1..11: note = Ab0206 LCDR3
QVWDSGSDHVV

SEQ ID NO: 17 - protein, 111 aa, synthetic construct
  REGION 1..111: note = Ab0206 Light chain variable region
QSVLTQPPSLSVAPGKTATFTCGGNNIGDKSVHWYRQKPGQAPVLVMYLDTERPSGIPERMSGSNFGNTATLTITRVEAEDEADYYCQVWDSGSDHVVFGGGTKLTVLGQP

SEQ ID NO: 18 - protein, 126 aa, synthetic construct
  REGION 1..126: note = Ab0206 Heavy chain variable region
EVQLVQSGAEVKKPGASVKVSCKASGYMFTSYGISWVRQAPGQGLEWMGWVSTYNGDTNYAQKFQGRVTVTTDTSTSTAYMELRSLRSEDTAVYYCARVLGYYDSIDGYYYGMDVWGQGTTVTVSS

SEQ ID NO: 19 - protein, 222 aa, synthetic construct
  REGION 1..222: note = Ab0206 Light chain full length
QSVLTQPPSLSVAPGKTATFTCGGNNIGDKSVHWYRQKPGQAPVLVMYLDTERPSGIPERMSGSNFGNTATLTITRVEAEDEADYYCQVWDSGSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECSGALPETGG

SEQ ID NO: 20 - protein, 456 aa, synthetic construct
  REGION 1..456: note = Ab0206 Heavy chain full length
EVQLVQSGAEVKKPGASVKVSCKASGYMFTSYGISWVRQAPGQGLEWMGWVSTYNGDTNYAQKFQGRVTVTTDTSTSTAYMELRSLRSEDTAVYYCARVLGYYDSIDGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 5 aa, synthetic construct
  REGION 1..5: note = Ab0001 HCDR1
DTYIH

SEQ ID NO: 22 - protein, 17 aa, synthetic construct
  REGION 1..17: note = Ab0001 HCDR2
RIYPTNGYTRYADSVKG

SEQ ID NO: 23 - protein, 11 aa, synthetic construct
  REGION 1..11: note = Ab0001 HCDR3
WGGDGFYAMDY

SEQ ID NO: 24 - protein, 11 aa, synthetic construct
  REGION 1..11: note = Ab0001 LCDR1
RASQDVNTAVA

SEQ ID NO: 25 - protein, 7 aa, synthetic construct
  REGION 1..7: note = Ab0001 LCDR2
SASFLYS

SEQ ID NO: 26 - protein, 9 aa, synthetic construct
  REGION 1..9: note = Ab0001 LCDR3
QQHYTTPPT

SEQ ID NO: 27 - protein, 110 aa, synthetic construct
  REGION 1..110: note = Ab0001 Light chain variable region
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTV

SEQ ID NO: 28 - protein, 120 aa, synthetic construct [sequence omitted]

SEQ ID NO: 29 - protein, 222 aa, synthetic construct
  REGION 1..222: note = Ab0001 Light chain full length
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 30 - protein, 450 aa, synthetic construct
  REGION 1..450: note = Ab0001 Heavy chain full length
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK